SEQUENCE LISTING (260 sequences)

SEQ ID NO: 1 - protein, 35 aa, Stichodactyla helianthus
RSCIDTIPKSRCTAFQCKHSMKYRLSFCRKTCGTC

SEQ ID NO: 2 - protein, 35 aa, Artificial Sequence
  ShK-Based Peptide
RSCIDTIPKSRCTAFQSKHSMKYRLSFCRKTSGTC

SEQ ID NO: 3 - protein, 35 aa, Artificial Sequence
  ShK-Based Peptide
RSSIDTIPKSRCTAFQCKHSMKYRLSFCRKTCGTS

SEQ ID NO: 4 - protein, 35 aa, Artificial Sequence
  ShK-Based Peptide
SSCIDTIPKSRCTAFQCKHSMKYRLSFCRKTCGTC

SEQ ID NO: 5 - protein, 35 aa, Artificial Sequence
  ShK-Based Peptide
XSCIDTIPKSRCTAFQCKHSMKYRLSFCRKTCGTC

SEQ ID NO: 6 - protein, 34 aa, Artificial Sequence
  ShK-Based Peptide
SCIDTIPKSRCTAFQCKHSMKYRLSFCRKTCGTC

SEQ ID NO: 7 - protein, 33 aa, Artificial Sequence
  ShK-Based Peptide
CIDTIPKSRCTAFQCKHSMKYRLSFCRKTCGTC

SEQ ID NO: 8 - protein, 35 aa, Artificial Sequence
  ShK-Based Peptide
ASCIDTIPKSRCTAFQCKHSMKYRLSFCRKTCGTC

SEQ ID NO: 9 - protein, 34 aa, Artificial Sequence
  ShK-Based Peptide
QCIDTIPKSRCTAFQCKHSMKYRLSFCRKTCGTC

SEQ ID NO: 10 - protein, 34 aa, Artificial Sequence
  ShK-Based Peptide
ACIDTIPKSRCTAFQCKHSMKYRLSFCRKTCGTC

SEQ ID NO: 11 - protein, 34 aa, Artificial Sequence
  ShK-Based Peptide
TCIDTIPKSRCTAFQCKHSMKYRLSFCRKTCGTC

SEQ ID NO: 12 - protein, 35 aa, Artificial Sequence
  ShK-Based Peptide
RQCIDTIPKSRCTAFQCKHSMKYRLSFCRKTCGTC

SEQ ID NO: 13 - protein, 35 aa, Artificial Sequence
  ShK-Based Peptide
RACIDTIPKSRCTAFQCKHSMKYRLSFCRKTCGTC

SEQ ID NO: 14 - protein, 35 aa, Artificial Sequence
  ShK-Based Peptide
RTCIDTIPKSRCTAFQCKHSMKYRLSFCRKTCGTC

SEQ ID NO: 15 - protein, 35 aa, Artificial Sequence
  ShK-Based Peptide
AQCIDTIPKSRCTAFQCKHSMKYRLSFCRKTCGTC

SEQ ID NO: 16 - protein, 35 aa, Artificial Sequence
  ShK-Based Peptide
AACIDTIPKSRCTAFQCKHSMKYRLSFCRKTCGTC

SEQ ID NO: 17 - protein, 35 aa, Artificial Sequence
  ShK-Based Peptide
ATCIDTIPKSRCTAFQCKHSMKYRLSFCRKTCGTC

SEQ ID NO: 18 - protein, 35 aa, Artificial Sequence
  ShK-Based Peptide
RSCADTIPKSRCTAFQCKHSMKYRLSFCRKTCGTC

SEQ ID NO: 19 - protein, 35 aa, Artificial Sequence
  ShK-Based Peptide
RSCADTIPKSRCTAAQCKHSMKYRLSFCRKTCGTC

SEQ ID NO: 20 - protein, 35 aa, Artificial Sequence
  ShK-Based Peptide
RSCADTIPKSRCTAAQCKHSMKYRASFCRKTCGTC

SEQ ID NO: 21 - protein, 35 aa, Artificial Sequence
  ShK-Based Peptide
RSCIDAIPKSRCTAFQCKHSMKYRLSFCRKTCGTC

SEQ ID NO: 22 - protein, 35 aa, Artificial Sequence; identical to SEQ ID NO: 1
  ShK-Based Peptide

SEQ ID NO: 23 - protein, 35 aa, Artificial Sequence
  ShK-Based Peptide
RSCIDYIPKSRCTAFQCKHSMKYRLSFCRKTCGTC

SEQ ID NO: 24 - protein, 35 aa, Artificial Sequence
  ShK-Based Peptide
RSCIDLIPKSRCTAFQCKHSMKYRLSFCRKTCGTC

SEQ ID NO: 25 - protein, 35 aa, Artificial Sequence
  ShK-Based Peptide
RSCIDTAPKSRCTAFQCKHSMKYRLSFCRKTCGTC

SEQ ID NO: 26 - protein, 35 aa, Artificial Sequence; identical to SEQ ID NO: 18
  ShK-Based Peptide

SEQ ID NO: 27 - protein, 35 aa, Artificial Sequence
  ShK-Based Peptide
RSCIDTIAKSRCTAFQCKHSMKYRLSFCRKTCGTC

SEQ ID NO: 28 - protein, 35 aa, Artificial Sequence
  ShK-Based Peptide
RSCIDTIPASRCTAFQCKHSMKYRLSFCRKTCGTC

SEQ ID NO: 29 - protein, 35 aa, Artificial Sequence
  ShK-Based Peptide
RSCIDTIPESRCTAFQCKHSMKYRLSFCRKTCGTC

SEQ ID NO: 30 - protein, 35 aa, Artificial Sequence
  ShK-Based Peptide
RSCIDTIPQSRCTAFQCKHSMKYRLSFCRKTCGTC

SEQ ID NO: 31 - protein, 35 aa, Artificial Sequence
  ShK-Based Peptide
RSCIDTIPKARCTAFQCKHSMKYRLSFCRKTCGTC

SEQ ID NO: 32 - protein, 35 aa, Artificial Sequence
  ShK-Based Peptide
RSCIDTIPKSACTAFQCKHSMKYRLSFCRKTCGTC

SEQ ID NO: 33 - protein, 35 aa, Artificial Sequence
  ShK-Based Peptide
RSCIDTIPKSECTAFQCKHSMKYRLSFCRKTCGTC

SEQ ID NO: 34 - protein, 35 aa, Artificial Sequence
  ShK-Based Peptide
RSCIDTIPKSQCTAFQCKHSMKYRLSFCRKTCGTC

SEQ ID NO: 35 - protein, 35 aa, Artificial Sequence
  ShK-Based Peptide
RSCIDTIPKSRCAAFQCKHSMKYRLSFCRKTCGTC

SEQ ID NO: 36 - protein, 35 aa, Artificial Sequence
  ShK-Based Peptide
RSCIDTIPKSRCTAAQCKHSMKYRLSFCRKTCGTC

SEQ ID NO: 37 - protein, 35 aa, Artificial Sequence
  ShK-Based Peptide
RSCIDTIPKSRCTAWQCKHSMKYRLSFCRKTCGTC

SEQ ID NO: 38 - protein, 35 aa, Artificial Sequence
  ShK-Based Peptide
RSCIDTIPKSRCTAXQCKHSMKYRLSFCRKTCGTC

SEQ ID NO: 39 - protein, 35 aa, Artificial Sequence
  ShK-Based Peptide
RSCIDTIPKSRCTAAQCKHSMKYRASFCRKTCGTC

SEQ ID NO: 40 - protein, 35 aa, Artificial Sequence
  ShK-Based Peptide
RSCIDTIPKSRCTAFACKHSMKYRLSFCRKTCGTC

SEQ ID NO: 41 - protein, 35 aa, Artificial Sequence
  ShK-Based Peptide
RSCIDTIPKSRCTAFECKHSMKYRLSFCRKTCGTC

SEQ ID NO: 42 - protein, 35 aa, Artificial Sequence
  ShK-Based Peptide
RSCIDTIPKSRCTAFQCAHSMKYRLSFCRKTCGTC

SEQ ID NO: 43 - protein, 35 aa, Artificial Sequence
  ShK-Based Peptide
RSCIDTIPKSRCTAFQCEHSMKYRLSFCRKTCGTC

SEQ ID NO: 44 - protein, 35 aa, Artificial Sequence
  ShK-Based Peptide
RSCIDTIPKSRCTAFQCKASMKYRLSFCRKTCGTC

SEQ ID NO: 45 - protein, 35 aa, Artificial Sequence
  ShK-Based Peptide
RSCIDTIPKSRCTAFQCKKSMKYRLSFCRKTCGTC

SEQ ID NO: 46 - protein, 35 aa, Artificial Sequence
  ShK-Based Peptide
RSCIDTIPKSRCTAFQCKHAMKYRLSFCRKTCGTC

SEQ ID NO: 47 - protein, 35 aa, Artificial Sequence
  ShK-Based Peptide
RSCIDTIPKSRCTAFQCKHSAKYRLSFCRKTCGTC

SEQ ID NO: 48 - protein, 35 aa, Artificial Sequence
  ShK-Based Peptide
RSCIDTIPKSRCTAFQCKHSXKYRLSFCRKTCGTC

SEQ ID NO: 49 - protein, 35 aa, Artificial Sequence; identical to SEQ ID NO: 48
  ShK-Based Peptide

SEQ ID NO: 50 - protein, 35 aa, Artificial Sequence
  ShK-Based Peptide
RSCIDTIPKSRCTAFQCKHSMAYRLSFCRKTCGTC

SEQ ID NO: 51 - protein, 35 aa, Artificial Sequence
  ShK-Based Peptide
RSCIDTIPKSRCTAFQCKHSMEYRLSFCRKTCGTC

SEQ ID NO: 52 - protein, 35 aa, Artificial Sequence
  ShK-Based Peptide
RSCIDTIPKSRCTAFQCKHSMRYRLSFCRKTCGTC

SEQ ID NO: 53 - protein, 35 aa, Artificial Sequence
  ShK-Based Peptide
RSCIDTIPKSRCTAFQCKHSMXYRLSFCRKTCGTC

SEQ ID NO: 54 - protein, 35 aa, Artificial Sequence; identical to SEQ ID NO: 53
  ShK-Based Peptide

SEQ ID NO: 55 - protein, 35 aa, Artificial Sequence; identical to SEQ ID NO: 53
  ShK-Based Peptide

SEQ ID NO: 56 - protein, 35 aa, Artificial Sequence; identical to SEQ ID NO: 53
  ShK-Based Peptide

SEQ ID NO: 57 - protein, 35 aa, Artificial Sequence; identical to SEQ ID NO: 53
  ShK-Based Peptide

SEQ ID NO: 58 - protein, 35 aa, Artificial Sequence
  ShK-Based Peptide
RSCIDTIPKSRCTAFQCKHSMKARLSFCRKTCGTC

SEQ ID NO: 59 - protein, 35 aa, Artificial Sequence
  ShK-Based Peptide
RSCIDTIPKSRCTAFQCKHSMKSRLSFCRKTCGTC

SEQ ID NO: 60 - protein, 35 aa, Artificial Sequence
  ShK-Based Peptide
RSCIDTIPKSRCTAFQCKHSMKFRLSFCRKTCGTC

SEQ ID NO: 61 - protein, 35 aa, Artificial Sequence
  ShK-Based Peptide
RSCIDTIPKSRCTAFQCKHSMKXRLSFCRKTCGTC

SEQ ID NO: 62 - protein, 35 aa, Artificial Sequence; identical to SEQ ID NO: 61
  ShK-Based Peptide

SEQ ID NO: 63 - protein, 36 aa, Artificial Sequence
  ShK-Based Peptide
RSCIDTIPKSRCTAFQCKHSMKXRLSFCRKTCGTCC

SEQ ID NO: 64 - protein, 35 aa, Artificial Sequence; identical to SEQ ID NO: 61
  ShK-Based Peptide

SEQ ID NO: 65 - protein, 35 aa, Artificial Sequence
  ShK-Based Peptide
RSCIDTIPKSRCTAFQCKHSMKYALSFCRKTCGTC

SEQ ID NO: 66 - protein, 35 aa, Artificial Sequence
  ShK-Based Peptide
RSCIDTIPKSRCTAFQCKHSMKYELSFCRKTCGTC

SEQ ID NO: 67 - protein, 35 aa, Artificial Sequence
  ShK-Based Peptide
RSCIDTIPKSRCTAFQCKHSMKYRASFCRKTCGTC

SEQ ID NO: 68 - protein, 35 aa, Artificial Sequence
  ShK-Based Peptide
RSCIDTIPKSRCTAFQCKHSMKYRLAFCRKTCGTC

SEQ ID NO: 69 - protein, 35 aa, Artificial Sequence
  ShK-Based Peptide
RSCIDTIPKSRCTAFQCKHSMKYRLSACRKTCGTC

SEQ ID NO: 70 - protein, 35 aa, Artificial Sequence
  ShK-Based Peptide
RSCIDTIPKSRCTAFQCKHSMKYRLSXCRKTCGTC

SEQ ID NO: 71 - protein, 35 aa, Artificial Sequence
  ShK-Based Peptide
RSCIDTIPKSRCTAFQCKHSMKYRLSFCAKTCGTC

SEQ ID NO: 72 - protein, 35 aa, Artificial Sequence
  ShK-Based Peptide
RSCIDTIPKSRCTAFQCKHSMKYRLSFCRATCGTC

SEQ ID NO: 73 - protein, 35 aa, Artificial Sequence
  ShK-Based Peptide
RSCIDTIPKSRCTAFQCKHSMKYRLSFCRKACGTC

SEQ ID NO: 74 - protein, 35 aa, Artificial Sequence
  ShK-Based Peptide
RSCIDTIPKSRCTAFQCKHSMKYRLSFCRKTCGAC

SEQ ID NO: 75 - protein, 34 aa, Artificial Sequence
  ShK-Based Peptide
SCADTIPKSRCTAFQCKHSMKYRLSFCRKTCGTC

SEQ ID NO: 76 - protein, 34 aa, Artificial Sequence
  ShK-Based Peptide
SCADTIPKSRCTAAQCKHSMKYRLSFCRKTCGTC

SEQ ID NO: 77 - protein, 34 aa, Artificial Sequence
  ShK-Based Peptide
SCADTIPKSRCTAAQCKHSMKYRASFCRKTCGTC

SEQ ID NO: 78 - protein, 34 aa, Artificial Sequence
  ShK-Based Peptide
SCIDAIPKSRCTAFQCKHSMKYRLSFCRKTCGTC

SEQ ID NO: 79 - protein, 34 aa, Artificial Sequence
  ShK-Based Peptide
SCIDTAPKSRCTAFQCKHSMKYRLSFCRKTCGTC

SEQ ID NO: 80 - protein, 34 aa, Artificial Sequence
  ShK-Based Peptide
SCIDTIAKSRCTAFQCKHSMKYRLSFCRKTCGTC

SEQ ID NO: 81 - protein, 34 aa, Artificial Sequence
  ShK-Based Peptide
SCIDTIPASRCTAFQCKHSMKYRLSFCRKTCGTC

SEQ ID NO: 82 - protein, 34 aa, Artificial Sequence
  ShK-Based Peptide
SCIDTIPESRCTAFQCKHSMKYRLSFCRKTCGTC

SEQ ID NO: 83 - protein, 34 aa, Artificial Sequence
  ShK-Based Peptide
SCIDTIPQSRCTAFQCKHSMKYRLSFCRKTCGTC

SEQ ID NO: 84 - protein, 34 aa, Artificial Sequence
  ShK-Based Peptide
SCIDTIPKARCTAFQCKHSMKYRLSFCRKTCGTC

SEQ ID NO: 85 - protein, 34 aa, Artificial Sequence
  ShK-Based Peptide
SCIDTIPKSACTAFQCKHSMKYRLSFCRKTCGTC

SEQ ID NO: 86 - protein, 34 aa, Artificial Sequence
  ShK-Based Peptide
SCIDTIPKSECTAFQCKHSMKYRLSFCRKTCGTC

SEQ ID NO: 87 - protein, 34 aa, Artificial Sequence
  ShK-Based Peptide
SCIDTIPKSQCTAFQCKHSMKYRLSFCRKTCGTC

SEQ ID NO: 88 - protein, 34 aa, Artificial Sequence
  ShK-Based Peptide
SCIDTIPKSRCAAFQCKHSMKYRLSFCRKTCGTC

SEQ ID NO: 89 - protein, 34 aa, Artificial Sequence
  ShK-Based Peptide
SCIDTIPKSRCTAAQCKHSMKYRLSFCRKTCGTC

SEQ ID NO: 90 - protein, 34 aa, Artificial Sequence
  ShK-Based Peptide
SCIDTIPKSRCTAWQCKHSMKYRLSFCRKTCGTC

SEQ ID NO: 91 - protein, 34 aa, Artificial Sequence
  ShK-Based Peptide
SCIDTIPKSRCTAXQCKHSMKYRLSFCRKTCGTC

SEQ ID NO: 92 - protein, 34 aa, Artificial Sequence
  ShK-Based Peptide
SCIDTIPKSRCTAAQCKHSMKYRASFCRKTCGTC

SEQ ID NO: 93 - protein, 34 aa, Artificial Sequence
  ShK-Based Peptide
SCIDTIPKSRCTAFACKHSMKYRLSFCRKTCGTC

SEQ ID NO: 94 - protein, 34 aa, Artificial Sequence
  ShK-Based Peptide
SCIDTIPKSRCTAFECKHSMKYRLSFCRKTCGTC

SEQ ID NO: 95 - protein, 34 aa, Artificial Sequence
  ShK-Based Peptide
SCIDTIPKSRCTAFQCAHSMKYRLSFCRKTCGTC

SEQ ID NO: 96 - protein, 34 aa, Artificial Sequence
  ShK-Based Peptide
SCIDTIPKSRCTAFQCEHSMKYRLSFCRKTCGTC

SEQ ID NO: 97 - protein, 34 aa, Artificial Sequence
  ShK-Based Peptide
SCIDTIPKSRCTAFQCKASMKYRLSFCRKTCGTC

SEQ ID NO: 98 - protein, 34 aa, Artificial Sequence
  ShK-Based Peptide
SCIDTIPKSRCTAFQCKKSMKYRLSFCRKTCGTC

SEQ ID NO: 99 - protein, 34 aa, Artificial Sequence
  ShK-Based Peptide
SCIDTIPKSRCTAFQCKHAMKYRLSFCRKTCGTC

SEQ ID NO: 100 - protein, 34 aa, Artificial Sequence
  ShK-Based Peptide
SCIDTIPKSRCTAFQCKHSAKYRLSFCRKTCGTC

SEQ ID NO: 101 - protein, 34 aa, Artificial Sequence
  ShK-Based Peptide
SCIDTIPKSRCTAFQCKHSXKYRLSFCRKTCGTC

SEQ ID NO: 102 - protein, 34 aa, Artificial Sequence; identical to SEQ ID NO: 101
  ShK-Based Peptide

SEQ ID NO: 103 - protein, 34 aa, Artificial Sequence
  ShK-Based Peptide
SCIDTIPKSRCTAFQCKHSMAYRLSFCRKTCGTC

SEQ ID NO: 104 - protein, 34 aa, Artificial Sequence
  ShK-Based Peptide
SCIDTIPKSRCTAFQCKHSMEYRLSFCRKTCGTC

SEQ ID NO: 105 - protein, 34 aa, Artificial Sequence
  ShK-Based Peptide
SCIDTIPKSRCTAFQCKHSMRYRLSFCRKTCGTC

SEQ ID NO: 106 - protein, 34 aa, Artificial Sequence
  ShK-Based Peptide
SCIDTIPKSRCTAFQCKHSMXYRLSFCRKTCGTC

SEQ ID NO: 107 - protein, 34 aa, Artificial Sequence; identical to SEQ ID NO: 106
  ShK-Based Peptide

SEQ ID NO: 108 - protein, 34 aa, Artificial Sequence; identical to SEQ ID NO: 106
  ShK-Based Peptide

SEQ ID NO: 109 - protein, 34 aa, Artificial Sequence; identical to SEQ ID NO: 106
  ShK-Based Peptide

SEQ ID NO: 110 - protein, 34 aa, Artificial Sequence; identical to SEQ ID NO: 106
  ShK-Based Peptide

SEQ ID NO: 111 - protein, 34 aa, Artificial Sequence
  ShK-Based Peptide
SCIDTIPKSRCTAFQCKHSMKARLSFCRKTCGTC

SEQ ID NO: 112 - protein, 34 aa, Artificial Sequence
  ShK-Based Peptide
SCIDTIPKSRCTAFQCKHSMKSRLSFCRKTCGTC

SEQ ID NO: 113 - protein, 34 aa, Artificial Sequence
  ShK-Based Peptide
SCIDTIPKSRCTAFQCKHSMKFRLSFCRKTCGTC

SEQ ID NO: 114 - protein, 34 aa, Artificial Sequence
  ShK-Based Peptide
SCIDTIPKSRCTAFQCKHSMKXRLSFCRKTCGTC

SEQ ID NO: 115 - protein, 34 aa, Artificial Sequence; identical to SEQ ID NO: 114
  ShK-Based Peptide

SEQ ID NO: 116 - protein, 34 aa, Artificial Sequence; identical to SEQ ID NO: 114
  ShK-Based Peptide

SEQ ID NO: 117 - protein, 33 aa, Artificial Sequence
  ShK-Based Peptide
SCIDTIPKSRCTAFQCKHSMKXRLSFCRKTCGT

SEQ ID NO: 118 - protein, 34 aa, Artificial Sequence
  ShK-Based Peptide
SCIDTIPKSRCTAFQCKHSMKYALSFCRKTCGTC

SEQ ID NO: 119 - protein, 34 aa, Artificial Sequence
  ShK-Based Peptide
SCIDTIPKSRCTAFQCKHSMKYELSFCRKTCGTC

SEQ ID NO: 120 - protein, 34 aa, Artificial Sequence
  ShK-Based Peptide
SCIDTIPKSRCTAFQCKHSMKYRASFCRKTCGTC

SEQ ID NO: 121 - protein, 34 aa, Artificial Sequence
  ShK-Based Peptide
SCIDTIPKSRCTAFQCKHSMKYRLAFCRKTCGTC

SEQ ID NO: 122 - protein, 34 aa, Artificial Sequence
  ShK-Based Peptide
SCIDTIPKSRCTAFQCKHSMKYRLSACRKTCGTC

SEQ ID NO: 123 - protein, 34 aa, Artificial Sequence
  ShK-Based Peptide
SCIDTIPKSRCTAFQCKHSMKYRLSXCRKTCGTC

SEQ ID NO: 124 - protein, 34 aa, Artificial Sequence
  ShK-Based Peptide
SCIDTIPKSRCTAFQCKHSMKYRLSFCAKTCGTC

SEQ ID NO: 125 - protein, 34 aa, Artificial Sequence
  ShK-Based Peptide
SCIDTIPKSRCTAFQCKHSMKYRLSFCRATCGTC

SEQ ID NO: 126 - protein, 34 aa, Artificial Sequence
  ShK-Based Peptide
SCIDTIPKSRCTAFQCKHSMKYRLSFCRKACGTC

SEQ ID NO: 127 - protein, 34 aa, Artificial Sequence
  ShK-Based Peptide
SCIDTIPKSRCTAFQCKHSMKYRLSFCRKTCGAC

SEQ ID NO: 128 - protein, 35 aa, Artificial Sequence
  ShK-Based Peptide
YSCIDTIPKSRCTAFQCKHSMKYRLSFCRKTCGTC

SEQ ID NO: 129 - protein, 35 aa, Artificial Sequence
  ShK-Based Peptide
KSCIDTIPKSRCTAFQCKHSMKYRLSFCRKTCGTC

SEQ ID NO: 130 - protein, 35 aa, Artificial Sequence
  ShK-Based Peptide
HSCIDTIPKSRCTAFQCKHSMKYRLSFCRKTCGTC

SEQ ID NO: 131 - protein, 35 aa, Artificial Sequence
  ShK-Based Peptide
QSCIDTIPKSRCTAFQCKHSMKYRLSFCRKTCGTC

SEQ ID NO: 132 - protein, 37 aa, Artificial Sequence
  ShK-Based Peptide
PPRSCIDTIPKSRCTAFQCKHSMKYRLSFCRKTCGTC

SEQ ID NO: 133 - protein, 36 aa, Artificial Sequence
  ShK-Based Peptide
MRSCIDTIPKSRCTAFQCKHSMKYRLSFCRKTCGTC

SEQ ID NO: 134 - protein, 36 aa, Artificial Sequence
  ShK-Based Peptide
GRSCIDTIPKSRCTAFQCKHSMKYRLSFCRKTCGTC

SEQ ID NO: 135 - protein, 35 aa, Artificial Sequence
  ShK-Based Peptide
YSCIDTIPKSRCTAFQCKHSMAYRLSFCRKTCGTC

SEQ ID NO: 136 - protein, 35 aa, Artificial Sequence
  ShK-Based Peptide
KSCIDTIPKSRCTAFQCKHSMAYRLSFCRKTCGTC

SEQ ID NO: 137 - protein, 35 aa, Artificial Sequence
  ShK-Based Peptide
HSCIDTIPKSRCTAFQCKHSMAYRLSFCRKTCGTC

SEQ ID NO: 138 - protein, 35 aa, Artificial Sequence
  ShK-Based Peptide
QSCIDTIPKSRCTAFQCKHSMAYRLSFCRKTCGTC

SEQ ID NO: 139 - protein, 37 aa, Artificial Sequence
  ShK-Based Peptide
PPRSCIDTIPKSRCTAFQCKHSMAYRLSFCRKTCGTC

SEQ ID NO: 140 - protein, 36 aa, Artificial Sequence
  ShK-Based Peptide
MRSCIDTIPKSRCTAFQCKHSMAYRLSFCRKTCGTC

SEQ ID NO: 141 - protein, 36 aa, Artificial Sequence
  ShK-Based Peptide
GRSCIDTIPKSRCTAFQCKHSMAYRLSFCRKTCGTC

SEQ ID NO: 142 - protein, 35 aa, Artificial Sequence
  ShK-Based Peptide
RSCIDTIPASRCTAFQCKHSMAYRLSFCRKTCGTC

SEQ ID NO: 143 - protein, 34 aa, Artificial Sequence
  ShK-Based Peptide
SCIDTIPASRCTAFQCKHSMAYRLSFCRKTCGTC

SEQ ID NO: 144 - protein, 35 aa, Artificial Sequence
  ShK-Based Peptide
RSCIDTIPVSRCTAFQCKHSMKYRLSFCRKTCGTC

SEQ ID NO: 145 - protein, 35 aa, Artificial Sequence
  ShK-Based Peptide
RSCIDTIPVSRCTAFQCKHSMAYRLSFCRKTCGTC

SEQ ID NO: 146 - protein, 34 aa, Artificial Sequence
  ShK-Based Peptide
SCIDTIPVSRCTAFQCKHSMKYRLSFCRKTCGTC

SEQ ID NO: 147 - protein, 34 aa, Artificial Sequence
  ShK-Based Peptide
SCIDTIPVSRCTAFQCKHSMAYRLSFCRKTCGTC

SEQ ID NO: 148 - protein, 35 aa, Artificial Sequence
  ShK-Based Peptide
RSCIDTIPESRCTAFQCKHSMAYRLSFCRKTCGTC

SEQ ID NO: 149 - protein, 34 aa, Artificial Sequence
  ShK-Based Peptide
SCIDTIPESRCTAFQCKHSMAYRLSFCRKTCGTC

SEQ ID NO: 150 - protein, 35 aa, Artificial Sequence
  ShK-Based Peptide
RSCIDTIPKSACTAFQCKHSMAYRLSFCRKTCGTC

SEQ ID NO: 151 - protein, 34 aa, Artificial Sequence
  ShK-Based Peptide
SCIDTIPKSACTAFQCKHSMAYRLSFCRKTCGTC

SEQ ID NO: 152 - protein, 35 aa, Artificial Sequence
  ShK-Based Peptide
RSCIDTIPKSECTAFQCKHSMAYRLSFCRKTCGTC

SEQ ID NO: 153 - protein, 34 aa, Artificial Sequence
  ShK-Based Peptide
SCIDTIPKSECTAFQCKHSMAYRLSFCRKTCGTC

SEQ ID NO: 154 - protein, 35 aa, Artificial Sequence
  ShK-Based Peptide
RSCIDTIPKSRCTDFQCKHSMKYRLSFCRKTCGTC

SEQ ID NO: 155 - protein, 35 aa, Artificial Sequence
  ShK-Based Peptide
RSCIDTIPKSRCTDFQCKHSMAYRLSFCRKTCGTC

SEQ ID NO: 156 - protein, 34 aa, Artificial Sequence
  ShK-Based Peptide
SCIDTIPKSRCTDFQCKHSMKYRLSFCRKTCGTC

SEQ ID NO: 157 - protein, 34 aa, Artificial Sequence
  ShK-Based Peptide
SCIDTIPKSRCTDFQCKHSMAYRLSFCRKTCGTC

SEQ ID NO: 158 - protein, 35 aa, Artificial Sequence
  ShK-Based Peptide
RSCIDTIPKSRCTAAQCKHSMAYRLSFCRKTCGTC

SEQ ID NO: 159 - protein, 34 aa, Artificial Sequence
  ShK-Based Peptide
SCIDTIPKSRCTAAQCKHSMAYRLSFCRKTCGTC

SEQ ID NO: 160 - protein, 35 aa, Artificial Sequence
  ShK-Based Peptide
RSCIDTIPKSRCTAIQCKHSMKYRLSFCRKTCGTC

SEQ ID NO: 161 - protein, 35 aa, Artificial Sequence
  ShK-Based Peptide
RSCIDTIPKSRCTAIQCKHSMAYRLSFCRKTCGTC

SEQ ID NO: 162 - protein, 34 aa, Artificial Sequence
  ShK-Based Peptide
SCIDTIPKSRCTAIQCKHSMKYRLSFCRKTCGTC

SEQ ID NO: 163 - protein, 34 aa, Artificial Sequence
  ShK-Based Peptide
SCIDTIPKSRCTAIQCKHSMAYRLSFCRKTCGTC

SEQ ID NO: 164 - protein, 35 aa, Artificial Sequence
  ShK-Based Peptide
RSCIDTIPKSRCTAVQCKHSMKYRLSFCRKTCGTC

SEQ ID NO: 165 - protein, 35 aa, Artificial Sequence
  ShK-Based Peptide
RSCIDTIPKSRCTAVQCKHSMAYRLSFCRKTCGTC

SEQ ID NO: 166 - protein, 34 aa, Artificial Sequence
  ShK-Based Peptide
SCIDTIPKSRCTAVQCKHSMKYRLSFCRKTCGTC

SEQ ID NO: 167 - protein, 34 aa, Artificial Sequence
  ShK-Based Peptide
SCIDTIPKSRCTAVQCKHSMAYRLSFCRKTCGTC

SEQ ID NO: 168 - protein, 35 aa, Artificial Sequence
  ShK-Based Peptide
RSCIDTIPKSRCTAFRCKHSMKYRLSFCRKTCGTC

SEQ ID NO: 169 - protein, 35 aa, Artificial Sequence
  ShK-Based Peptide
RSCIDTIPKSRCTAFRCKHSMAYRLSFCRKTCGTC

SEQ ID NO: 170 - protein, 34 aa, Artificial Sequence
  ShK-Based Peptide
SCIDTIPKSRCTAFRCKHSMKYRLSFCRKTCGTC

SEQ ID NO: 171 - protein, 34 aa, Artificial Sequence
  ShK-Based Peptide
SCIDTIPKSRCTAFRCKHSMAYRLSFCRKTCGTC

SEQ ID NO: 172 - protein, 35 aa, Artificial Sequence
  ShK-Based Peptide
RSCIDTIPKSRCTAFKCKHSMKYRLSFCRKTCGTC

SEQ ID NO: 173 - protein, 35 aa, Artificial Sequence
  ShK-Based Peptide
RSCIDTIPKSRCTAFKCKHSMAYRLSFCRKTCGTC

SEQ ID NO: 174 - protein, 34 aa, Artificial Sequence
  ShK-Based Peptide
SCIDTIPKSRCTAFKCKHSMKYRLSFCRKTCGTC

SEQ ID NO: 175 - protein, 34 aa, Artificial Sequence
  ShK-Based Peptide
SCIDTIPKSRCTAFKCKHSMAYRLSFCRKTCGTC

SEQ ID NO: 176 - protein, 35 aa, Artificial Sequence
  ShK-Based Peptide
RSCIDTIPASECTAFQCKHSMKYRLSFCRKTCGTC

SEQ ID NO: 177 - protein, 35 aa, Artificial Sequence
  ShK-Based Peptide
RSCIDTIPASECTAFQCKHSMAYRLSFCRKTCGTC

SEQ ID NO: 178 - protein, 34 aa, Artificial Sequence
  ShK-Based Peptide
SCIDTIPASECTAFQCKHSMKYRLSFCRKTCGTC

SEQ ID NO: 179 - protein, 34 aa, Artificial Sequence
  ShK-Based Peptide
SCIDTIPASECTAFQCKHSMAYRLSFCRKTCGTC

SEQ ID NO: 180 - protein, 35 aa, Artificial Sequence
  ShK-Based Peptide
RSCIDTIPVSECTAFQCKHSMKYRLSFCRKTCGTC

SEQ ID NO: 181 - protein, 35 aa, Artificial Sequence
  ShK-Based Peptide
RSCIDTIPVSECTAFQCKHSMAYRLSFCRKTCGTC

SEQ ID NO: 182 - protein, 34 aa, Artificial Sequence
  ShK-Based Peptide
SCIDTIPVSECTAFQCKHSMKYRLSFCRKTCGTC

SEQ ID NO: 183 - protein, 34 aa, Artificial Sequence
  ShK-Based Peptide
SCIDTIPVSECTAFQCKHSMAYRLSFCRKTCGTC

SEQ ID NO: 184 - protein, 35 aa, Artificial Sequence
  ShK-Based Peptide
RSCIDTIPVSACTAFQCKHSMKYRLSFCRKTCGTC

SEQ ID NO: 185 - protein, 35 aa, Artificial Sequence
  ShK-Based Peptide
RSCIDTIPVSACTAFQCKHSMAYRLSFCRKTCGTC

SEQ ID NO: 186 - protein, 34 aa, Artificial Sequence
  ShK-Based Peptide
SCIDTIPVSACTAFQCKHSMKYRLSFCRKTCGTC

SEQ ID NO: 187 - protein, 34 aa, Artificial Sequence
  ShK-Based Peptide
SCIDTIPVSACTAFQCKHSMAYRLSFCRKTCGTC

SEQ ID NO: 188 - protein, 35 aa, Artificial Sequence
  ShK-Based Peptide
RSCIDTIPASACTAFQCKHSMKYRLSFCRKTCGTC

SEQ ID NO: 189 - protein, 35 aa, Artificial Sequence
  ShK-Based Peptide
RSCIDTIPASACTAFQCKHSMAYRLSFCRKTCGTC

SEQ ID NO: 190 - protein, 34 aa, Artificial Sequence
  ShK-Based Peptide
SCIDTIPASACTAFQCKHSMKYRLSFCRKTCGTC

SEQ ID NO: 191 - protein, 34 aa, Artificial Sequence
  ShK-Based Peptide
SCIDTIPASACTAFQCKHSMAYRLSFCRKTCGTC

SEQ ID NO: 192 - protein, 35 aa, Artificial Sequence
  ShK-Based Peptide
RSCIDTIPKSECTDIRCKHSMKYRLSFCRKTCGTC

SEQ ID NO: 193 - protein, 35 aa, Artificial Sequence
  ShK-Based Peptide
RSCIDTIPKSECTDIRCKHSMAYRLSFCRKTCGTC

SEQ ID NO: 194 - protein, 34 aa, Artificial Sequence
  ShK-Based Peptide
SCIDTIPKSECTDIRCKHSMKYRLSFCRKTCGTC

SEQ ID NO: 195 - protein, 34 aa, Artificial Sequence
  ShK-Based Peptide
SCIDTIPKSECTDIRCKHSMAYRLSFCRKTCGTC

SEQ ID NO: 196 - protein, 35 aa, Artificial Sequence
  ShK-Based Peptide
RSCIDTIPVSECTDIRCKHSMKYRLSFCRKTCGTC

SEQ ID NO: 197 - protein, 35 aa, Artificial Sequence
  ShK-Based Peptide
RSCIDTIPVSECTDIRCKHSMAYRLSFCRKTCGTC

SEQ ID NO: 198 - protein, 34 aa, Artificial Sequence
  ShK-Based Peptide
SCIDTIPVSECTDIRCKHSMKYRLSFCRKTCGTC

SEQ ID NO: 199 - protein, 34 aa, Artificial Sequence
  ShK-Based Peptide
SCIDTIPVSECTDIRCKHSMAYRLSFCRKTCGTC

SEQ ID NO: 200 - protein, 35 aa, Artificial Sequence
  ShK-Based Peptide
RSCIDTIPVSECTDIQCKHSMKYRLSFCRKTCGTC

SEQ ID NO: 201 - protein, 35 aa, Artificial Sequence
  ShK-Based Peptide
RSCIDTIPVSECTDIQCKHSMAYRLSFCRKTCGTC

SEQ ID NO: 202 - protein, 34 aa, Artificial Sequence
  ShK-Based Peptide
SCIDTIPVSECTDIQCKHSMKYRLSFCRKTCGTC

SEQ ID NO: 203 - protein, 34 aa, Artificial Sequence
  ShK-Based Peptide
SCIDTIPVSECTDIQCKHSMAYRLSFCRKTCGTC

SEQ ID NO: 204 - protein, 35 aa, Artificial Sequence
  ShK-Based Peptide
RTCKDLIPVSECTDIRCKHSMKYRLSFCRKTCGTC

SEQ ID NO: 205 - protein, 35 aa, Artificial Sequence
  ShK-Based Peptide
RTCKDLIPVSECTDIRCKHSMAYRLSFCRKTCGTC

SEQ ID NO: 206 - protein, 34 aa, Artificial Sequence
  ShK-Based Peptide
TCKDLIPVSECTDIRCKHSMKYRLSFCRKTCGTC

SEQ ID NO: 207 - protein, 34 aa, Artificial Sequence
  ShK-Based Peptide
TCKDLIPVSECTDIRCKHSMAYRLSFCRKTCGTC

SEQ ID NO: 208 - protein, 37 aa, Artificial Sequence
  ShK-Based Peptide
XXRSCIDTIPKSRCTAFQCKHSMKYRLSFCRKTCGTC

SEQ ID NO: 209 - protein, 37 aa, Artificial Sequence; identical to SEQ ID NO: 208
  ShK-Based Peptide

SEQ ID NO: 210 - protein, 37 aa, Artificial Sequence; identical to SEQ ID NO: 208
  ShK-Based Peptide

SEQ ID NO: 211 - protein, 35 aa, Artificial Sequence
  ShK-Based Peptide
QSCADTIPKSRCTAAQCKHSMKYRLSFCRKTCGTC

SEQ ID NO: 212 - protein, 35 aa, Artificial Sequence
  ShK-Based Peptide
QSCADTIPKSRCTAAQCKHSMAYRLSFCRKTCGTC

SEQ ID NO: 213 - protein, 35 aa, Artificial Sequence
  ShK-Based Peptide
QSCADTIPKSRCTAAQCKHSMXYRLSFCRKTCGTC

SEQ ID NO: 214 - protein, 35 aa, Artificial Sequence
  ShK-Based Peptide
QSCADTIPKSRCTAAQCKHSMKYRASFCRKTCGTC

SEQ ID NO: 215 - protein, 35 aa, Artificial Sequence
  ShK-Based Peptide
QSCADTIPKSRCTAAQCKHSMAYRASFCRKTCGTC

SEQ ID NO: 216 - protein, 35 aa, Artificial Sequence
  ShK-Based Peptide
QSCADTIPKSRCTAAQCKHSMXYRASFCRKTCGTC

SEQ ID NO: 217 - protein, 37 aa, Artificial Sequence; identical to SEQ ID NO: 208
  ShK-Based Peptide

SEQ ID NO: 218 - protein, 37 aa, Artificial Sequence
  ShK-Based Peptide
XXRSCIDTIPKSRCTAFQCKHSXKYRLSFCRKTCGTC

SEQ ID NO: 219 - protein, 37 aa, Artificial Sequence; identical to SEQ ID NO: 208
  ShK-Based Peptide

SEQ ID NO: 220 - protein, 37 aa, Artificial Sequence; identical to SEQ ID NO: 218
  ShK-Based Peptide

SEQ ID NO: 221 - protein, 39 aa, Artificial Sequence
  ShK-Based Peptide
XXXXRSCIDTIPKSRCTAFQCKHSMKYRLSFCRKTCGTC

SEQ ID NO: 222 - protein, 37 aa, Artificial Sequence
  ShK-Based Peptide
XXRSCIDTIPKSRCTAFKCKHSXKYRLSFCRKTCGTC

SEQ ID NO: 223 - protein, 37 aa, Artificial Sequence; identical to SEQ ID NO: 208
  ShK-Based Peptide

SEQ ID NO: 224 - protein, 35 aa, Artificial Sequence
  ShK-Based Peptide
RSCIDTIPKSRCTAFQCKHSXXYRLSFCRKTCGTC

SEQ ID NO: 225 - protein, 35 aa, Pandinus imperator
LVKCRGTSDCGRPCQQQTGCPNSKCINRMCKCYGC

SEQ ID NO: 226 - protein, 35 aa, Pandinus imperator
TISCTNPKQCYPHCKKETGYPNAKCMNRKCKCFGR

SEQ ID NO: 227 - protein, 35 aa, Pandinus imperator
TISCTNEKQCYPHCKKETGYPNAKCMNRKCKCFGR

SEQ ID NO: 228 - protein, 38 aa, Pandinus imperator
IEAIRCGGSRDCYRPCQKRTGCPNAKCINKTCKCYGCS

SEQ ID NO: 229 - protein, 34 aa, Heterometrus spinifer
ASCRTPKDCADPCRKETGCPYGKCMNRKCKCNRC

SEQ ID NO: 230 - protein, 38 aa, Leiurus quinquestriatus hebraeus
GVPINVSCTGSPQCIKPCKDAGMRFGKCMNRKCHCTPK

SEQ ID NO: 231 - protein, 38 aa, Leiurus quinquestriatus hebraeus
GVPINVKCTGSPQCLKPCKDAGMRFGKCINGKCHCTPK

SEQ ID NO: 232 - protein, 38 aa, Orthochirus scrobiculosus
GVIINVKCKISRQCLEPCKKAGMRFGKCMNGKCHCTPK

SEQ ID NO: 233 - protein, 35 aa, Anuroctonus phaiodactylus
ZKECTGPQHCTNFCRKNKCTHGKCMNRKCKCFNCK

SEQ ID NO: 234 - protein, 39 aa, Centruroides noxius
TIINVKCTSPKQCSKPCKELYGSSAGAKCMNGKCKCYNN

SEQ ID NO: 235 - protein, 39 aa, Centruroides limbatus
TVIDVKCTSPKQCLPPCKAQFGLRAGAKCMNGKCKCYPH

SEQ ID NO: 236 - protein, 37 aa, Leiurus quinquestriatus hebraeus
QFTNVSCTTSKECWSVCQRLHNTSRGKCMNKKCRCYS

SEQ ID NO: 237 - protein, 37 aa, Tityus serrulatus
VFINAKCRGSPECLPKCKEAIGKAAGKCMNGKCKCYP

SEQ ID NO: 238 - protein, 37 aa, Bunodosoma granuliferum
VCRDWFKETACRHAKSLGNCRTSQKYRANCAKTCELC

SEQ ID NO: 239 - protein, 38 aa, Mesobuthus martensii
VGINVKCKHSGQCLKPCKDAGMRFGKCINGKCDCTPKG

SEQ ID NO: 240 - protein, 37 aa, Mesobuthus martensii
QFTDVKCTGSKQCWPVCKQMFGKPNGKCMNGKCRCYS

SEQ ID NO: 241 - protein, 37 aa, Tityus cambridgei
VFINVKCRGSKECLPACKAAVGKAAGKCMNGKCKCYP

SEQ ID NO: 242 - protein, 35 aa, Tityus cambridgei
TGPQTTCQAAMCEAGCKGLGKSMESCQGDTCKCKA

SEQ ID NO: 243 - protein, 36 aa, Vaejovis mexicanus smithi
  MOD_RES (36)..(36): AMIDATION
AAAISCVGSPECPPKCRAQGCKNGKCMNRKCKCYYC

SEQ ID NO: 244 - protein, 35 aa, Heteractis magnifica
RTCKDLIPVSECTDIRCRTSMKYRLNLCRKTCGSC

SEQ ID NO: 245 - protein, 36 aa, Actinia equina
GCKDNFSANTCKHVKANNNCGSQKYATNCAKTCGKC

SEQ ID NO: 246 - protein, 36 aa, Anemonia sulcata
ACKDNFAAATCKHVKENKNCGSQKYATNCAKTCGKC

SEQ ID NO: 247 - protein, 39 aa, Centruroides margaritatus
TIINVKCTSPKQCLPPCKAQFGQSAGAKCMNGKCKCYPH

SEQ ID NO: 248 - protein, 38 aa, Androctonus mauritanicus mauritanicus
GVEINVKCSGSPQCLKPCKDAGMRFGKCMNRKCHCTPK

SEQ ID NO: 249 - protein, 37 aa, Androctonus australis
VRIPVSCKHSGQCLKPCKDAGMRFGKCMNGKCDCTPK

SEQ ID NO: 250 - protein, 34 aa, Scorpio maurus palmatus
VSCTGSKDCYAPCRKQTGCPNAKCINKSCKCYGC

SEQ ID NO: 251 - protein, 36 aa, Mesobuthus tamulus
QFTDVDCSVSKECWSVCKDLFGVDRGKCMGKKCRCY

SEQ ID NO: 252 - protein, 38 aa, Odontobuthus doriae
GVPTDVKCRGSPQCIQPCKDAGMRFGKCMNGKCHCTPK

SEQ ID NO: 253 - protein, 38 aa, Mesobuthus tamulus sindicus
GVPINVKCRGSPQCIQPCRDAGMRFGKCMNGKCHCTPQ

SEQ ID NO: 254 - protein, 37 aa, Buthus occitanus israelis
GVPINVKCRGSRDCLDPCKKAGMRFGKCINSKCHCTP

SEQ ID NO: 255 - protein, 38 aa, Leiurus quinquestriatus hebraeus
GVPINVPCTGSPQCIKPCKDAGMRFGKCMNRKCHCTPK

SEQ ID NO: 256 - protein, 37 aa, Buthus occitanus tunetanus
VGIPVSCKHSGQCIKPCKDAGMRFGKCMNRKCDCTPK

SEQ ID NO: 257 - protein, 37 aa, Artificial Sequence; identical to SEQ ID NO: 218
  ShK-Based Protein

SEQ ID NO: 258 - protein, 37 aa, Artificial Sequence; identical to SEQ ID NO: 218
  ShK-Based Protein

SEQ ID NO: 259 - protein, 37 aa, Artificial Sequence; identical to SEQ ID NO: 218
  ShK-Based Protein

SEQ ID NO: 260 - protein, 37 aa, Artificial Sequence; identical to SEQ ID NO: 218
  ShK-Based Protein

(19) United States
(12) Patent Application Publication
(10) Pub. No.: US 2016/0338967 A1
(43) Pub. Date: Nov. 24, 2016

(54) SUSTAINED RELEASE DEPOT FORMULATIONS OF THERAPEUTIC PROTEINS, AND USES THEREOF

(71) Applicant: Kineta One, LLC, Seattle, WA (US)

(72) Inventors: Shawn P. IADONATO, Seattle, WA (US); Ernesto J. MUNOZ, Seattle, WA (US); James CHESKO, Seattle, WA (US); Eric J. TARCHA, Seattle, WA (US)

(73) Assignee: KINETA ONE, LLC, Seattle, WA (US)

(21) Appl. No.: 15/107,355

(22) PCT Filed: Dec. 23, 2014
(86) PCT No.: PCT/US2014/072253

§ 371 Date: Jun. 22, 2016

Related U.S. Application Data
(60) Provisional application No. 61/920,383, filed on Dec. 23, 2013.

(51) Int. Cl. A61K 9/50; A61K 38/17; A61K 47/26; A61K 47/32

(52) U.S. Cl. CPC A61K 9/5031; A61K 47/32; A61K 38/1767; A61K 47/26

(57) ABSTRACT

Depot formulations including therapeutic proteins are provided. The therapeutic proteins can be toxin-based therapeutic proteins. The depot formulations release the therapeutic protein within sustained effective levels for at least one month following a single administration. The toxin-based therapeutic proteins can include ShK-based proteins.

### CROSS-REFERENCE TO RELATED APPLICATION

This application claims priority to, and the benefit of, U.S. Provisional Patent Application No. 61/920,383, filed Dec. 23, 2013, the entire contents of which are incorporated by reference herein.

### FIELD OF THE DISCLOSURE

The disclosure relates to formulations for the sustained release of therapeutic proteins, including toxin-based therapeutic proteins with at least one disulfide bridge. Methods of creating and using the formulations are also disclosed. Following a single administration, the formulations achieve sustained effective levels of the therapeutic protein in a subject for at least one month.

### BACKGROUND OF THE DISCLOSURE

Generally therapeutic medicines possess a range of acceptable effective levels between a minimum effective dose and maximum tolerable dose known as the therapeutic window of the medicine. Maintenance of the medicine within the therapeutic window requires sustained, effective levels of the medicine without exceeding the maximum tolerable dose.

In the case of therapeutic proteins, achieving sustained effective levels without exceeding the maximum tolerable dose is often accomplished through frequent parenteral injections. The need for frequent injections, however, is inconvenient, can lead to poor subject compliance, and can cause fluctuating and potentially deleterious levels of the therapeutic protein in the subject.

An alternative approach to multiple injections is utilizing biodegradable materials that modulate the release of therapeutic proteins over time once administered to a subject. Through the process of encapsulation and the creation of molecular arrangements whose interactions lead to a controlled slow release of a therapeutic protein, sustained elevated levels of the protein theoretically can be achieved. While the described approach works well in theory, it has been found that such release systems often show a very high initial rate of release called a “burst” that allows a large amount of the therapeutic protein to escape quickly following administration. This burst can create protein concentrations that exceed the therapeutic window often causing unwanted side effects and subsequently leaving insufficient quantities in the release system to sustain effective levels later in time. Moreover, such compositions often generate unwanted peaks and troughs in blood concentration leading to inconsistent treatment levels over time.

Factors such as physiological temperatures, the milieu of biomolecules, and the immune response to the administration of controlled release compositions can unfavorably alter the disposition of the therapeutic protein through mechanisms, such as degradation and aggregation that contribute to poor bioavailability. These natural processes can interfere with the desired release profile and effectiveness of the composition, especially because proteins are inherently labile molecules with numerous defined pathways for degradation and elimination.

Controlled release compositions including gonadotropin releasing hormone (GnRH) agonists such as leuprolide (and related compounds including buserelin, histrelin, goserelin, nafarelin, and triptorellin) exhibiting sustained release for 1-3 months following administration have been successfully created (U.S. Pat. Nos. 5,980,945; 6,036,976; 6,337,618). Leutenizing hormone releasing hormone (LH-RH) agonists have also been combined into controlled release compositions (U.S. Pat. Nos. 3,853,837, 4,008,209, 3,972,859, 4,086,219, 4,124,577, 4,253,997, and 4,317,815; Great Britain Patent No. 1423083). The active compounds in these formulations, however, are very short (˜9 amino acids) and lack higher order structural elements often associated with therapeutic protein activity such as secondary structural elements (such as α-helices and β-sheets), and tertiary structural elements. For example, NMR structures have suggested at most a type I or type II β turn. Additionally, structural components appear not to be necessary for therapeutic efficacy of these compounds as simple linear analogs show potent activity against relevant targets such as MCF-7 breast cancer line cells.

Much work has been done in an attempt to address problems associated with creating sustained release depot formulations of more complex therapeutic proteins (see, for example, Allison, Expert Opin. Drug Deliv. 5:615-28, 2008; Xu, et al., Acta Pharmaceutica Sinica, 42:1-7, 2007; Jung, et al., Arch. Pharm. Res. 32:359-65, 2009; Bouissou, et al., Pharm. Res. 23:1295-305, 2006; Luan, et al., Eur. J. Pharm. Biopharm. 63:205-14, 2006; Duncan, et al., J. Control Release 110:34-48, 2005; Leach, et al., J. Pharm. Sci. 94:56-69, 2005; Yeo, et al., Arch. Pharm. Res. 27:1-12, 2004; Yeh, et al., J. Microencapsul. 24:82-93, 2007; Costantino, et al., J. Pharm. Sci. 93:2624-2634, 2004; Yeo, et al., Arch Pharm Res., 27:1-12, 2004; Pean, et al., J. Control Release 56:175-187, 1998; U.S. Patent No. 5,891,478; Carrasquillo, J. Control Release 76:199-208, 2001; Castellanos, et al., J. Pharm Pharmacol 53:1099-1107, 2001; Lee, et al., J. Biol. Chem. 256:7193-7201, 1981; Perez. et al., J. Pharm Pharmacol 54:301-313, 2002; and Taylor, et al., Diabetes 51 (Suppl 2):A85, 2002). Despite significant work in this area, however, only a handful of peptides have successfully been formulated into controlled release compositions.

Prior examples of attempting to control and lower initial bursts of protein release have largely been unsuccessful and include: bovine serum albumin (BSA) (Samadi, et al., Biomacromolecules 14:1044-53 2013); α-chymotrypsin (Flores-Fernández, et al., Results Pharma, Sci. 2:46-51, 2012); granulocyte macrophage colony stimulating factor (GM-CSF) (Zheng, et al., J. Microencapsul. 28:743-51, 2011); interferon α-2b (Li, et al., Int. J. Pharm. 410:48-53, 2011); tissue-necrosis factor α (TNF-α) (Kim, et al., J. Control Release. 150:63-9, 2011); erythropoietin, nerve growth factor, and human growth factor (Ye, et al., J Control Release. 146:241-60, 2010), and human growth hormone (U.S. Pat. No. 5,891,478). Formulations exhibiting pronounced early bursts have also been reviewed in, for example, Zheng et al., Drug Deliv. 17:77-82, 2010.

Additional challenges associated with achieving the sustained release of therapeutic proteins include instability of the encapsulated protein and/or incomplete release of the therapeutic protein from the composition (Yeo, et al., Arch Pharm Res, 27:1-12, 2004). Many approaches have been attempted to address each of these issues, including stabilizing excipients (Lee, et al., J. Biol. Chem., 256:7193-7201, 1981), core-shell structures (Yuan et al., Int. J. Nanomedicine 7:257-270, 2012), and molecular engineering of the active components (Lucke, et al., Pharm. Res., 19:175-181, 2002). Despite all of these approaches and attempts, therapeutic proteins have largely evaded successful formulation into sustained release systems. See, for example, Pai, et al., AAPS J. 11(1): 88-98, March 2009; PMCID: PMC2664882.

### SUMMARY OF THE DISCLOSURE

A need exists for the ability to administer therapeutic proteins in a form that achieves sustained release of effective levels of the protein without requiring multiple injections. A need especially exists for the ability to administer therapeutic proteins that are more complex than previously formulated short proteins lacking complex structures such as GnRH agonists or LH-RH.

The present disclosure addresses these needs by providing formulations for the sustained release of therapeutic proteins including toxin-based therapeutic proteins. The proteins can include at least one disulfide bridge. The formulations provide sustained effective levels of the therapeutic protein for at least one month following a single administration. Methods of creating and using the formulations are also disclosed.

Particularly, the present disclosure provides sustained release depot formulations including at least one therapeutic protein. More particularly, the sustained release depot formulations include an internal aqueous phase including a therapeutic protein, a second phase including a polymer that can be biodegradable (oil/solid phase), and a third, external aqueous phase in which particles are dispersed. The internal aqueous phase can be a specifically chemically modified microenvironment in which the pH, salt concentration, solvent, stabilizers, and release modifiers are chosen to retain the native and active conformation of the particular therapeutic protein and to allow its compatibility with the second phase polymer so as to achieve sustained release of the protein over time.

### BRIEF DESCRIPTION OF THE DRAWINGS

FIG. 1 is a plot of in vitro release of formulations with polymer types PLG1A, PLG2A, PLG3A, PLG5E, and PLG7E (representing a range of poly(lactide-co-glycolides) (PLG) of different molecular weights and end capped chemistries) into an aqueous 2% (w/v) sodium dodecyl sulfate (SDDS) medium at 37° C. with mechanical agitation.

FIGS. 2A and 2B show the dispersion size for three separate batches of PLG2A formulations, as measured by dynamic light scattering. FIG. 2A shows size distributions of ShK-186 sustained release depot formulations plotted by intensity; FIG. 2B shows the volume weighted distribution of particles in the formulation as measured by dynamic light scattering.

FIG. 3 is a measurement of the zeta potential (particle surface charge, as measured by electrophoretic mobility) for therapeutic protein-loaded PLG2A polymer formulations. The anionic surface layers help confer stability of the dispersion in aqueous suspensions. Zeta potential measurements showed similar, tight clustering of anionic particles with charges of −75, −72 and −72 mV, providing coulombic interactions that contribute to colloidal stability through electrostatic repulsion.

FIG. 4 is an optical microscope image of a formulation (PLG2A), to show the shape of the particles within the formulation and approximate uniform, geometric dimensions. The figure shows an optical microscopic (100×) image of PLG polymer encapsulating ShK-186, showing round (presumably spherical) particles with a size of one micrometer.

FIGS. 5A and 5B show the in vivo release rate of ShK-186 dosed at 40,000 μg/kg following a single subcutaneous (SC) injection of various formulations into Sprague-Dawley rats. Suspensions were made with PLG1A, PLG2A and PLG3A. FIG. 5A shows this data in a linear scale while FIG. 5B presents a logarithmic (Log) scale.

FIGS. 6A and B are plots of in vivo release for different doses (high=40,000 μg/kg, medium=20,000 μg/kg, low=10,000 μg/kg) of ShK-186 formulated with PLG2A. The blood serum levels of ShK-186 are maintained for more than 30 days over a relatively narrow range of concentrations in Sprague-Dawley rats. FIG. 6A shows a linear scale. FIG. 6B shows a Log scale.

FIGS. 7A and 7B are plots of in vivo release for ShK-192 dosed at 10,000 μg/kg in Sprague Dawley rats, following a single SC injection. The maximum concentration reached was near t=12 days suggesting a gradual release of the therapeutic protein from the evolving PLG2A sustained release depot formulation, a process that continues relatively smoothly for over 30 days. FIG. 7A shows a linear scale. FIG. 7B shows a Log scale.

FIG. 8 is a graph of in vivo release of ShK-186 dosed with one SC injection at 20,000 μg/kg in Sprague-Dawley rats for a PLG2A formulation, showing the Cₘₐₓ at t=2 days and a long, sustained release tail extending out well over 56 days.

### DETAILED DESCRIPTION

Many proteins are useful as therapeutic medicines because of the biological activity they exhibit in vivo. However, in attempting to use these proteins as therapeutics it has been difficult to achieve sustained effective levels of the protein without frequent administration through injection.

An alternative approach to multiple injections is administering therapeutic proteins that have been encapsulated in biodegradable materials that modulate the release of the therapeutic proteins over time. While this approach functions well in theory, it has been found that such controlled release compositions are difficult to achieve. For example, many such controlled release compositions show a burst which can create drug concentrations that exceed the therapeutic window, thereby increasing the potential for unwanted side effects, and ultimately leaving insufficient quantities of the therapeutic protein in the composition to sustain effective levels later in time. Factors such as physiological temperatures, the milieu of biomolecules, and the immune response to the administration of controlled release compositions, can also unfavorably alter the disposition of therapeutic proteins.

The sustained release depot formulations disclosed herein overcome the failures of previous attempts, such as those described in the references cited herein. The formulations disclosed herein can increase patient compliance through ease of dosing (avoidance of multiple injections) and reduction of unwanted side effects.

In particular embodiments, the formulations disclosed herein show release profiles with minimal burst effects and ratios of Cₘₐₓ to C_{average} that equal less than five or less than three. These ratios suggest the successful combination of synergistic aspects of molecular structure, formulation interactions, and processes to achieve a relatively uniform release of stabilized therapeutic proteins as measured both in vitro and in vivo using structure sensitive bioanalytics. Characteristics of the release profile including the size of the second maxima (triphasic component, Luan, et al., Eur. J. Pharm. Biopharm. 63:205-14, 2006) relative to the first and the depth of the trough have been controlled to produce blood serum levels that are remarkably steady over extended time periods, e.g., greater than one month and up to at least 56 days. Accordingly, the sustained release depot formulations disclosed herein achieve suitable release profiles of therapeutic proteins (such as near zero-order release kinetics) over a period of one month or greater following a single administration.

“Sustained release” should be interpreted to include: (1) release within effective levels for at least one month following a single administration; or (2) release within effective levels wherein the Cₘₐₓ to C_{average} ratio does not exceed five or does not exceed three for at least one month following a single administration. “Sustained release” can also be interpreted to include: (1) release within effective levels for at least 56 days following a single administration; or (2) release within effective levels wherein the Cₘₐₓ to C_{average} ratio does not exceed five or does not exceed three for at least 56 days following a single administration.

“Effective levels” are those within a particular protein's therapeutic window that achieve an intended prophylactic treatment or therapeutic treatment without the creation of unintended side effects.

“Depot formulations” include a therapeutic protein delivery systems that provides sustained release of the therapeutic protein into surrounding tissue following administration.

The described depot formulations are accomplished through the combination of therapeutic proteins and excipients in processes disclosed herein that create, in particular embodiments, microencapsulated, biodegradable particulate dispersions.

In particular embodiments, the present disclosure includes depot formulations including at least one therapeutic protein. More particularly, the depot formulations can include an internal aqueous phase including the therapeutic protein, a second middle phase including a polymer (oil/solid phase; in particular embodiments, the polymer can be biodegradable), and a third, external aqueous phase in which particles can be dispersed. The internal aqueous phase is a specifically chemically modified microenvironment in which the pH, salt concentration, solvent, stabilizers, and release modifiers are chosen to retain the native and active conformation of the particular therapeutic protein, and to allow its compatibility with the second polymer phase so as to achieve sustained release of the protein over time.

Embodiments disclosed herein can include: (i) an internal aqueous phase including a therapeutic protein, the therapeutic protein present at 0.025% weight/weight (w/w) to 5% w/w of the weight of the depot formulation; (ii) a polymer-based oil/solid phase; and (iii) an external aqueous phase including a surfactant present at 0.01% w/w to 1% w/w of the weight of the depot formulation, wherein the depot formulation provides sustained release of the therapeutic protein within effective levels for at least one month following a single administration.

In various embodiments, the depot formulation includes a particle made up of an internal aqueous phase and a polymer phase. In various embodiments, the depot formulation includes a particle made up of an internal aqueous phase and a polymer phase, which is surrounded by an aqueous phase.

POLYMERS. The specific polymer compositions and preparations used in the depot formulations disclosed herein provide a chemical microenvironment that under physiological conditions create a structure allowing the sustained release of a therapeutic protein from the structure. In particular embodiments, the sustained release occurs through processes such as diffusion through a hydrated polymer matrix.

The polymer can typically be only sparingly soluble or insoluble in water; as well as biocompatible and biodegradable following administration to a subject. “Sparingly soluble” means that the polymer is no more than 3% w/w soluble in water. The average molecular weight of polymers used in the depot formulations disclosed herein is generally in the range of 3,000 Daltons (Da) to 100,000 Da, and in particular embodiments, around 3,000 to 20,000 Da. The polydispersity of these polymers typically ranges from 1.1 to 4.0. The amount of a biocompatible polymer used in a particular depot formulations depends on the strength of pharmacological activity of the therapeutic protein and the desired rate of its release.

The chemical nature of the polymer can include acids, aliphatic polyesters (homopolymers such as poly(lactic acid)), copolymers such as poly(lactide-co-glycolide), hydroxycarboxylic acids, alpha-hydroxy acids, poly(amino acids), and/or poly(cyanoacrylic) esters. The most preferred are esters of lactic and/or glycolic acid, i.e. poly(lactides), poly(glycolides), and/or PLG. Depot formulations disclosed herein may also include pegylated, ethoxylated and other derivatized versions of these polymers, including hydrophilic (carboxyl-terminated) and more hydrophobic (ester-terminated) end capped structures.

In particular embodimients, exemplary polymers include biodegradable polymers including poly(lactide), poly(glycolide), poly(caprolactone), and poly(lactide)-co(glycolide) (PLG) of desirable lactide:glycolide ratios, average molecular weights, polydispersities, and terminal group chemistries.

In various embodiments, the polymer used can be a carboxy-terminated medium molecular weight PLG. “Low molecular weight” refers to polymers having a molecular weight of 1,000 Da to 10,000 Da. “Medium molecular weight” refers to polymers having a molecular weight of 10,000 Da to 25,000 Da. “High molecular weight” refers to polymers having a molecular weight greater than 25,000 Da.

In particular embodiments, the polymer used can be PLG1A, PLG2A, PLG3A, PLG5E, or PLG7E. PLG1A is a carboxy-terminated PLG polymer with a molecular weight of 5.9 kDa. PLG2A is a carboxy-terminated PLG polymer with a molecular weight of 13.6 kDa. PLG3A is a carboxy-terminated PLG polymer with a molecular weight of 32 kDa. PLG5E is an esterified PLG polymer with a molecular weight of 75 kDa. PLG7E is an esterified PLG polymer with a molecular weight of 68 kDa. Blending different polymer types in different ratios using various grades can result in characteristics that borrow from each of the contributing polymers. Accordingly, blends and co-polymers may also be used. Blends include mixtures of different polymers. Co-polymers include those made up of at least two different constituent monomers.

SOLVENTS & EXCIPIENTS. A highly acidic microenvironment created through the hydrolysis of ester linkages of poly(D,L-lactide-co-glycolide)s presents a harsh environment that can negatively affect the stability of therapeutic proteins. To overcome these challenges the internal aqueous phase of the depot formulations disclosed herein can be pH-controlled by buffer and salt solutions. The internal aqueous phase can be stabilized by a combination of pH buffer species and excipients including acetic acid, carbonic acid, phosphoric acid, and salts such as sodium hydrogen phosphate, hydrochloric acid, sodium hydroxide, arginine, and lysine. In particular embodiments, a phosphate buffered aqueous solution with 140 mM sodium chloride salt is used. In particular embodiments, these buffering systems are designed to keep the pH of the internal aqueous phase of the depot formulation above 6.

In particular embodiments, the internal aqueous phase can have a pH of 6.0, 7.0, 7.4, or 8.0 (phosphate buffer); 5.0 or 6.0 (histidine); 4.5, 5.0. or 6.0 (citrate); 4.5, 5.0, or 5.5 (acetate), Mg(OH)2. The ionic strengths of the internal aqueous phase can be O-200 mM with NaCl, KCl, and/or CaCl₂.

The internal aqueous phase may also include a protein stabilizer such as albumin, gelatin, citric acid, sodium ethylenediamine tetrammonium acetate, dextrin, sodium hydrosulfate, polyols such poly(ethylene glycol), and/or a preservative such as p-hydroxytoluene, p-hydroxybenzoic acid esters (methylparaben, propylparaben), benzyl alcohol, chlorobutanol, and thimerosal.

The use of different solvents (e.g., dichloromethane, chloroform, ethyl acetate, triacetin, N-methyl pyrrolidone, tetrahydrofuran, phenol, or combinations thereof) can alter particle size and structure to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Release modifiers such as surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Del.), poly(vinyl alcohol) (PVA), Brij® (Croda Americas, Wilmington, Del.), sucrose acetate isobutyrate (SAIB), salts, and buffers can also change properties of therapeutic protein release from the depot formulations.

Excipients that partition into the external phase boundary of particles within the depot formulations such as surfactants including polysorbates, dioctylsulfosuccinates, poloxamers, and PVA, can also alter properties including particle stability and erosion rates, hydration and channel structure, interfacial transport, and kinetics in a favorable manner. The external phase boundary of the particles (which can be formed by, for example, the polymer solid/oil phase surrounding the internal aqueous phase) is the part of the particles adjacent to the external aqueous phase, which surrounds the particles.

In particular embodiments, the external aqueous phase can have a pH in the range of 5.5-8.5 and can include phosphate, citrate, and salts including NaCl, KCl, and/or CaCl2 in the O-200 mM range.

Different surfactant species may also be employed to stabilize the depot formulations disclosed herein as well as the described emulsions. Exemplary surfactants include ethylene-propylene oxide (PEO-PPO) di- and tri-block co-polymers, sorbitan esters such as Tween® (Croda Americas, Wilmington, Del.), Span®, PVA, Brij®, Eudragit® (Evonik Rohm GmbH, Darmstadt, Germany), poloxamers, docusate sodium, and SDDS.

Additional processing of the disclosed depot formulations can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle to produce very low moisture powders that reconstitute to similar size and performance characteristics of the original suspension can also be used.

THERAPEUTIC PROTEINS. Therapeutic proteins provided as part of the depot formulations described herein can include proteins that are longer in length and/or more structurally complex than those found in the previous controlled release compositions.

Exemplary therapeutic proteins are toxin-based therapeutic proteins. Particular examples of toxin-based therapeutic proteins for use in the depot formulations disclosed herein bind voltage gated channels. Exemplary voltage gated channels include Kv1.1, Kv1.2, Kv1.3, Kv1.4, Kv1.5, Kv1.6, Kv1.7, Kv2.1, Kv3.1, Kv3.2, Kv11.1, Kc1.1, Kc2.1, Kc3.1, Nav1.2, Nav1.4, and Cav1.2 channels.

Toxin proteins are produced by a variety of organisms and have evolved to bind to ion channels and receptors. Native toxin proteins from snakes, scorpions, spiders, bees, snails, and sea anemone are typically 10-80 amino acids in length and include 2 to 5 disulfide bridges that create compact molecular structures. These proteins appear to have evolved from a small number of structural frameworks. The proteins cluster into families of folding patterns that are conserved through cysteine/disulfide loop structures to maintain a three dimensional structure that contributes to potency, stability, and selectivity, all of which are elements of critical importance when creating the depot formulations of the present disclosure. (Pennington, et al., Biochemistry, 38, 14549-14558 (1999); Tudor, et al., Eur. J. Biochem., 251, 133-141 (1998); and Jaravine et al., Biochemistry, 36, 1223-1232, (1997)).

“Toxin-based therapeutic proteins” include toxin-based proteins of Table 1 (or a variant, D-substituted analog, carboxy-terminal amide, modification, derivative or pharmaceutically acceptable salt thereof), and ShK-based proteins of Table 2 (or a variant, D-substituted analog, carboxy-terminal amide, modification, derivative, or pharmaceutically acceptable salt thereof). Toxin-based therapeutic proteins can be synthetic or naturally-occurring.

“Toxin-based proteins” include any synthetic or naturally-known toxin protein and those proteins disclosed in Table 1, as well as variants, D-substituted analogs, carboxy-terminal amides, modifications, derivatives, and pharmaceutically acceptable salts thereof. Particular exemplary toxin-based therapeutic proteins for the depot formulations and use in the methods disclosed herein include the toxin-based proteins listed in Table 1, and as shown in the sequence listing as SEQ ID NO: 225-256.

TABLE 1
Exemplary Toxin-Based Proteins

| | Shorthand | SEQ |
|---|---|---|
| Sequence/Structure | ID | ID NO: |
| LVKCRGTSDCGRPCQQQTGCP | Pi1 | 225 |
| NSKCINRMCKCYGC | | |
| TISCTNPKQCYPHCKKETGYP | Pi2 | 226 |
| NAKCMNRKCKCFGR | | |
| TISCTNEKQCYPHCKKETGYP | Pi3 | 227 |
| NAKCMNRKCKCFGR | | |
| IEAIRCGGSRDCYRPCQKRTG | Pi4 | 228 |
| CPNAKCINKTCKCYGCS | | |
| ASCRTPKDCADPCRKETGCPY | HsTx1 | 229 |
| GKCMNRKCKCNRC | | |
| GVPINVSCTGSPQCIKPCKDA | AgTx2 | 230 |
| GMRFGKCMNRKCHCTPK | | |
| GVPINVKCTGSPQCLKPCKDA | AgTx1 | 231 |
| GMRFGKCINGKCHCTPK | | |
| GVIINVKCKISRQCLEPCKKA | OSK1 | 232 |
| GMRFGKCMNGKCHCTPK | | |
| ZKECTGPQHCTNFCRKNKCTH | Anuroctoxin | 232 |
| GKCMNRKCKCFNCK | | |
| TIINVKCTSPKQCSKPCKELY | NTx | 234 |
| GSSAGAKCMNGKCKCYNN | | |
| TVIDVKCTSPKQCLPPCKAQF | HgTx1 | 235 |
| GIRAGAKCMNGKCKCYPH | | |
| QFTNVSCTTSKECWSVCQRLH | ChTx | 236 |
| NTSRGKCMNKKCRCYS | | |
| VFINAKCRGSPECLPKCKEAI | Titystoxin-Ka | 237 |
| GKAAGKCMNGKCKCYP | | |
| VCRDWFKETACRHAKSLGNCR | BgK | 238 |
| TSQKYRANCAKTCELC | | |
| VGINVKCKHSGQCLKPCKDAG | BmKTx | 239 |
| MRFGKCINGKCDCTPKG | | |
| QFTDVKCTGSKQCWPVCKQMF | BmTx1 | 240 |
| GKPNGKCMNGKCRCYS | | |
| VFINVKCRGSKECLPACKAAV | Tc30 | 241 |
| GKAAGKCMNGKCKCYP | | |
| TGPQTTCQAAMCEAGCKGLGK | Tc32 | 242 |
| SMESCQGDTCKCKA | | |
| AAAISCVGSPECPPKCRAQGC | Vm24 | 243 |
| KNGKCMNRKCKCYYC-amide | | |
| RTCKDLIPVSECTDIRCRTSM | HmK | 244 |
| KYRLNLCRKTCGSC | | |
| GCKDNFSANTCKHVKANNNCG | Aek | 245 |
| SQKYATNCAKTCGKC | | |
| ACKDNFAAATCKHVKENKNCG | AsKS | 246 |
| SQKYATNCAKTCGKC | | |
| TIINVKCTSPKQCLPPCKAQF | MgTx | 247 |
| GQSAGAKCMNGKCKCYPH | | |
| GVEINVKCSGSPQCLKPCKDA | KTx1 | 248 |
| GMRFGKCMNRKCHCTPK | | |
| VRIPVSCKHSGQCLKPCKDAG | KTx2 | 249 |
| MRFGKCMNGKCDCTPK | | |
| VSCTGSKDCYAPCRKQTGCPN | MTx | 250 |
| AKCINKSCKCYGC | | |
| QFTDVDCSVSKECWSVCKDLF | IbTx | 251 |
| GVDRGKCMGKKCRCY | | |
| GVPTDVKCRGSPQCIQPCKDA | ODK2 | 252 |
| GMRFGKCMNGKCHCTPK | | |
| GVPINVKCRGSPQCIQPCRDA | Bs6 | 253 |
| GMRFGKCMNGKCHCTPQ | | |
| GVPINVKCRGSRDCLDPCKKA | BoiTx1 | 254 |
| GMRFGKCINSKCHCTP | | |
| GVPINVPCTGSPQCIKPCKDA | AgTx3 | 255 |
| GMRFGKCMNRKCHCTPK | | |
| VGIPVSCKHSGQCIKPCKDAG | KTx3 | 256 |
| MRFGKCMNRKCDCTPK | | |

ShK is a highly structured, 35 residue protein cross-linked by three disulfide bridges whose activity depends critically upon its three dimensional structure. A depot formulation that maintains potency of this therapeutic protein requires stabilization and retention of high order structural elements that were not necessary for or addressed in previous formulation attempts and hence provides improvements in therapeutic treatment.

ShK proteins are a subtype of toxin proteins that can be used in the depot formulations and methods disclosed herein. ShK proteins were originally isolated from the Caribbean sea anemone Stichodactyla helianthus. ShK proteins serve as inhibitors of Kv1.3 channels. By inhibiting Kv1.3 channels, ShK proteins can suppress activation, proliferation, and/or cytokine production of or by Effector memory cells (T_{EM}), in certain embodiments, at picomolar concentrations.

“Inhibitor” is any toxin-based therapeutic protein that decreases or eliminates a biological activity that normally results based on the interaction of a compound with a receptor including biosynthetic and/or catalytic activity, receptor, or signal transduction pathway activity, gene transcription or translation, cellular protein transport, etc.

A native ShK protein is described in, for example, Pennington, et al., Int. J. Pept. Protein Res., 46, 354-358 (1995). Exemplary ShK structures that are within the scope of the present disclosure are also published in Beeton, et al., Mol. Pharmacol., 67, 1369-1381 (2005); U.S. Publication No. 2008/0221024; PCT Publication No. WO/2012/170392; and in U.S. Pat. Nos. 8,080,523 and 8,440,621.

“ShK-based proteins” include any synthetic or naturally-known ShK proteins as well as variants, D-substituted analogs, carboxy-terminal amides, modifications, derivatives, and pharmaceutically acceptable salts thereof.

Particular exemplary ShK-based proteins for use in the depot formulations disclosed herein include those listed in Table 2, and as shown in the sequence listing as SEQ ID NO:1-224 and SEQ ID NO:257-260. ShK-based proteins utilized in particular embodiments disclosed herein include those of SEQ ID NO: 1, SEQ ID NO: 49, SEQ ID NO: 208, SEQ ID NO:257, SEQ ID NO:223, SEQ ID NO: 210, SEQ ID NO: 217, SEQ ID NO: 218, and SEQ ID NO: 221.

TABLE 2
Exemplary ShK-Based Proteins

| | | SEQ |
|---|---|---|
| Sequence/structure | Shorthand ID | ID NO: |
| RSCIDTIPKSRCTAFQCKHSMKYRLSFCRKTCGTC | ShK | 1 |
| RSCIDTIPKSRCTAFQSKHSMKYRLSFCRKTSGTC | ShK-S17/S32 | 2 |
| RSSIDTIPKSRCTAFQCKHSMKYRLSFCRKTCGTS | ShK-S3/S35 | 3 |
| SSCIDTIPKS RCTAFQCKHSMKYRLSFCRKTCGTC | ShK-S1 | 4 |
| (N-acetylR)SCIDTIPKSRCTAFQCKHSMKYRLSFCRKTCGTC | ShK-N-acetylarg1 | 5 |
| SCIDTIPKSRCTAFQCKHSMKYRLSFCRKTCGTC | ShK-d1 | 6 |
| CIDTIPKSRCTAFQCKHSMKYRLSFCRKTCGTC | ShK-d2 | 7 |
| ASCIDTIPKSRCTAFQCKHSMKYRLSFCRKTCGTC | ShK-A1 | 8 |
| QCIDTIPKSRCTAFQCKHSMKYRLSFCRKTCGTC | ShK-Q2 d1 | 9 |
| ACIDTIPKSRCTAFQCKHSMKYRLSFCRKTCGTC | ShK-A2 d1 | 10 |
| TCIDTIPKSRCTAFQCKHSMKYRLSFCRKTCGTC | ShK-T2 d1 | 11 |
| RQCIDTIPKSRCTAFQCKHSMKYRLSFCRKTCGTC | ShK-Q2 | 12 |
| RACIDTIPKSRCTAFQCKHSMKYRLSFCRKTCGTC | ShK-A2 | 13 |
| RTCIDTIPKSRCTAFQCKHSMKYRLSFCRKTCGTC | ShK-T2 | 14 |
| AQCIDTIPKSRCTAFQCKHSMKYRLSFCRKTCGTC | ShK-Q2 | 15 |
| AACIDTIPKSRCTAFQCKHSMKYRLSFCRKTCGTC | ShK-A1/A2 | 16 |
| ATCIDTIPKSRCTAFQCKHSMKYRLSFCRKTCGTC | ShK-A1/T2 | 17 |
| RSCADTIPKSRCTAFQCKHSMKYRLSFCRKTCGTC | ShK-A1/A4 | 18 |
| RSCADTIPKSRCTAAQCKHSMKYRLSFCRKTCGTC | ShK-A4/A15 | 19 |
| RSCADTIPKSRCTAAQCKHSMKYRASFCRKTCGTC | ShK-A4/A15/A25 | 20 |
| RSCIDAIPKSRCTAFQCKHSMKYRLSFCRKTCGTC | ShK-A6 | 21 |
| RSCIDTIPKSRCTAFQCKHSMKYRLSFCRKTCGTC-amide | ShK-T6 | 22 |
| RSCIDYIPKSRCTAFQCKHSMKYRLSFCRKTCGTC | ShK-Y6 | 23 |
| RSCIDLIPKSRCTAFQCKHSMKYRLSFCRKTCGTC | ShK-L6 | 24 |
| RSCIDTAPKSRCTAFQCKHSMKYRLSFCRKTCGTC | ShK-A7 | 25 |
| RSCADTIPKSRCTAFQCKHSMKYRLSFCRKTCGTC | ShK-A4 | 26 |
| RSCIDTIAKSRCTAFQCKHSMKYRLSFCRKTCGTC | ShK-A8 | 27 |
| RSCIDTIPASRCTAFQCKHSMKYRLSFCRKTCGTC | ShK-A9 | 28 |
| RSCIDTIPESRCTAFQCKHSMKYRLSFCRKTCGTC | ShK-E9 | 29 |
| RSCIDTIPQSRCTAFQCKHSMKYRLSFCRKTCGTC | ShK-Q9 | 30 |
| RSCIDTIPKARCTAFQCKHSMKYRLSFCRKTCGTC | ShK-A10 | 31 |
| RSCIDTIPKSACTAFQCKHSMKYRLSFCRKTCGTC | ShK-A11 | 32 |
| RSCIDTIPKSECTAFQCKHSMKYRLSFCRKTCGTC | ShK-E11 | 33 |
| RSCIDTIPKSQCTAFQCKHSMKYRLSFCRKTCGTC | ShK-Q11 | 34 |
| RSCIDTIPKSRCAAFQCKHSMKYRLSFCRKTCGTC | ShK-A13 | 35 |
| RSCIDTIPKSRCTAAQCKHSMKYRLSFCRKTCGTC | ShK-A15 | 36 |
| RSCIDTIPKSRCTAWQCKHSMKYRLSFCRKTCGTC | ShK-W15 | 37 |
| RSCIDTIPKSRCTA[X(s1)]QCKHSMKYRLSFCRKTCGTC | ShK-X15 | 38 |
| RSCIDTIPKSRCTAAQCKHSMKYRASFCRKTCGTC | ShK-Al5/A25 | 39 |
| RSCIDTIPKSRCTAFACKHSMKYRLSFCRKTCGTC | ShK-A16 | 40 |
| RSCIDTIPKSRCTAFECKHSMKYRLSFCRKTCGTC | ShK-E16 | 41 |
| RSCIDTIPKSRCTAFQCAHSMKYRLSFCRKTCGTC | ShK-A18 | 42 |
| RSCIDTIPKSRCTAFQCEHSMKYRLSFCRKTCGTC | ShK-E18 | 43 |
| RSCIDTIPKSRCTAFQCKASMKYRLSFCRKTCGTC | ShK-A19 | 44 |
| RSCIDTIPKSRCTAFQCKKSMKYRLSFCRKTCGTC | ShK-K19 | 45 |
| RSCIDTIPKSRCTAFQCKHAMKYRLSFCRKTCGTC | ShK-A20 | 46 |
| RSCIDTIPKSRCTAFQCKHSAKYRLSFCRKTCGTC | ShK-A21 | 47 |
| RSCIDTIPKSRCTAFQCKHS+X(s2)+KYRLSFCRKTCGTC | ShK-X21 | 48 |
| RSCIDTIPKSRCTAFQCKHS(Nle)KYRLSFCRKTCGTC | ShK-Nle21 | 49 |
| RSCIDTIPKSRCTAFQCKHSMAYRLSFCRKTCGTC | ShK-A22 | 50 |
| RSCIDTIPKSRCTAFQCKHSMEYRLSFCRKTCGTC | ShK-E22 | 51 |
| RSCIDTIPKSRCTAFQCKHSMRYRLSFCRKTCGTC | ShK-R22 | 52 |
| RSCIDTIPKSRCTAFQCKHSM[X(s3)]YRLSFCRKTCGTC | ShK-X22 | 53 |
| RSCIDTIPKSRCTAFQCKHSM(Nle)YRLSFCRKTCGTC | ShK-Nle22 | 54 |
| RSCIDTIPKSRCTAFQCKHSM(Orn)YRLSFCRKTCGTC | ShK-Orn22 | 55 |
| RSCIDTIPKSRCTAFQCKHSM(Homocit)YRLSFCRKTCGTC | ShK-Homocit22 | 56 |
| RSCIDTIPKSRCTAFQCKHSM(Dap)YRLSFCRKTCGTC | ShK-diamino-propionic22 | 57 |
| RSCIDTIPKSRCTAFQCKHSMKARLSFCRKTCGTC | ShK-A23 | 58 |
| RSCIDTIPKSRCTAFQCKHSMKSRLSFCRKTCGTC | ShK-S23 | 59 |
| RSCIDTIPKSRCTAFQCKHSMKFRLSFCRKTCGTC | ShK-F23 | 60 |
| RSCIDTIPKSRCTAFQCKHSMK[X(s4)]RLSFCRKTCGTC | ShK-X23 | 61 |
| RSCIDTIPKSRCTAFQCKHSMK(NitroF)RLSFCRKTCGTC | ShK-Nitrophe23 | 62 |
| RSCIDTIPKSRCTAFQCKHSMK(AminoF)RLSFCRKTCGTCC | ShK-Aminophe23 | 63 |
| RSCIDTIPKSRCTAFQCKHSMK(BenzylF)RLSFCRKTCGTC | ShK-Benzylphe23 | 64 |
| RSCIDTIPKSRCTAFQCKHSMKYALSFCRKTCGTC | ShK-A24 | 65 |
| RSCIDTIPKSRCTAFQCKHSMKYELSFCRKTCGTC | ShK-E24 | 66 |
| RSCIDTIPKSRCTAFQCKHSMKYRASFCRKTCGTC | ShK-A25 | 67 |
| RSCIDTIPKSRCTAFQCKHSMKYRLAFCRKTCGTC | ShK-A26 | 68 |
| RSCIDTIPKSRCTAFQCKHSMKYRLSACRKTCGTC | ShK-A27 | 69 |
| RSCIDTIPKSRCTAFQCKHSMKYRLS[X(s27)]CRKTCGTC | ShK-X27 | 70 |
| RSCIDTIPKSRCTAFQCKHSMKYRLSFCAKTCGTC | ShK-A29 | 71 |
| RSCIDTIPKSRCTAFQCKHSMKYRLSFCRATCGTC | ShK-A30 | 72 |
| RSCIDTIPKSRCTAFQCKHSMKYRLSFCRKACGTC | ShK-A31 | 73 |
| RSCIDTIPKSRCTAFQCKHSMKYRLSFCRKTCGAC | ShK-A34 | 74 |
| SCADTIPKSRCTAFQCKHSMKYRLSFCRKTCGTC | ShK-A4d1 | 75 |
| SCADTIPKSRCTAAQCKHSMKYRLSFCRKTCGTC | ShK-A4/A15d1 | 76 |
| SCADTIPKSRCTAAQCKHSMKYRASFCRKTCGTC | ShK-A4/A15/A25d1 | 77 |
| SCIDAIPKSRCTAFQCKHSMKYRLSFCRKTCGTC | ShK-A6d1 | 78 |
| SCIDTAPKSRCTAFQCKHSMKYRLSFCRKTCGTC | ShK-A7d1 | 79 |
| SCIDTIAKSRCTAFQCKHSMKYRLSFCRKTCGTC | ShK-A8d1 | 80 |
| SCIDTIPASRCTAFQCKHSMKYRLSFCRKTCGTC | ShK-A9d1 | 81 |
| SCIDTIPESRCTAFQCKHSMKYRLSFCRKTCGTC | ShK-E9d1 | 82 |
| SCIDTIPQSRCTAFQCKHSMKYRLSFCRKTCGTC | ShK-Q9d1 | 83 |
| SCIDTIPKARCTAFQCKHSMKYRLSFCRKTCGTC | ShK-A10d1 | 84 |
| SCIDTIPKSACTAFQCKHSMKYRLSFCRKTCGTC | ShK-A11d1 | 85 |
| SCIDTIPKSECTAFQCKHSMKYRLSFCRKTCGTC | ShK-E11d1 | 86 |
| SCIDTIPKSQCTAFQCKHSMKYRLSFCRKTCGTC | ShK-Q11d1 | 87 |
| SCIDTIPKSRCAAFQCKHSMKYRLSFCRKTCGTC | ShK-A13d1 | 88 |
| SCIDTIPKSRCTAAQCKHSMKYRLSFCRKTCGTC | ShK-A15d1 | 89 |
| SCIDTIPKSRCTAWQCKHSMKYRLSFCRKTCGTC | ShK-W15d1 | 90 |
| SCIDTIPKSRCTA[X(s15)]QCKHSMKYRLSFCRKTCGTC | ShK-X15d1 | 91 |
| SCIDTIPKSRCTAAQCKHSMKYRASFCRKTCGTC | ShK-A15/A25d1 | 92 |
| SCIDTIPKSRCTAFACKHSMKYRLSFCRKTCGTC | ShK-A16d1 | 93 |
| SCIDTIPKSRCTAFECKHSMKYRLSFCRKTCGTC | ShK-E16d1 | 94 |
| SCIDTIPKSRCTAFQCAHSMKYRLSFCRKTCGTC | ShK-A18d1 | 95 |
| SCIDTIPKSRCTAFQCEHSMKYRLSFCRKTCGTC | ShK-E18d1 | 96 |
| SCIDTIPKSRCTAFQCKASMKYRLSFCRKTCGTC | ShK-A19d1 | 97 |
| SCIDTIPKSRCTAFQCKKSMKYRLSFCRKTCGTC | ShK-K19d1 | 98 |
| SCIDTIPKSRCTAFQCKHAMKYRLSFCRKTCGTC | ShK-A20d1 | 99 |
| SCIDTIPKSRCTAFQCKHSAKYRLSFCRKTCGTC | ShK-A21d1 | 100 |
| SCIDTIPKSRCTAFQCKHS[X(s2)]KYRLSFCRKTCGTC | ShK-X21d1 | 101 |
| SCIDTIPKSRCTAFQCKHS(Nle)KYRLSFCRKTCGTC | ShK-Nle21d1 | 102 |
| SCIDTIPKSRCTAFQCKHSMAYRLSFCRKTCGTC | ShK-A22d1 | 103 |
| SCIDTIPKSRCTAFQCKHSMEYRLSFCRKTCGTC | ShK-E22d1 | 104 |
| SCIDTIPKSRCTAFQCKHSMRYRLSFCRKTCGTC | ShK-R22d1 | 105 |
| SCIDTIPKSRCTAFQCKHSM[X(s3)]YRLSFCRKTCGTC | ShK-X22d1 | 106 |
| SCIDTIPKSRCTAFQCKHSM(Nle)YRLSFCRKTCGTC | ShK-Nle22d1 | 107 |
| SCIDTIPKSRCTAFQCKHSM(Orn)YRLSFCRKTCGTC | ShK-Orn22d1 | 108 |
| SCIDTIPKSRCTAFQCKHSM(Homocit)YRLSFCRKTCGTC | ShK-Homocit22d1 | 109 |
| SCIDTIPKSRCTAFQCKHSM(Dap)YRLSFCRKTCGTC | ShK-Dap22d1 | 110 |
| SCIDTIPKSRCTAFQCKHSMKARLSFCRKTCGTC | ShK-A23d1 | 111 |
| SCIDTIPKSRCTAFQCKHSMKSRLSFCRKTCGTC | ShK-S23d1 | 112 |
| SCIDTIPKSRCTAFQCKHSMKFRLSFCRKTCGTC | ShK-F23d1 | 113 |
| SCIDTIPKSRCTAFQCKHSMK[X(s4)]RLSFCRKTCGTC | ShK-X23d1 | 114 |
| SCIDTIPKSRCTAFQCKHSMK(NitroF)RLSFCRKTCGTC | ShK-Nitrophe23d1 | 115 |
| SCIDTIPKSRCTAFQCKHSMK(AminoF)RLSFCRKTCGTC | ShK-Aminophe23d1 | 116 |
| SCIDTIPKSRCTAFQCKHSMK(BenzylF)RLSFCRKTCGT | ShK-Benzylphe23d1 | 117 |
| SCIDTIPKSRCTAFQCKHSMKYALSFCRKTCGTC | ShK-A24d1 | 118 |
| SCIDTIPKSRCTAFQCKHSMKYELSFCRKTCGTC | ShK-E24d1 | 119 |
| SCIDTIPKSRCTAFQCKHSMKYRASFCRKTCGTC | ShK-A25d1 | 120 |
| SCIDTIPKSRCTAFQCKHSMKYRLAFCRKTCGTC | ShK-A26d1 | 121 |
| SCIDTIPKSRCTAFQCKHSMKYRLSACRKTCGTC | ShK-A27d1 | 122 |
| SCIDTIPKSRCTAFQCKHSMKYRLS[X(s5)]CRKTCGTC | ShK-X27d1 | 123 |
| SCIDTIPKSRCTAFQCKHSMKYRLSFCAKTCGTC | ShK-A29d1 | 124 |
| SCIDTIPKSRCTAFQCKHSMKYRLSFCRATCGTC | ShK-A30d1 | 125 |
| SCIDTIPKSRCTAFQCKHSMKYRLSFCRKACGTC | ShK-A31d1 | 126 |
| SCIDTIPKSRCTAFQCKHSMKYRLSFCRKTCGAC | ShK-A34d1 | 127 |
| YSCIDTIPKSRCTAFQCKHSMKYRLSFCRKTCGTC | ShK-Y1 | 128 |
| KSCIDTIPKSRCTAFQCKHSMKYRLSFCRKTCGTC | ShK-K1 | 129 |
| HSCIDTIPKSRCTAFQCKHSMKYRLSFCRKTCGTC | ShK-H1 | 130 |
| QSCIDTIPKSRCTAFQCKHSMKYRLSFCRKTCGTC | ShK-Q1 | 131 |
| PPRSCIDTIPKSRCTAFQCKHSMKYRLSFCRKTCGTC | PP-ShK | 132 |
| MRSCIDTIPKSRCTAFQCKHSMKYRLSFCRKTCGTC | M-ShK | 133 |
| GRSCIDTIPKSRCTAFQCKHSMKYRLSFCRKTCGTC | G-ShK | 134 |
| YSCIDTIPKSRCTAFQCKHSMAYRLSFCRKTCGTC | ShK-Y1/A22 | 135 |
| KSCIDTIPKSRCTAFQCKHSMAYRLSFCRKTCGTC | ShK-K1/A22 | 136 |
| HSCIDTIPKSRCTAFQCKHSMAYRLSFCRKTCGTC | ShK-H1/A22 | 137 |
| QSCIDTIPKSRCTAFQCKHSMAYRLSFCRKTCGTC | ShK-Q1/A22 | 138 |
| PPRSCIDTIPKSRCTAFQCKHSMAYRLSFCRKTCGTC | PP-ShK-A22 | 139 |
| MRSCIDTIPKSRCTAFQCKHSMAYRLSFCRKTCGTC | M-ShK-A22 | 140 |
| GRSCIDTIPKSRCTAFQCKHSMAYRLSFCRKTCGTC | G-ShK-A22 | 141 |
| RSCIDTIPASRCTAFQCKHSMAYRLSFCRKTCGTC | ShK-A9/A22 | 142 |
| SCIDTIPASRCTAFQCKHSMAYRLSFCRKTCGTC | ShK-A9/A22d1 | 143 |
| RSCIDTIPVSRCTAFQCKHSMKYRLSFCRKTCGTC | ShK-V9 | 144 |
| RSCIDTIPVSRCTAFQCKHSMAYRLSFCRKTCGTC | ShK-V9/A22 | 145 |
| SCIDTIPVSRCTAFQCKHSMKYRLSFCRKTCGTC | ShK-V9d1 | 146 |
| SCIDTIPVSRCTAFQCKHSMAYRLSFCRKTCGTC | ShK-V9/A22d1 | 147 |
| RSCIDTIPESRCTAFQCKHSMAYRLSFCRKTCGTC | ShK-E9/A22 | 148 |
| SCIDTIPESRCTAFQCKHSMAYRLSFCRKTCGTC | ShK-E9/A22d1 | 149 |
| RSCIDTIPKSACTAFQCKHSMAYRLSFCRKTCGTC | ShK-A11/A22 | 150 |
| SCIDTIPKSACTAFQCKHSMAYRLSFCRKTCGTC | ShK-A11/A22d1 | 151 |
| RSCIDTIPKSECTAFQCKHSMAYRLSFCRKTCGTC | ShK-E11/A22 | 152 |
| SCIDTIPKSECTAFQCKHSMAYRLSFCRKTCGTC | ShK-E11/A22d1 | 153 |
| RSCIDTIPKSRCTDFQCKHSMKYRLSFCRKTCGTC | ShK-D14 | 154 |
| RSCIDTIPKSRCTDFQCKHSMAYRLSFCRKTCGTC | ShK-D14/A22 | 155 |
| SCIDTIPKSRCTDFQCKHSMKYRLSFCRKTCGTC | ShK-D14d1 | 156 |
| SCIDTIPKSRCTDFQCKHSMAYRLSFCRKTCGTC | ShK-D14/A22d1 | 157 |
| RSCIDTIPKSRCTAAQCKHSMAYRLSFCRKTCGTC | ShK-A15/A22 | 158 |
| SCIDTIPKSRCTAAQCKHSMAYRLSFCRKTCGTC | ShK-A15/A22d1 | 159 |
| RSCIDTIPKSRCTAIQCKHSMKYRLSFCRKTCGTC | ShK-I15 | 160 |
| RSCIDTIPKSRCTAIQCKHSMAYRLSFCRKTCGTC | ShK-I15/A22 | 161 |
| SCIDTIPKSRCTAIQCKHSMKYRLSFCRKTCGTC | ShK-I15d1 | 162 |
| SCIDTIPKSRCTAIQCKHSMAYRLSFCRKTCGTC | ShK-I15/A22d1 | 163 |
| RSCIDTIPKSRCTAVQCKHSMKYRLSFCRKTCGTC | ShK-V15 | 164 |
| RSCIDTIPKSRCTAVQCKHSMAYRLSFCRKTCGTC | ShK-V15/A22 | 165 |
| SCIDTIPKSRCTAVQCKHSMKYRLSFCRKTCGTC | ShK-V15d1 | 166 |
| SCIDTIPKSRCTAVQCKHSMAYRLSFCRKTCGTC | ShK-V15/A22d1 | 167 |
| RSCIDTIPKSRCTAFRCKHSMKYRLSFCRKTCGTC | ShK-R16 | 168 |
| RSCIDTIPKSRCTAFRCKHSMAYRLSFCRKTCGTC | ShK-R16/A22 | 169 |
| SCIDTIPKSRCTAFRCKHSMKYRLSFCRKTCGTC | ShK-R16d1 | 170 |
| SCIDTIPKSRCTAFRCKHSMAYRLSFCRKTCGTC | ShK-R16/A22d1 | 171 |
| RSCIDTIPKSRCTAFKCKHSMKYRLSFCRKTCGTC | ShK-K16 | 172 |
| RSCIDTIPKSRCTAFKCKHSMAYRLSFCRKTCGTC | ShK-K16/A22 | 173 |
| SCIDTIPKSRCTAFKCKHSMKYRLSFCRKTCGTC | ShK-K16d1 | 174 |
| SCIDTIPKSRCTAFKCKHSMAYRLSFCRKTCGTC | ShK-K16/A22d1 | 175 |
| RSCIDTIPASECTAFQCKHSMKYRLSFCRKTCGTC | ShK-A9/E11 | 176 |
| RSCIDTIPASECTAFQCKHSMAYRLSFCRKTCGTC | ShK-A9/E11/A22 | 177 |
| SCIDTIPASECTAFQCKHSMKYRLSFCRKTCGTC | ShK-A9/E11d1 | 178 |
| SCIDTIPASECTAFQCKHSMAYRLSFCRKTCGTC | ShK-A9/E11/A22d1 | 179 |
| RSCIDTIPVSECTAFQCKHSMKYRLSFCRKTCGTC | ShK-V9/E11 | 180 |
| RSCIDTIPVSECTAFQCKHSMAYRLSFCRKTCGTC | ShK-V9/E11/A22 | 181 |
| SCIDTIPVSECTAFQCKHSMKYRLSFCRKTCGTC | ShK-V9/E11d1 | 182 |
| SCIDTIPVSECTAFQCKHSMAYRLSFCRKTCGTC | ShK-V9/E11/A22d1 | 183 |
| RSCIDTIPVSACTAFQCKHSMKYRLSFCRKTCGTC | ShK-V9/A11 | 184 |
| RSCIDTIPVSACTAFQCKHSMAYRLSFCRKTCGTC | ShK-V9/A11/A22 | 185 |
| SCIDTIPVSACTAFQCKHSMKYRLSFCRKTCGTC | ShK-V9/A11d1 | 186 |
| SCIDTIPVSACTAFQCKHSMAYRLSFCRKTCGTC | ShK-V9/A11/A22d1 | 187 |
| RSCIDTIPASACTAFQCKHSMKYRLSFCRKTCGTC | ShK-A9/A11 | 188 |
| RSCIDTIPASACTAFQCKHSMAYRLSFCRKTCGTC | ShK-A9/A11/A22 | 189 |
| SCIDTIPASACTAFQCKHSMKYRLSFCRKTCGTC | ShK-A9/A11d1 | 190 |
| SCIDTIPASACTAFQCKHSMAYRLSFCRKTCGTC | ShK-A9/A11/A22d1 | 191 |
| RSCIDTIPKSECTDIRCKHSMKYRLSFCRKTCGTC | ShK-E11/D14/I15/R16 | 192 |
| RSCIDTIPKSECTDIRCKHSMAYRLSFCRKTCGTC | ShK-E11/D14/I15/R16/A22 | 193 |
| SCIDTIPKSECTDIRCKHSMKYRLSFCRKTCGTC | ShK-E11/D14/I15/R16d1 | 194 |
| SCIDTIPKSECTDIRCKHSMAYRLSFCRKTCGTC | ShK-E11/D14/I15/R16/A22d1 | 195 |
| RSCIDTIPVSECTDIRCKHSMKYRLSFCRKTCGTC | ShK-V9/E11/D14/I15/R16 | 196 |
| RSCIDTIPVSECTDIRCKHSMAYRLSFCRKTCGTC | ShK-V9/E11/D14/I15/R16/A22 | 197 |
| SCIDTIPVSECTDIRCKHSMKYRLSFCRKTCGTC | ShK-V9/E11/D14/I15/R16d1 | 198 |
| SCIDTIPVSECTDIRCKHSMAYRLSFCRKTCGTC | ShK-V9/E11/D14/I15/R16/A22d1 | 199 |
| RSCIDTIPVSECTDIQCKHSMKYRLSFCRKTCGTC | ShK-V9/E11/D14/I15 | 200 |
| RSCIDTIPVSECTDIQCKHSMAYRLSFCRKTCGTC | ShK-V9/E11/D14/I15/A22 | 201 |
| SCIDTIPVSECTDIQCKHSMKYRLSFCRKTCGTC | ShK-V9/E11/D14/I15d1 | 202 |
| SCIDTIPVSECTDIQCKHSMAYRLSFCRKTCGTC | ShK-V9/E11/D14/I15/A22d1 | 203 |
| RTCKDLIPVSECTDIRCKHSMKYRLSFCRKTCGTC | ShK-T2/K4/L6/V9/E11/D14/I15/R16 | 204 |
| RTCKDLIPVSECTDIRCKHSMAYRLSFCRKTCGTC | ShK-T2/K4/L6/V9/E11/D14/I15/R16/A22 | 205 |
| TCKDLIPVSECTDIRCKHSMKYRLSFCRKTCGTC | ShK-T2/K4/L6/V9/E11/D14/I15/R16d1 | 206 |
| TCKDLIPVSECTDIRCKHSMAYRLSFCRKTCGTC | ShK-T2/K4/L6/V9/E11/D14/I15/R16/A22d1 | 207 |
| (L-PhosphoTyr)-AEEAc- | ShK(L5) | 208 |
| RSCIDTIPKSRCTAFQCKHSMKYRLSFCRKTCGTC | | |
| (L-Tyr)-AEEAc- | ShK(L4) | 209 |
| RSCIDTIPKSRCTAFQCKHSMKYRLSFCRKTCGTC | | |
| (L-Tyr)-AEEAc- | ShK-198 | 210 |
| RSCIDTIPKSRCTAFQCKHSMKYRLSFCRKTCGTC-amide | | |
| QSCADTIPKSRCTAAQCKHSMKYRLSFCRKTCGTC | ShK-Q1/A4/A15 | 211 |
| QSCADTIPKSRCTAAQCKHSMAYRLSFCRKTCGTC | ShK-Q1/A4/A15/A22 | 212 |
| QSCADTIPKSRCTAAQCKHSM(Dap)YRLSFCRKTCGTC | ShK-Q1/A4/A15/Dap22 | 213 |
| QSCADTIPKSRCTAAQCKHSMKYRASFCRKTCGTC | ShK-Q1/A4/A15/A25 | 214 |
| QSCADTIPKSRCTAAQCKHSMAYRASFCRKTCGTC | ShK-Q1/A4/A15/A22/A25 | 215 |
| QSCADTIPKSRCTAAQCKHSM(Dap)YRASFCRKTCGTC | ShK-Q1/A4/A15/Dap22/A25 | 216 |
| (L-PhosphoTyr)-AEEAc- | ShK-186 | 217 |
| RSCIDTIPKSRCTAFQCKHSMKYRLSFCRKTCGTC-amide | | |
| (Para-phosphono-Phe)-AEEAc- | ShK-192 | 218 |
| RSCIDTIPKSRCTAFQCKHS(Nle)KYRLSFCRKTCGTC-amide | | |
| (Phosphonomethyl-Phe)-AEEAc- | ShK-191 | 219 |
| RSCIDTIPKSRCTAFQCKHSMKYRLSFCRKTCGTC-amide | | |
| (Phosphonomethyl-Phe)-AEEAc- | ShK-191/Nle21 | 220 |
| RSCIDTIPKSRCTAFQCKHS(Nle)KYRLSFCRKTCGTC-amide | | |
| DOTA-aminohexanoicacid-(L-Tyr)-AEEAc- | ShK-221 | 221 |
| RSCIDTIPKSRCTAFQCKHSMKYRLSFCRKTCGTC-amide | | |
| (Para-phosphono-Phe)-AEEAc- | ShK-223 | 222 |
| RSCIDTIPKSRCTAFKCKHS(Nle)KYRLSFCRKTCGTC-amide | | |
| (Para-phosphono-Phe)-AEEAc- | ShK-190 | 223 |
| RSCIDTIPKSRCTAFQCKHSMKYRLSFCRKTCGTC-amide | | |
| RSCIDTIPKSRCTAFQCKHS(Nle)(Dap)YRLSFCRKTCGTC | | 224 |
| (L-PhosphoTyr)-AEEAc- | | 257 |
| RSCIDTIPKSRCTAFQCKHS(Nle)KYRLSFCRKTCGTC | | |
| (L-Tyr)-AEEAc- | | 258 |
| RSCIDTIPKSRCTAFQCKHS(Nle)KYRLSFCRKTCGTC | | |
| (L-PhosphoTyr)-AEEAc- | | 259 |
| RSCIDTIPKSRCTAFQCKHS(Nle)KYRLSFCRKTCGTC-amide | | |
| (L-Tyr)-AEEAc- | | 260 |
| RSCIDTIPKSRCTAFQCKHS(Nle)KYRLSFCRKTCGTC-amide | | |

Notes:
X(s1), X(s2), X(s3), etc. each refer independently to nonfunctional amino acid residues.
N-acetylR refers to N-acetylarginine
Nle refers to Norleucine
Orn refers to Ornithine
Homocit refers to Homocitrulline
NitroF refers to Nitrophenylalanine
AminoF refers to Aminophenylalanine
BenzylF refers to Benzylphenylalanine
AEEAc refers to Aminoethyloxyethyloxyacetic acid
Dap refers to Diaminopropionic acid
DOTA refers to 1,4,7,10-tetraazacyclododecane-1,4,7,10-tetraacetic acid

Those skilled in the art are aware of techniques for designing toxin-based therapeutic proteins with enhanced properties, such as alanine scanning, rational design based on alignment mediated mutagenesis using known sequences, and/or molecular modeling. For example, toxin-based therapeutic proteins can be designed to remove protease cleavage sites (e.g., trypsin cleavage sites at K or R residues and/or chymotrypsin cleavage sites at F, Y, or W residues). Nonhydrolyzable phosphate substitutions also impart a stabilizing effect on the phosphate groups, as well as stability against phosphatase enzymes. Nonhydrolyzable phosphate groups include phosphonate analogs of phosphotyrosine such as 4-phosphonomethylphenylalanine (Pmp) 4-phosphonod ifluoromethylphenylalan ine (F2Pmp), paraphosphonophenylalanine, monofluorophosphonomethylphenylalanine, sulfono(difluormethyl)phenylalanine (F2Smp) and hydroxylphosphonomethylphenylalanine. In other embodiments, phosphotyrosine mimetics can be used such as OMT, FOMT, and other analogs that utilize carboxylic acid groups to replicate phosphate functionality as described in Burke and Lee, Acc. Chem. Res., 36, 426-433 (2003). In a further embodiment, nonhydrolyzable analogs include methyl-, aryloxy-, and thio-ethyl phosphonic acids. In a still further embodiment, nonhydrolyzable phosphate derivatives include difluoromethylenephosphonic and difluoromethylenesulfonic acid.

To improve the pharmacokinetic and pharmacodynamic (PK/PD) properties of the structure of toxin-based therapeutic proteins, residues that are sensitive to degradation properties can be substituted, replaced, or modified. Modification of the C-terminal acid function with an amide can also impart stability. These changes to the primary structure of toxin-based therapeutic proteins can be combined with an anionic moiety at the N-terminus to produce a stable and selective Kv1.3 blocker. In order to produce a toxin-based therapeutic protein with a higher half-life in vivo, variants or modifications of the proteins can be prepared wherein key proteolytic digestion sites may be substituted to reduce protease susceptibility. This may include substitution of nonessential residues with conservative isosteric replacements (e.g., Lys to Lys (acetyl) or Gln) and or neutral replacements (Ala).

“Variants” of toxin-based therapeutic proteins disclosed herein include proteins having one or more amino acid additions, deletions, stop positions, or substitutions, as compared to a toxin-based or ShK-based protein disclosed herein.

An amino acid substitution can be a conservative or a non-conservative substitution. Variants of toxin-based therapeutic proteins disclosed herein can include those having one or more conservative amino acid substitutions. A “conservative substitution” involves a substitution found in one of the following conservative substitutions groups: Group 1: Alanine (Ala; A), Glycine (Gly; G), Serine (Ser; S), Threonine (Thr; T); Group 2: Aspartic acid (Asp; D), Glutamic acid (Glu; E); Group 3: Asparagine (Asn; N), Glutamine (Gln; Q); Group 4: Arginine (Arg; R), Lysine (Lys; K), Histidine (His; H); Group 5: Isoleucine (Ile; I), Leucine (Leu; L), Methionine (Met; M), Valine (Val; V); and Group 6: Phenylalanine (Phe; F), Tyrosine (Tyr; Y), Tryptophan (Trp; W).

Additionally, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other groups including amino acids that are considered conservative substitutions for one another include: sulfur-containing: Met and Cys; acidic: Asp, Glu, Asn, and Gin; small aliphatic, nonpolar or slightly polar residues: Ala, Ser, Thr, Pro, and Gly; polar, negatively charged residues and their amides: Asp, Asn, Glu, and Gin; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company.

Variants of toxin-based therapeutic proteins disclosed herein also include proteins with at least 70% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to a protein sequence disclosed herein.

Variants of toxin-based therapeutic proteins for use in the depot formulations disclosed herein based on toxin-based proteins include proteins that share: 70% sequence identity with any of SEQ ID NO:225-256; 75% sequence identity with any of SEQ ID NO:225-256; 80% sequence identity with any of SEQ ID NO:225-256; 81% sequence identity with any of SEQ ID NO:225-256; 82% sequence identity with any of SEQ ID NO:225-256; 83% sequence identity with any of SEQ ID NO:225-256; 84% sequence identity with any of SEQ ID NO:225-256; 85% sequence identity with any of SEQ ID NO:225-256; 86% sequence identity with any of SEQ ID NO: 225-256; 87% sequence identity with any of SEQ ID NO:225-256; 88% sequence identity with any of SEQ ID NO:225-256; 89% sequence identity with any of SEQ ID NO:225-256; 90% sequence identity with any of SEQ ID NO:225-256; 91% sequence identity with any of SEQ ID NO:225-256; 92% sequence identity with any of SEQ ID NO:225-256; 93% sequence identity with any of SEQ ID NO:225-256; 94% sequence identity with any of SEQ ID NO:225-256; 95% sequence identity with any of SEQ ID NO:225-256; 96% sequence identity with any of SEQ ID NO:225-256; 97% sequence identity with any of SEQ ID NO:225-256; 98% sequence identity with any of SEQ ID NO:225-256; or 99% sequence identity with any of SEQ ID NO:225-256.

Variants of toxin-based therapeutic proteins for use in the depot formulations disclosed herein based on ShK-based proteins include proteins that share: 80% sequence identity with any of SEQ ID NO:1-224 and/or SEQ ID NO:257-260; 81% sequence identity with any of SEQ ID NO:1-224 and/or SEQ ID NO:257-260; 82% sequence identity with any of SEQ ID NO:1-224 and/or SEQ ID NO:257-260; 83% sequence identity with any of SEQ ID NO:1-224 and/or SEQ ID NO:257-260; 84% sequence identity with any of SEQ ID NO:1-224 and/or SEQ ID NO:257-260; 85% sequence identity with any of SEQ ID NO:1-224 and/or SEQ ID NO:257-260; 86% sequence identity with any of SEQ ID NO:1-224 and/or SEQ ID NO:257-260; 87% sequence identity with any of SEQ ID NO:1-224 and/or SEQ ID NO:257-260; 88% sequence identity with any of SEQ ID NO:1-224 and/or SEQ ID NO:257-260; 89% sequence identity with any of SEQ ID NO:1-224 and/or SEQ ID NO:257-260; 90% sequence identity with any of SEQ ID NO:1-224 and/or SEQ ID NO:257-260; 91% sequence identity with any of SEQ ID NO:1-224 and/or SEQ ID NO:257-260; 92% sequence identity with any of SEQ ID NO:1-224 and/or SEQ ID NO:257-260; 93% sequence identity with any of SEQ ID NO:1-224 and/or SEQ ID NO:257-260; 94% sequence identity with any of SEQ ID NO:1-224 and/or SEQ ID NO:257-260; 95% sequence identity with any of SEQ ID NO:1-224 and/or SEQ ID NO:257-260; 96% sequence identity with any of SEQ ID NO:1-224 and/or SEQ ID NO:257-260; 97% sequence identity with any of SEQ ID NO:1-224 and/or SEQ ID NO:257-260; 98% sequence identity with any of SEQ ID NO:1-224 and/or SEQ ID NO:257-260; or 99% sequence identity with any of SEQ ID NO:1-224 and/or SEQ ID NO:257-260.

Particular exemplary embodiments include toxin-based therapeutic proteins wherein the proteins share 80% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity with SEQ ID NO:208. In another embodiment, variants include proteins sharing 80% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity with SEQ ID NO:209. In another embodiment, variants include proteins sharing 80% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity with SEQ ID NO:217. In another embodiment, variants include proteins sharing 80% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity, with SEQ ID NO:210. In another embodiment, variants include proteins sharing 80% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity with SEQ ID NO:218. In another embodiment, variants include proteins sharing 80% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity with SEQ ID NO:208. In another embodiment, variants include proteins sharing 80% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity with SEQ ID NO:257.

“% sequence identity” refers to a relationship between two or more sequences, as determined by comparing the sequences. In the art, “identity” also means the degree of sequence relatedness between protein sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine sequence identity are designed to give the best match between the sequences tested. Methods to determine sequence identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” mean any set of values or parameters which originally load with the software when first initialized.

“D-substituted analogs” include toxin-based therapeutic proteins disclosed herein having one more L-amino acids substituted with D-amino acids. The D-amino acid can be the same amino acid type as that found in the protein sequence or can be a different amino acid. Accordingly, D-analogs are also variants.

“Modifications” include toxin-based therapeutic proteins disclosed herein, wherein one or more amino acids have been replaced with a non-amino acid component, or where the amino acid has been conjugated to a functional group or a functional group has been otherwise associated with an amino acid or protein. The modified amino acid may be, e.g., a glycosylated amino acid, a PEGylated amino acid, a farnesylated amino acid, an acetylated amino acid, a biotinylated amino acid, an amino acid conjugated to a lipid moiety, an amino acid conjugated to human serum albumin, or an amino acid conjugated to an organic derivatizing agent. The presence of modified amino acids may be advantageous in, for example, (a) increasing protein serum half-life and/or functional in vivo half-life, (b) reducing protein antigenicity, (c) increasing protein storage stability, (d) increasing protein solubility, (e) prolonging circulating time, and/or (f) increasing bioavailability, e.g. increasing the area under the curve (AUCsc). Amino acid(s) can be modified, for example, co-translationally or post-translationally during recombinant production (e.g., N-linked glycosylation at N-X-S/T motifs during expression in mammalian cells) or modified by synthetic means. The modified amino acid can be within the sequence or at the terminal end of a sequence. Modifications can include derivatives as described elsewhere herein.

The C-terminus may be a carboxylic acid or an amide group, preferably a carboxylic acid group for each of the toxin-based therapeutic proteins. The present disclosure also relates to the toxin-based therapeutic proteins further modified by (i) additions made to the C-terminus, such as Tyr, iodo-Tyr, a fluorescent tag, or (ii) additions made to the N-terminus, such as Tyr, iodo-Tyr, pyroglutamate, or a fluorescent tag.

In addition, residues or groups of residues known to the skilled artisan to improve stability can be added to the C-terminus and/or N-terminus. Also, residues or groups of residues known to the skilled artisan to improve oral availability can be added to the C-terminus and/or N-terminus.

In particular embodiments, the C-terminus is an acid (for example, COOH) or an amide (for example, CONH₂). “Amide” refers to NH₂, in particular embodiments, attached to the C-terminal end of a protein. In various embodiments, the C-terminal hydroxyl group (OH) of an acid is substituted with an amide. Such substitution is designated herein using the term “amide” or as the C-terminal amino acid-NH₂, as in “-Cys-NH₂.”

The safety, potency, and specificity of a variety of therapeutic proteins have been investigated, and attaching the protein to an organic or inorganic chemical entity that has an anionic charge has been shown to improve the suitability for use in pharmaceutical compositions. The site of attachment can be the N-terminus, but modifications are not limited to attachment at this site.

Examples of appropriate chemical entities include L-Pmp(OH₂); D-Pmp(OH₂); D-Pmp(OHEt); Pmp(Et2); D-Pmp(Et2); L-Tyr; L-Tyr(PO₃H₂) (p-phospho-Tyrosine); L-Phe(p-NH₂); L-Phe(p-CO₂H); L-Aspartate; D-Aspartate; L-Glutamate; and D-Glutamate. The abbreviations used are defined as follows: Pmp (p-phosphonomethyl-phenylalanine); and Ppa (p-phosphatityl-phenylalanine). Alternatives to PmP and Ppa include Pfp (p-Phosphono(difluoro-methyl)-Phenylalanine) and Pkp (p-Phosphono-methylketo-Phenylalanine).

Exemplary chemical entities can be attached by way of a linker, such as an aminoethyloxyethyloxy-acetyl acid linker (referred to herein as AEEAc), or by any other suitable means. Examples of chemical entity/linker combinations include AEEAc-L-Pmp(OH₂); AEEAc-D-Pmp(OH₂); AEEAc-D-Pmp(OHEt); AEEAc-L-Pmp(Et2); AEEAc-D-Pmp(Et2); AEEAc-L-Tyr; AEEAc-L-Tyr(PO₃H₂); AEEAc-L-Phe(p-NH₂); AEEAc-L-Phe(p-CO₂H); AEEAc-L-Aspartate; AEEAc-D-Aspartate; AEEAc-L-Glutamate; and AEEAc-D-Glutamate. In the chemical entities generally, where the amino acid residue has a chiral center, the D and/or L enantiomer of the amino acid residue can be used.

All toxin-based therapeutic proteins disclosed herein can be modified by the N-terminal attachment of AEEAc and/or an amide attachment at the C-terminal (for example, ShK-186 (SEQ ID NO: 217) and ShK-192 (SEQ ID NO: 218)). AEEAc can interchangeably refer to aminoethyloxyethyloxyacetic acid and Fmoc-aminoethyloxyethyloxyacetic acid when being used to describe the linker during the formation process. When being used to refer to the linker in specific proteins in their final state, the term refers to aminoethyloxyethyloxyacetic acid.

All toxin-based therapeutic proteins disclosed herein can be modified by the addition of polyethylene glycol (PEG), human serum albumin, antibodies, fatty acids, antibody fragments including the Fab and Fc regions, hydroxyethyl starch, dextran, oligosaccharides, polysialic acids, hyaluronic acid, dextrin, poly(2-ethyl 2-oxazolone), polyglutamic acid (PGA), N-(2-hydroxypropyl)methacrylamide copolymer (HPMA), unstructured hydrophilic sequences of amino acids including in particular the amino acids Ala, Glu, Gly, Ser, and Thr, and many other linkers and additions as described in Schmidt, S. R. (ed), Fusion Protein Targeting for Biopharmaceuticals: Applications and Challenges, John Wiley and Sons: Hoboken N.J., 2013. PEG groups can be attached to c amino groups of lysine using: (a) PEG succinimidyl carbonate, (b) PEG benzotriazole carbonate, (c) PEG dichlorotriazine, (d) PEG tresylate, (e) PEG p-nitrophenyl carbonate, (f) PEG trichlorophenyl carbonate, (g) PEG carbonylimidazole, and (h) PEG succinimidyl succinate. PEG groups can be attached to cysteines by degradable linkers including para- or ortho-disulfide of benzyl urethane. Site specific introduction of PEG can be achieved by reductive alkylation with PEG-aldehyde or by glyceraldehyde modification of alpha-amino groups in the presence of sodium cyanoborohydride. PEGylation chemistries have been described in numerous publications including Robert, et al., Advanced Drug Delivery Reviews, 54, 459-476 (2002). Oligosaccharides can be N-linked or O-linked. N-linked oligosaccharides, including polysialic acid are added by the producing cell line by attachment to the consensus sequence of Asn-Xxx-Ser/Thr where Xxx is anything but proline. O-linked oligosaccharides are attached to Ser or Thr.

Particular embodiments include toxin-based therapeutic proteins of SEQ ID NO: 1-260 to which an organic or inorganic chemical entity that has an anionic charge is attached via AEEAc.

Another example of a toxin-based therapeutic protein is an ShK-based DOTA-conjugate of ShK-186 (referred to as ShK-221). “DOTA” refers to 1,4,7,10-tetraazacyclododecane-1,4,7,10-tetraacetic acid which can be attached to the N-terminus of the therapeutic proteins disclosed herein via aminohexanoic acid. DOTA conjugation provides a site for chelating metal atoms such as Indium or Gadolinium. Other molecules that can be conjugated to therapeutic proteins disclosed herein include diethylene triamine pentaacetic acid (DTPA), Nitrilotriacetic acid (NTA), Ethylenediaminetetraacetic acid (EDTA), Iminodiacetic acid (IDA), ethylene glycol tetraacetic acid (EGTA), 1,2-bis(o-aminophenoxy)ethane-N,N,N′,N′-tetraacetic acid (BAPTA), 1,4,7-triazacyclononane-N,N′,N″-triacetic acid (NOTA), and related molecules.

The present disclosure is further directed to derivatives of the disclosed toxin-based therapeutic proteins. “Derivatives” include toxin-based therapeutic proteins having acylic permutations in which the cyclic permutants retain the native bridging pattern of the native protein. In one embodiment, the cyclized toxin-based therapeutic protein includes a linear toxin-based therapeutic protein and a protein linker, wherein the N- and C-termini of the linear toxin-based therapeutic protein are linked via the protein linker to form the amide cyclized protein backbone. In some embodiments, the protein linker includes amino acids selected from Gly, Ala, and combinations thereof.

Various cyclization methods can be applied to the toxin-based therapeutic proteins described herein. The toxin-based therapeutic proteins described herein can be readily cyclized using BOC-chemistry to introduce Ala, Gly, or Ala/Gly bridges, as well as combinations thereof or other residues as described by Schnolzer, et al., Int J Pept Protein Res., 40, 180-193 (1992). Cyclizing toxin-based therapeutic proteins can improve their stability, oral bioavailability, and reduce the susceptibility to proteolysis, without affecting the affinity of the toxin-based therapeutic proteins for their specific targets.

Each toxin-based therapeutic protein disclosed herein may also include additions, deletions, stop positions, substitutions, replacements, conjugations, associations, or permutations at any position including positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of a toxin-based therapeutic protein sequence disclosed herein. Accordingly, in particular embodiments each amino acid position of each toxin-based therapeutic protein can be an Xaa position wherein Xaa denotes an addition, deletion, stop position, substitution, replacement, conjugation, association, or permutation of the amino acid at the particular position. In particular embodiments, each toxin-based therapeutic protein has 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Xaa positions at one or more of positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60.

A toxin-based therapeutic protein can have more than one change (addition, deletion, stop position, substitution, replacement, conjugation, association, or permutation) and qualify as one or more of a variant, D-substituted analog, carboxy-terminal amide, modification, and/or derivative. That is, inclusion of one classification of variant, D-substituted analog, carboxy-terminal amide, modification, and/or derivative is not exclusive to inclusion in other classifications and all are collectively referred to as “toxin-based therapeutic proteins” herein. One example includes SEQ ID NO: 1 wherein the amino acid at position 21 is Nle and/or the amino acid at position 22 is replaced with diaminopropionic acid.

In any of the proteins where position 21 is a Met, the Met can be substituted to impart a stabilizing effect against oxidation. In one embodiment, a Met at position 21 is substituted with Nle. In any of SEQ ID NO: 1-256, having a Met at position 21, this Met can be substituted with Nle. In any of SEQ ID NO: 1-256, having a Lys at position 22, this Lys can be substituted with diaminopropionic acid. Accordingly, one embodiment disclosed herein includes SEQ ID NO: 1 wherein the Met at position 21 is substituted with Nle, an amide is present at the C-terminus and/or an anionic moiety is present at the N-terminus.

“Nonfunctional amino acid residue” refers to amino acid residues in D- or L-form having sidechains that lack acidic, basic, or aromatic groups. Exemplary nonfunctional amino acid residues include Meg, Gly, Ala, Val, Ile, Leu, and Nle.

In particular embodiments disclosed herein, the therapeutic protein has at least 20 amino acids, at least 21 amino acids, at least 22 amino acids, at least 23 amino acids, at least 24 amino acids, at least 25 amino acids, at least 26 amino acids, at least 27 amino acids, at least 28 amino acids, at least 29 amino acids, at least 30 amino acids, at least 31 amino acids, at least 32 amino acids, at least 33 amino acids, at least 34 amino acids, at least 35 amino acids, at least 36 amino acids, at least 37 amino acids, at least 38 amino acids, at least 39 amino acids, at least 40 amino acids, at least 41 amino acids, at least 42 amino acids, at least 43 amino acids, at least 44 amino acids, at least 45 amino acids, at least 46 amino acids, at least 47 amino acids, at least 48 amino acids, at least 49 amino acids, at least 50 amino acids, at least 51 amino acids, at least 52 amino acids, at least 53 amino acids, at least 54 amino acids, at least 55 amino acids, at least 56 amino acids, at least 57 amino acids, at least 58 amino acids, at least 59 amino acids, at least 60 amino acids, at least 61 amino acids, at least 62 amino acids, at least 63 amino acids, at least 64 amino acids, at least 65 amino acids, at least 66 amino acids, at least 67 amino acids, at least 68 amino acids, at least 69 amino acids, at least 70 amino acids, at least 71 amino acids, at least 72 amino acids, at least 73 amino acids, at least 74 amino acids, at least 75 amino acids, at least 76 amino acids, at least 77 amino acids, at least 78 amino acids, at least 79 amino acids, or at least 80 amino acids.

In additional embodiments, the therapeutic protein has 20 amino acids, 21 amino acids, 22 amino acids, 23 amino acids, 24 amino acids, 25 amino acids, 26 amino acids, 27 amino acids, 28 amino acids, 29 amino acids, 30 amino acids, 31 amino acids, 32 amino acids, 33 amino acids, 34 amino acids, 35 amino acids, 36 amino acids, 37 amino acids, 38 amino acids, 39 amino acids, 40 amino acids, 41 amino acids, 42 amino acids, 43 amino acids, 44 amino acids, 45 amino acids, 46 amino acids, 47 amino acids, 48 amino acids, 49 amino acids, 50 amino acids, 51 amino acids, 52 amino acids, 53 amino acids, 54 amino acids, 55 amino acids, 56 amino acids, 57 amino acids, 58 amino acids, 59 amino acids, 60 amino acids, 61 amino acids, 62 amino acids, 63 amino acids, 64 amino acids, 65 amino acids, 66 amino acids, 67 amino acids, 68 amino acids, 69 amino acids, 70 amino acids, 71 amino acids, 72 amino acids, 73 amino acids, 74 amino acids, 75 amino acids, 76 amino acids, 77 amino acids, 78 amino acids, 79 amino acids, or 80 amino acids.

In additional embodiments disclosed herein the therapeutic protein has at least one disulfide bridge, at least two disulfide bridges, at least three disulfide bridges, at least four disulfide bridges, or at least five disulfide bridges.

In additional embodiments, the therapeutic protein has one disulfide bridge, two disulfide bridges, three disulfide bridges, four disulfide bridges, or five disulfide bridges.

Therapeutic proteins also suitable for use in the depot formulations disclosed herein include those having a molecular weight between 500 and 50,000 Daltons.

Particularly relevant therapeutic proteins include those that act upon cation channels such as Na⁺, K⁺, or Ca²⁺ channels, anion channels such as Cl⁻ channels or ligand-gated channels such as nicotinic acetyl choline receptors (NAChRs). These channels include both ligand and voltage-gated ion channels that are present extracellularly and/or intracellularly. Extracellular channels or receptors include kanate; α-amino-3-hydroxy-5-methyl-4-isoxazole propionic acid (AMPA); N-methyl-D-aspartate (NMDA) and acetylcholine receptors (such as α9/α10 subtype (nAChR)); serotonin (5-hydroxytryptamine, 5-HT) receptors; and glycine and γ-butyric (GABA) receptors. Intracellular receptors can include cyclic AMP (cAMP), cyclic GMP (cGMP), Ca²⁺, and G-protein receptors.

Particular examples of therapeutic proteins useful with the depot formulations disclosed herein include toxin proteins, including ShK proteins, that target voltage gated channels. Exemplary voltage gated channels include Kv1.1, Kv1.2, Kv1.3, Kv1.4, Kv1.5, Kv1.6, Kv1.7, Kv2.1, Kv3.1, Kv3.2, Kv11.1, Kc1.1, Kc2.1, Kc3.1, Nav1.2, Nav1.4, and Cav1.2 channels.

Prodrugs of the therapeutic proteins described herein can also be used. The term “prodrug” refers to a therapeutic protein that can undergo biotransformation (e.g., either spontaneous or enzymatic) within a subject to release, or to convert (e.g., enzymatically, mechanically, electromagnetically, etc.) an active or more active form of the protein. Prodrugs can be used to overcome issues associated with stability, toxicity, lack of specificity, or limited bioavailability. Exemplary prodrugs include an active protein and a chemical masking group (e.g., a group that reversibly suppresses the activity of the protein). Some preferred prodrugs are variants or modifications of proteins that have sequences that are cleavable under metabolic conditions. Exemplary prodrugs become active or more active in vivo or in vitro when they undergo a biochemical transformation (e.g., phosphorylation, hydrogenation, dehydrogenation, glycosylation, etc.). Prodrugs often offer advantages of solubility, tissue compatibility, or delayed release (See e.g., Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, Calif. (1992)).

Because an initial burst typically occurs with water soluble species of the above mentioned therapeutic proteins when administered in water-based vehicles, it is advantageous to use the PLG-based depot formulations disclosed herein with such water soluble species. The octanol-water partition coefficient (Kow) for such a protein is 1 or less, but more than 0.1. The protein may be formulated in a salt form, especially when the molecule has a basic group such as an amino residue. Salt forms may be adducts of acids such as hydrochloric acid, sulfuric acid, nitric acid, and organic acids such as carbonic and succinic acid.

Therapeutic proteins used with the depot formulations disclosed herein can also be molecularly engineered to show robust acid stability. Specifically, C-terminal amidation, a non-oxidable Nle substitution, and/or non-hydrolyzable L-phosphotyrosine substitution at the N-terminus can be performed to adapt the therapeutic protein to the acidic microenvironment and physiological environment it would be subject to in a depot formulation.

The proper amount of a therapeutic protein depends on the nature of the protein, but usually falls into the range of 0.001% to 90% w/w, based upon the composition of the biodegradable polymer used in the depot formulation. Additional embodiments include, in w/w, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10%.

METHODS OF FORMING DEPOT FORMULATIONS. Regarding methods of producing examples of depot formulations disclosed herein, any known microencapsulation procedures for entrapping the therapeutic protein can be employed, including drying-in-water methods, spray drying methods, coacervation methods, or equivalents thereof.

In particular embodiments, aqueous soluble or dispersable therapeutic proteins can be combined with excipients such as salts, buffers, polyols, sugars, amino acids, surfactants, stabilizers, and release modifiers and mixed with polymers and solvents, creating a multiphase system that can be mechanically converted to a microemulsion through homogenization, spray-drying, coacervacion, ultrasonication, and/or microfluidization. This primary water/oil (w/o) emulsion can then be added to an aqueous continuous phase including stabilizers such as surfactants and buffers and further dispersed to form a water in oil in water (w/o/w) emulsion with polymer particles that harden or ripen over time through loss of solvent evaporation and stirring/mixing.

The aqueous suspension can be further concentrated by using methods such as centrifugal separation to enrich a higher density phase (predominantly polymer particles), followed by removal of the upper (clear or slightly hazy) solution layer to the appropriate final volume to achieve the concentration desired. This approach can also be used to make the external, aqueous phase the desired pH, osmolarity, ionic strength, and buffer composition. In a similar manner, the suspension could be further diluted into aqueous compositions including saline, phosphate-buffered saline, sugar solutions, salts, buffers, and other excipients to achieve a desirable concentration (of weight percent solids, pH, osmolarity, or total drug dose) or diluted into excipients for a freeze-drying (lyophilization) step or additional processing.

As further described in the Examples below, the therapeutic protein can be dissolved in water to a final desired concentration and with buffer salts and excipients including surfactants. The complete w/o emulsion can be poured into a media bottle; the bottle charged with 20 mL of 0.5% DSS, 20 mM Phosphate pH 7.0, 0.05% PVA water solution. The mixture can be homogenized 5 minutes with a shear setting (26,000 revolutions per minute (rpm)), with the bottle in thermal contact with melting ice (0° C.). The generator probe can be kept immersed to limit frothing and spillage of liquid. The emulsion will turn a milky white color (similar to 1% milk). The pH can be tested and adjusted as needed into the range 5<pH<7.5. The formulation can be removed from shear and loosely capped to slow down evaporation, and stirred overnight at room temperature in a fume hood to allow solvent evaporation (dichloromethane). The formulation can then be filtered through a screen. The emulsion can be stored at room temperature with gentle end over end mixing to avoid settling/clumping. Alternatively, the solutions can be stored at 4° C. or lyophilized for longer shelf stability. Numerous appropriate lyophilization techniques are known to those of ordinary skill in the art.

METHODS OF USE. Methods disclosed herein include treating subjects (humans, veterinary animals (dogs, cats, reptiles, birds, etc.), livestock (horses, cattle, goats, pigs, chickens, etc.), and research animals (monkeys, rats, mice, fish, etc.)) with depot formulations disclosed herein to achieve sustained release of therapeutic proteins, salts or prodrugs thereof. The sustained release can deliver therapeutically effective amounts of the therapeutic proteins, salts or prodrugs thereof to the subject. Therapeutically effective amounts include those that provide effective amounts or effective levels (defined previously).

An “effective amount” is the amount of a therapeutic protein necessary to result in a desired physiological change in the subject. Effective amounts are often administered for research purposes. Effective amounts disclosed herein reduce, control, or eliminate the presence or activity of disorders of the immune system and/or reduce, control, or eliminate unwanted side effects of disorders of the immune system.

A “prophylactic treatment” includes a treatment administered to a subject who does not display signs or symptoms of a disorder of the immune system or displays only early signs or symptoms of the disorder of the immune system such that treatment is administered for the purpose of diminishing, preventing, or decreasing the risk of developing the disorder of the immune system further. Thus, a prophylactic treatment functions as a preventative treatment against a disorder of the immune system.

A “therapeutic treatment” includes a treatment administered to a subject who displays symptoms or signs of a disorder of the immune system and is administered to the subject for the purpose of diminishing or eliminating those signs or symptoms of the disorder of the immune system. The therapeutic treatment can reduce, control, or eliminate the presence or activity of disorders of the immune system, and/or reduce, control, or eliminate side effects of disorders of the immune system.

In particular embodiments, the therapeutic proteins disclosed herein are formulated in depot formulations for the therapeutic treatment of disorders or conditions of the immune system, including autoimmune diseases. In certain embodiments, the autoimmune disease or condition is psoriasis, psoriatic arthritis, multiple sclerosis, IPEX, systemic lupus erythematosus, lupus nephritis, type I diabetes, type II diabetes, Addison's disease, Celiac disease, dermatomyositis, Graves' disease, Hashimoto's thyroiditis, Myasthenia gravis, Pernicious anemia, rheumatoid arthritis, granulomatosis with polyangiitis (Wegener's disease), anti-neutrophil cytoplasmic autoantibody (ANCA) vasculitis, inflammatory bowel diseases, Alzheimer's disease, allergies, asthma, atopic dermatitis, graft-vs-host disease, tissue or organ transplantation, cardiovascular disease, vasculititis, small vessel vasculititis, giant cell arteritis, uveitis, Behcet's syndrome, non-alcoholic fatty liver disease including NASH, autoimmune liver disease, or Sjogren syndrome.

In one exemplary embodiment, the disease of the immune system is psoriasis. The impact of treatment can be evaluated using parameters including plaque body surface area involvement (% BSA), Psoriasis Area and Severity Index (PASI) components, and Investigator's global assessment of psoriasis (IGA, 5 point scale) patient global assessment of psoriasis, dermatology life quality index (DLQI), and psoriasis disability index (PDI). The impact of treatment on psoriatic plaques can be determined by evaluation of biopsies taken at 15, 30, or more days post injection using approaches including: plaque histopathology by H&E staining and evaluation by a pathologist; gene expression by qPCR for proinflammatory cytokines including IFNγ, TNFα, iNOS, IL-4, 8, 10, 17A, 17F, 17A/F, 20, 21,22, 23, CCL20, psoriasin, K16, and other cytokines; immunohistochemical characterization for cell activation/populations (KRT16 and Ki67); and/or measurement of mononuclear cell infiltration (CD3, HLA-DR, CD11c⁺, CD68, CD163, Kv1.3).

In a further embodiment, the effect on the systemic autoimmune/inflammation status during disease can be evaluated by parameters measured using techniques known in the art, including: measurement of plasma/serum biomarkers including IL-17A, IL-17F, IL-17A/F and other cytokines/chemokines; gene expression in whole blood total RNA; and/or analysis of peripheral blood mononuclear cell populations (CD4+ T cells: naïve, central memory, or effector memory T cells; CD8+ T cells: naïve, central memory, or effector memory T cells; regulatory T cells).

For administration, “therapeutically effective amounts” (also referred to herein as doses) can be initially estimated based on results from in vitro assays and/or animal model studies. Such information can be used to more accurately determine useful doses in subjects of interest.

The amount and concentration of a therapeutic protein in a depot formulation, as well as the quantity of the depot formulation administered to a subject, can be selected by a physician, veterinarian, or researcher based on clinically relevant factors, the solubility of a therapeutic protein in the depot formulation, the potency and activity of a therapeutic protein, and the manner of administration of the depot formulation. A depot formulation including a therapeutically effective amount of a therapeutic protein disclosed herein, or a pharmaceutically acceptable salt or prodrug thereof, can be administered to a subject for treatment of autoimmune diseases in a clinically safe and effective manner, including one or more separate administrations of the depot formulation. The amount per administered dose and the total amount administered can also depend on physical, physiological and psychological factors of the subject including target, body weight, severity of condition, type of autoimmune disease, previous or concurrent therapeutic interventions, idiopathy of the subject, and route of administration, among other considerations.

Useful doses can often range from 0.1 to 40,000 μg/kg or from 0.5 to 1 μg/kg. In other examples, useful doses can often range from 0.1 to 1 μg/kg, from 0.1 to 10 μg/kg, from 0.1 to 100 μg/kg, from 0.1 to 1,000 μg/kg, from 0.1 to 10,000 μg/kg, from 0.1 to 20,000 μg/kg, from 1 to 10 μg/kg, from 1 to 100 μg/kg, from 1 to 1,000 μg/kg, from 1 to 10,000 μg/kg, from 1 to 20,000 μg/kg, from 1 to 30,000 μg/kg, from 10 to 100 μg/kg, from 10 to 1,000 μg/kg, from 10 to 10,000 μg/kg, from 10 to 20,000 μg/kg, from 10 to 30,000 μg/kg, from 100 to 1,000 μg/kg, from 100 to 10,000 μg/kg, from 100 to 20,000 μg/kg, from 100 to 30,000 μg/kg, from 1,000 to 10,000 μg/kg, from 1,000 to 20,000 μg/kg, from 1,000 to 30,000 μg/kg, from 10,000 to 20,000 μg/kg, from 10,000 to 30,000 μg/kg, or from 20,000 to 30,000 μg/kg. In other examples, a dose can include 0.1 μg/kg, 1 μg /kg, 5 μg /kg, 10 μg /kg, 15 μg /kg, 20 μg /kg, 25 μg /kg, 30 μg /kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1,000 μg/kg, 1,500 μg/kg, 2,000 μg/kg, 2,500 μg/kg, 3,000 μg/kg, 3,500 μg/kg, 4,000 μg/kg, 4,500 μg/kg, 5,000 μg/kg, 5,500 μg/kg, 6,000 μg/kg, 6,500 μg/kg, 7,000 μg/kg, 7,500 μg/kg, 8,000 μg/kg, 8,500 μg/kg, 9,000 μg/kg, 9,500 μg/kg, 10,000 μg/kg, 10,500 μg/kg, 11,000 μg/kg, 11,500 μg/kg, 12,000 μg/kg, 12,500 μg/kg, 13,000 μg/kg, 13,500 μg/kg, 14,000 μg/kg, 14,500 μg/kg, 15,000 μg/kg, 15,500 μg/kg, 16,000 μg/kg, 16,500 μg/kg, 17,000 μg/kg, 17,500 μg/kg, 18,000 μg/kg, 18,500 μg/kg, 19,000 μg/kg, 19,500 μg/kg, 20,000 μg/kg, 20,500 μg/kg, 21,000 μg/kg, 21,500 μg/kg, 22,000 μg/kg, 22,500 μg/kg, 23,000 μg/kg, 23,500 μg/kg, 24,000 μg/kg, 24,500 μg/kg, 25,000 μg/kg, 25,500 μg/kg, 26,000 μg/kg, 26,500 μg/kg, 27,000 μg/kg, 27,500 μg/kg, 28,000 μg/kg, 28,500 μg/kg, 29,000 μg/kg, 29,500 μg/kg, 30,000 μg/kg, 30,500 μg/kg, 31,000 μg/kg, 31,500 μg/kg, 32,000 μg/kg, 32,500 μg/kg, 33,000 μg/kg, 33,500 μg/kg, 34,000 μg/kg, 34,500 μg/kg, 35,000 μg/kg, 35,500 μg/kg, 36,000 μg/kg, 36,500 μg/kg, 37,000 μg/kg, 37,500 μg/kg, 38,000 μg/kg, 38,500 μg/kg, 39,000 μg/kg, 39,500 μg/kg, or 40,000 μg/kg.

In other examples, a dose can include 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 150 mg/kg, 200 mg/kg, 250 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1000 mg/kg, or more.

Therapeutically effective amounts can be achieved by administering single or multiple doses during the course of a treatment regimen (e.g., weekly, every 2 weeks, every 3 weeks, monthly, every 2 months, every 3 months, every 4 months, every 5 months, every 6 months, every 7 months, every 8 months, every 9 months, every 10 months, every 11 months, or yearly. In particular embodiments, only a single administration is needed. In additional embodiments, administrations are provided every 30 days or every 60 days.

Additional information regarding appropriate methods of use for the depot formulations disclosed herein are found in International Patent Application Nos. PCT/US2014/020771 and PCT/US2014/047691, as well as U.S. patent application Ser. No. 14/124,669.

Depot formulations can be administered through any appropriate route including by injection; parenteral injection; instillation; or implantation into soft tissues, a body cavity, or occasionally into a blood vessel with injection through fine needles.

### EXAMPLES

The Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.
1. A depot formulation including: (i) an internal aqueous phase including a therapeutic protein, the therapeutic protein present at 0.025% to 5% w/w of the depot formulation; (ii) a polymer-based solid/oil phase; and (iii) an external aqueous phase in which particles are dispersed, the external aqueous phase including a surfactant present at 0.01% to 1% w/w of the depot formulation, wherein the depot formulation provides sustained release of the therapeutic protein within effective levels for at least one month following a single administration.
2. A depot formulation of embodiment 1, wherein the polymer is selected from poly(lactides), poly(glycolides), poly(lactide-co-glycolides), poly(lactic acid)s, poly(glycolic acid)s, poly(lactic acid-co-glycolic acid)s, poly(lactide-co-glycolide-graft PEG)s, or blends or copolymers thereof.
3. A depot formulation of embodiments 1 or 2, wherein the polymer is poly(lactide-co-glycolide) with a lactide:glycolide ratio of 1:1.
4. A depot formulation of any one of embodiments 1, 2, or 3, wherein the therapeutic protein is present at 0.025% w/w of the depot formulation; at 0.25% w/w of the depot formulation; or at 2.5% w/w of the depot formulation.
5. A depot formulation of any one of embodiments 1, 2, 3, or 4, wherein the surfactant is selected from polysorbates, poly(ethylene glycols), ethylene-propylene oxide (PEO-PPO) blends, poloxamers, Span®, Brij®, dioctyl-sulfosuccinate, poly(vinyl alcohol) (PVA), PVP, or combinations thereof.
6. A depot formulation of any one of embodiments 1, 2, 3, 4, or 5, wherein the therapeutic protein has at least 20 amino acids, at least 21 amino acids, at least 22 amino acids, at least 23 amino acids, at least 24 amino acids, at least 25 amino acids, at least 26 amino acids, at least 27 amino acids, at least 28 amino acids, at least 29 amino acids, at least 30 amino acids, at least 31 amino acids, at least 32 amino acids, at least 33 amino acids, at least 34 amino acids, at least 35 amino acids, at least 36 amino acids, at least 37 amino acids, at least 38 amino acids, at least 39 amino acids, at least 40 amino acids, at least 41 amino acids, at least 42 amino acids, at least 43 amino acids, at least 44 amino acids, at least 45 amino acids, at least 46 amino acids, at least 47 amino acids, at least 48 amino acids, at least 49 amino acids, at least 50 amino acids, at least 51 amino acids, at least 52 amino acids, at least 53 amino acids, at least 54 amino acids, or at least 55 amino acids.
7. A depot formulation of any one of embodiments 1, 2, 3, 4, or 5, wherein the therapeutic protein has 20 amino acids, 21 amino acids, 22 amino acids, 23 amino acids, 24 amino acids, 25 amino acids, 26 amino acids, 27 amino acids, 28 amino acids, 29 amino acids, 30 amino acids, 31 amino acids, 32 amino acids, 33 amino acids, 34 amino acids, 35 amino acids, 36 amino acids, 37 amino acids, 38 amino acids, 39 amino acids, 40 amino acids, 41 amino acids, 42 amino acids, 43 amino acids, 44 amino acids, 45 amino acids, 46 amino acids, 47 amino acids, 48 amino acids, 49 amino acids, 50 amino acids, 51 amino acids, 52 amino acids, 53 amino acids, 54 amino acids, or 55 amino acids.
8. A depot formulation of any one of embodiments 1, 2, 3, 4, 5, 6, or 7, wherein the therapeutic protein has at least one disulfide bridge, at least two disulfide bridges, at least three disulfide bridges, at least four disulfide bridges, or at least five disulfide bridges.
9. A depot formulation of any one of embodiments 1, 2, 3, 4, 5, 6, or 7, wherein the therapeutic protein has one disulfide bridge, two disulfide bridges, three disulfide bridges, four disulfide bridges, or five disulfide bridges.
10. A depot formulation of any one of embodiments 1, 2, 3, 4, 5, 6, 7, 8, or 9, wherein the therapeutic protein is a toxin-based therapeutic protein.
11. A depot formulation of any one of embodiments 1, 2, 3, 4, 5, 6, 7, 8, 9, or 10, wherein the therapeutic protein is an ShK-based protein.
12. A depot formulation of any one of embodiments 1, 2, 3, 4, 5, 6, 7, 8, 9, 10, or 11, wherein the therapeutic protein is an ShK-based protein that inhibits voltage-gated potassium channels.
13. A depot formulation of embodiment 12, wherein the inhibited voltage-gated potassium channels are one or more of Kv1.1, Kv1.3, Kv1.5, Kv1.3/1.5, Kv1.6, Kv3.2, or KCa3.1 channels.
14. A depot formulation of any one of embodiments 1, 2, 3, 4, 5, 6, 7, 8, 9, 10, 11, 12, or 13, wherein the therapeutic protein has a sequence of any one of SEQ ID NO:1-260.
15. A depot formulation of any one of embodiments 1, 2, 3, 4, 5, 6, 7, 8, 9, 10, 11, 12, 13, or 14, wherein the therapeutic protein has a sequence of any one of SEQ ID NO:1-224 or SEQ ID NO:257-260.
16. A depot formulation of any one of embodiments 1, 2, 3, 4, 5, 6, 7, 8, 9, 10, 11, 12, 13, 14, or 15, wherein the therapeutic protein is one or more of SEQ ID NO: 208, SEQ ID NO:217, SEQ ID NO:257, SEQ ID NO:210, SEQ ID NO:219, SEQ ID NO:218, SEQ ID NO:221, SEQ ID NO:258, SEQ ID NO:259, SEQ ID NO:260, or salts thereof.
17. A depot formulation of any one of embodiments 1, 2, 3, 4, 5, 6, 7, 8, 9, 10, 11, 12, 13, 14, 15, or 16, wherein the therapeutic protein is SEQ ID NO:217 or SEQ ID NO: 218.
18. A depot formulation of any one of embodiments 1, 2, 3, 4, 5, 6, 7, 8, 9, 10, 11, 12, 14, 15, or 16, wherein the therapeutic protein is SEQ ID NO:210.
19. A depot formulation of any one of embodiments 1, 2, 3, 4, 5, 6, 7, 8, 9, 10, 11, 12, 13, 14, 15, 16, 17, or 18, wherein the depot formulation provides sustained release of the therapeutic protein within effective levels for at least 40 days following a single administration, for at least 41 days following a single administration, for at least 42 days following a single administration, for at least 43 days following a single administration, for at least 44 days following a single administration, for at least 45 days following a single administration, for at least 46 days following a single administration, for at least 47 days following a single administration, for at least 48 days following a single administration, for at least 49 days following a single administration, for at least 50 days following a single administration, for at least 51 days following a single administration, for at least 52 days following a single administration, for at least 53 days following a single administration, for at least 54 days following a single administration, for at least 55 days following a single administration, or for at least 56 days following a single administration.
20. A depot formulation of any one of embodiments 1, 2, 3, 4, 5, 6, 7, 8, 9, 10, 11, 12, 13, 14, 15, 16, 17, or 18, wherein the depot formulation provides sustained release of the therapeutic protein within effective levels for 40 days following a single administration, for 41 days following a single administration, for 42 days following a single administration, for 43 days following a single administration, for 44 days following a single administration, for 45 days following a single administration, for 46 days following a single administration, for 47 days following a single administration, for 48 days following a single administration, for 49 days following a single administration, for 50 days following a single administration, for 51 days following a single administration, for 52 days following a single administration, for 53 days following a single administration, for 54 days following a single administration, for 55 days following a single administration, or for 56 days following a single administration.
21. A depot formulation of any one of embodiments 1, 2, 3, 4, 5, 6, 7, 8, 9, 10, 11, 12, 13, 14, 15, 16, 17, 18, or 19, wherein the depot formulation provides sustained release of the therapeutic protein within effective levels for at least two months following a single administration.
22. A depot formulation of any one of embodiments 1, 2, 3, 4, 5, 6, 7, 8, 9, 10, 11, 12, 13, 14, 15, 16, 17, or 18, wherein the depot formulation provides sustained release of the therapeutic protein within effective levels for two months following a single administration.
23. A depot formulation of any one of embodiments 1, 2, 3, 4, 5, 6, 7, 8, 9, 10, 11, 12, 13, 14, 15, 16, 17, 18, 19, or 21, wherein the depot formulation provides sustained release of the therapeutic protein within effective levels for at least three months following a single administration.
24. A depot formulation of any one of embodiments 1, 2, 3, 4, 5, 6, 7, 8, 9, 10, 11, 12, 13, 14, 15, 16, 17, 18, or 19, wherein the depot formulation provides sustained release of the therapeutic protein within effective levels for three months following a single administration.
25. A lyophilized depot formulation including: (i) a polymer-based external solid/oil phase including a therapeutic protein dispersed therein at 0.025% to 5% w/w of the lyophilized depot formulation; (ii) surfactants at 0.01% to 0.5% w/w of the lyophilized depot formulation; and (iii) sugar at 0.5 to 90% w/w of the lyophilized depot formulation, wherein after reconstitution of the lyophilized depot formulation the reconstituted depot formulation provides sustained release of the therapeutic protein within effective levels for at least one month following a single administration.
26. A lyophilized depot formulation of embodiment 25, wherein the sugar is sucrose, mannitol, trehalose, dextrose, or combinations thereof.
27. A lyophilized depot formulation of embodiments 25 or 26, wherein the sugar is sucrose.
28. A lyophilized depot formulation of any one of embodiments 25, 26, or 27, wherein the polymer is selected from poly(lactides), poly(glycolides), poly(lactide-co-glycolides), poly(lactic acid)s, poly(glycolic acid)s, poly(lactic acid-co-glycolic acid)s, poly(lactide-co-glycolide-graft PEG)s, or blends or copolymers thereof.
29. A lyophilized depot formulation of any one of embodiments 25, 26, 27, or 28, wherein the polymer is poly(lactide-co-glycolide) with a lactide:glycolide ratio of 1:1.
30. A lyophilized depot formulation of any one of embodiments 25, 26, 27, 28, or 29, wherein the therapeutic protein is present at 0.025% w/w of the lyophilized depot formulation; at 0.25% w/w of the lyophilized depot formulation; or at 2.5% w/w of the lyophilized depot formulation.
31. A lyophilized depot formulation of any one of embodiments 25, 26, 27, 28, 29, or 30, wherein the surfactant is selected from polysorbates, poly(ethylene glycols), ethylene-propylene oxide blends, poloxamers, Span®, Brij®, dioctyl-sulfosuccinate, PVA, PVP, or combinations thereof.
32. A lyophilized depot formulation of any one of embodiments 25, 26, 27, 28, 29, 30, or 31, wherein the therapeutic protein has at least 20 amino acids, at least 21 amino acids, at least 22 amino acids, at least 23 amino acids, at least 24 amino acids, at least 25 amino acids, at least 26 amino acids, at least 27 amino acids, at least 28 amino acids, at least 29 amino acids, at least 30 amino acids, at least 31 amino acids, at least 32 amino acids, at least 33 amino acids, at least 34 amino acids, at least 35 amino acids, at least 36 amino acids, at least 37 amino acids, at least 38 amino acids, at least 39 amino acids, at least 40 amino acids, at least 41 amino acids, at least 42 amino acids, at least 43 amino acids, at least 44 amino acids, at least 45 amino acids, at least 46 amino acids, at least 47 amino acids, at least 48 amino acids, at least 49 amino acids, at least 50 amino acids, at least 51 amino acids, at least 52 amino acids, at least 53 amino acids, at least 54 amino acids, or at least 55 amino acids.
33. A lyophilized depot formulation of any one of embodiments 25, 26, 27, 28, 29, 30, or 31, wherein the therapeutic protein has 20 amino acids, 21 amino acids, 22 amino acids, 23 amino acids, 24 amino acids, 25 amino acids, 26 amino acids, 27 amino acids, 28 amino acids, 29 amino acids, 30 amino acids, 31 amino acids, 32 amino acids, 33 amino acids, 34 amino acids, 35 amino acids, 36 amino acids, 37 amino acids, 38 amino acids, 39 amino acids, 40 amino acids, 41 amino acids, 42 amino acids, 43 amino acids, 44 amino acids, 45 amino acids, 46 amino acids, 47 amino acids, 48 amino acids, 49 amino acids, 50 amino acids, 51 amino acids, 52 amino acids, 53 amino acids, 54 amino acids, or 55 amino acids.
34. A lyophilized depot formulation of any one of embodiments 25, 26, 27, 28, 29, 30, 31, 32, or 33, wherein the therapeutic protein has at least one disulfide bridge, at least two disulfide bridges, at least three disulfide bridges, at least four disulfide bridges, or at least five disulfide bridges.
35. A lyophilized depot formulation of any one of embodiments 25, 26, 27, 28, 29, 30, 31, 32, or 33, wherein the therapeutic protein has one disulfide bridge, two disulfide bridges, three disulfide bridges, four disulfide bridges, or five disulfide bridges.
36. A lyophilized depot formulation of any one of embodiments 25, 26, 27, 28, 29, 30, 31, 32, 33, 34, or 35, wherein the therapeutic protein is a toxin-based therapeutic protein.
37. A lyophilized depot formulation of any one of embodiments 25, 26, 27, 28, 29, 30, 31, 32, 33, 34, or 35, wherein the therapeutic protein is an ShK-based protein.
38. A lyophilized depot formulation of embodiment 25, 26, 27, 28, 29, 30, 31, 32, 33, 34, or 35, wherein the therapeutic protein is an ShK-based protein that inhibits voltage-gated potassium channels.
39. A lyophilized depot formulation of embodiment 38, wherein the inhibited voltage-gated potassium channels are Kv1.1, Kv1.3, Kv1.5, Kv1.3/1.5, Kv1.6, Kv3.2, or KCa3.1 channels.
40. A lyophilized depot formulation of any one of embodiments 25, 26, 27, 28, 29, 30, 31, 32, 33, 34, 35, 36, 37, 38, or 39, wherein the therapeutic protein has a sequence of any one of SEQ ID NO:1-260.
41. A lyophilized depot formulation of any one of embodiments 25, 26, 27, 28, 29, 30, 31, 32, 33, 34, 35, 36, 37, 38, 39, or 40, wherein the therapeutic protein has a sequence of any one of SEQ ID NO:1-224 or SEQ ID NO:257-260.
42. A lyophilized depot formulation of any one of embodiments 25, 26, 27, 28, 29, 30, 31, 32, 33, 34, 35, 36, 37, 38, 39, 40, or 41, wherein the therapeutic protein is SEQ ID NO:208, SEQ ID NO:217, SEQ ID NO:257, SEQ ID NO:210, SEQ ID NO:219, SEQ ID NO:218, SEQ ID NO:221, or salts thereof.
43. A lyophilized depot formulation of any one of embodiments 25, 26, 27, 28, 29, 30, 31, 32, 33, 34, 35, 36, 37, 38, 39, 40, 41, or 42, wherein the reconstituted depot formulation provides sustained release of the therapeutic protein within effective levels for at least 40 days following a single administration, for at least 41 days following a single administration, for at least 42 days following a single administration, for at least 43 days following a single administration, for at least 44 days following a single administration, for at least 45 days following a single administration, for at least 46 days following a single administration, for at least 47 days following a single administration, for at least 48 days following a single administration, for at least 49 days following a single administration, for at least 50 days following a single administration, for at least 51 days following a single administration, for at least 52 days following a single administration, for at least 53 days following a single administration, for at least 54 days following a single administration, for at least 55 days following a single administration, or for at least 56 days following a single administration.
44. A lyophilized depot formulation of any one of embodiments 25, 26, 27, 28, 29, 30, 31, 32, 33, 34, 35, 36, 37, 38, 39, 40, 41, or 42, wherein the reconstituted depot formulation provides sustained release of the therapeutic protein within effective levels for 40 days following a single administration, for 41 days following a single administration, for 42 days following a single administration, for 43 days following a single administration, for 44 days following a single administration, for 45 days following a single administration, for 46 days following a single administration, for 47 days following a single administration, for 48 days following a single administration, for 49 days following a single administration, for 50 days following a single administration, for 51 days following a single administration, for 52 days following a single administration, for 53 days following a single administration, for 54 days following a single administration, for 55 days following a single administration, or for 56 days following a single administration.
45. A lyophilized depot formulation of any one of embodiments 25, 26, 27, 28, 29, 30, 31, 32, 33, 34, 35, 36, 37, 38, 39, 40, 41, 42, or 43, wherein the reconstituted depot formulation provides sustained release of the therapeutic protein within effective levels for at least two months following a single administration.
46. A lyophilized depot formulation of any one of embodiments 25, 26, 27, 28, 29, 30, 31, 32, 33, 34, 35, 36, 37, 38, 39, 40, 41, or 42, wherein the reconstituted depot formulation provides sustained release of the therapeutic protein within effective levels for two months following a single administration.
47. A lyophilized depot formulation of any one of embodiments 25, 26, 27, 28, 29, 30, 31, 32, 33, 34, 35, 36, 37, 38, 39, 40, 41, 42, 43, or 45, wherein the reconstituted depot formulation provides sustained release of the therapeutic protein within effective levels for at least three months following a single administration.
48. A lyophilized depot formulation of any one of embodiments 25, 26, 27, 28, 29, 30, 31, 32, 33, 34, 35, 36, 37, 38, 39, 40, 41, 42, or 43, wherein the reconstituted depot formulation provides sustained release of the therapeutic protein within effective levels for three months following a single administration.
49. A method of treating a subject having an autoimmune disease including administering to the subject a therapeutically effective amount of a depot formulation of any one of embodiments 1-48, thereby treating the subject.
50. A method of treating a subject having an autoimmune disease including administering a therapeutically effective amount of a depot formulation including a biocompatible polymer having a Kv1.3 channel inhibitor protein dispersed therein so as to be present at 0.0.25% to 5% w/w of the depot formulation, and a surfactant so as to be present at 0.1 to 1% w/w of the depot formulation, wherein the depot formulation is free from additional ingredients that alter the rate of release of the Kv1.3 channel inhibitor protein, thereby treating the subject.
51. A method of embodiments 49 or 50, wherein the autoimmune disease is multiple sclerosis, rheumatoid arthritis, psoriasis, psoriatic arthritis, lupus, lupus nephritis, organ transplant rejection, uveitis, dry eye disease, or autoimmune bowel disease.
52. A method of any one of embodiments 49, 50, or 51, wherein the administering is by injection.
53. A method of embodiment 52, wherein the injection is a single injection.
54. A depot formulation including: (i) a toxin-based therapeutic protein present at 1.2% w/w of the depot formulation; (ii) a PLG1A, PLG2A, PLG3A, PLG5E, or PLG7E polymer; and (iii) a PVA surfactant present at 0.1% w/w of the depot formulation wherein the depot formulation provides sustained release of the toxin-based therapeutic protein within effective levels for at least one month following a single administration.
55. A depot formulation including: (i) an internal aqueous phase including a toxin-based therapeutic protein, the toxin-based therapeutic protein present at 1.2% w/w of the depot formulation, and one or more buffers including phosphate, citrate, acetate, histidine, or combinations thereof, wherein the internal aqueous phase has a pH of 5.0-8.5; (ii) a PLG1A, 2A, 3A, 5E, or 7E polymer-based solid/oil phase; and (iii) an external aqueous phase including a PVA surfactant present at 0.01-0.10% w/w of the depot formulation, wherein the depot formulation provides sustained release of the toxin-based therapeutic protein within effective levels for at least one month following a single administration.
56. A depot formulation including: (i) a toxin-based therapeutic protein of SEQ ID NO: 1-260 present at 1.2% w/w of the depot formulation; (ii) a PLG1A, 2A, 3A, 5E, or 7E polymer; and (iii) a PVA surfactant present at 0.01-0.1% w/w of the depot formulation wherein the depot formulation provides sustained release of the toxin-based therapeutic protein within effective levels for at least one month following a single administration.
57. A depot formulation including: (i) an internal aqueous phase including a toxin-based therapeutic protein of SEQ ID NO: 1-260 present at 1.2% w/w of the depot formulation; one or more buffers including phosphate, citrate, acetate, histidine, or combinations thereof; and one or more salts including NaCl, KCl, CaCl₂, MgCl₂, (NH₄)₂CO₃, or combinations thereof, wherein the internal aqueous phase has a pH of 5.0-8.5; (ii) a PLG1A, 2A, 3A, 5E, or 7E polymer-based solid/oil phase; and (iii) an external aqueous phase including a PVA surfactant present at 0.01-0.1% w/w of the depot formulation, wherein the depot formulation provides sustained release of the toxin-based therapeutic protein within effective levels for at least one month following a single administration.
58. A method of obtaining sustained release of a therapeutic peptide in a subject comprising administering to the subject a depot formulation of any one of embodiments 1-48 or 54-57 thereby obtaining sustained release of the therapeutic peptide in the subject.
59. A method of embodiment 58 wherein the sustained release is evidenced by (1) release within effective levels for at least one month following a single administration; (2) release within effective levels wherein the Cₘₐₓ to C_{average} ratio does not exceed five or does not exceed three for at least one month following a single administration; (3) release within effective levels for at least 56 days following a single administration; and/or (4) release within effective levels wherein the Cₘₐₓ to C_{average} ratio does not exceed five or does not exceed three for at least 56 days following a single administration.

### Example 1

Preparation of a depot formulation of a therapeutic protein. Weights/volumes and proportions of polymer, solvent, aqueous phase, buffers, excipients, and co-solvents were as follows: 1.0 gram of polymer was dissolved in 5.0 mL dichloromethane. 0.5 mL of 100 mg/mL ShK-186 (or ShK-192) in 20 mM phosphate buffered saline (PBS; pH 6.0) was then added. The primary emulsion was homogenized for 2 minutes with a 10×195 mm probe, at 20,000 rpm. The primary emulsion was then added to an aqueous solution of 20 mM phosphate buffer (pH 6.0), 0.5% dioctylsulfosuccinate (DDS), and 0.05% PVA; and homogenized for 2 minutes with a 20×195 mm probe at 26,000 rpm. A stir bar was added to the double emulsion and the suspension stirred overnight in a fume hood to allow volatile solvent evaporation. The following day, the suspension was filtered through a 325 mesh screen. The filtered material was centrifuged down (20,000 g, 5 minutes) and the supernatant drawn off and analyzed by methods including high performance liquid chromatography (HPLC) and bicinchoninic acid (BCA) for protein (ShK-186 or ShK-192) content to determine encapsulation efficiency.

Polymers chosen to prepare different depot formulations assessed in this Example included low molecular weight (MW) PLG such as PLG1A, medium MW PLG such as PLG2A and high MW PLG such as PLG3A, all of which are hydrophilic, carboxy terminated (H series). Additional polymers tested were PLG5E and PLG7E, both of which are esterified (E series). Polymers used in this Example were purchased from Lakeshore Biomaterials.

### Example 2

Measurement of encapsulation, mass balance, and in vitro release of therapeutic protein from sustained released depot formulations. Percent encapsulation was typically measured immediately after depot formulation by assaying the free protein content of the supernatant following a separation method such as centrifugation and the use of equation (1):

% encapsulation=(total ShK−supernatant ShK)/total ShK

Typical encapsulation efficiencies were in the range of 60 to 90%.

Mass balance can be established by stressing the system to near-complete release of the ShK protein through heating above the polymer's glass transition temperature, addition of surfactant species such as concentrated SDDS, mechanical agitation, ultrasonication, alkaline or acid hydrolysis, or various combinations of these accelerated release methods. Typical in vitro release curves can be obtained by sampling the supernatant at various times and under different conditions and applying equation (2):

% release=supernatant ShK/total ShK

To measure in vitro release, the solutions were replaced with deionized water, 2% w/v surfactant SDDS (aq), 1× PBS, and 20 mM phosphate buffer (pH 7). The different samples were agitated on an orbital motion shake table and held at a temperature of 37° C. Release of protein over time was measured by BCA or reverse phase (RP)-HPLC analysis of supernatant drawn at various times, typically t=0 days to t=15 days.

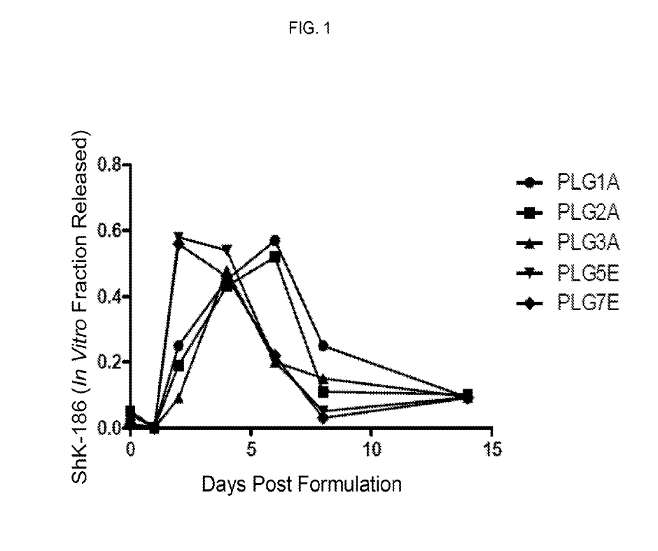

FIG. 1 shows in vitro release of ShK-186 of five different depot formulations. The formulations were made with a range of PLGs of different molecular weights and end capped chemistries as described in Example 1. The results show the effect of the terminal (end capped) group on modulating long term release, likely due to interaction with the therapeutic protein. The differential and absolute characteristics of such measurements can help establish in vitro/in vivo correlations as a functional test of formulation properties for product quality control. In this case, a more rapid initial release of therapeutic protein from the formulations made using the ester-capped polymers PLG5E and PLG7E is seen in vitro compared to the released observed from the carboxy-terminated based polymers (PLG1A, 2A, and 3A).

### Example 3

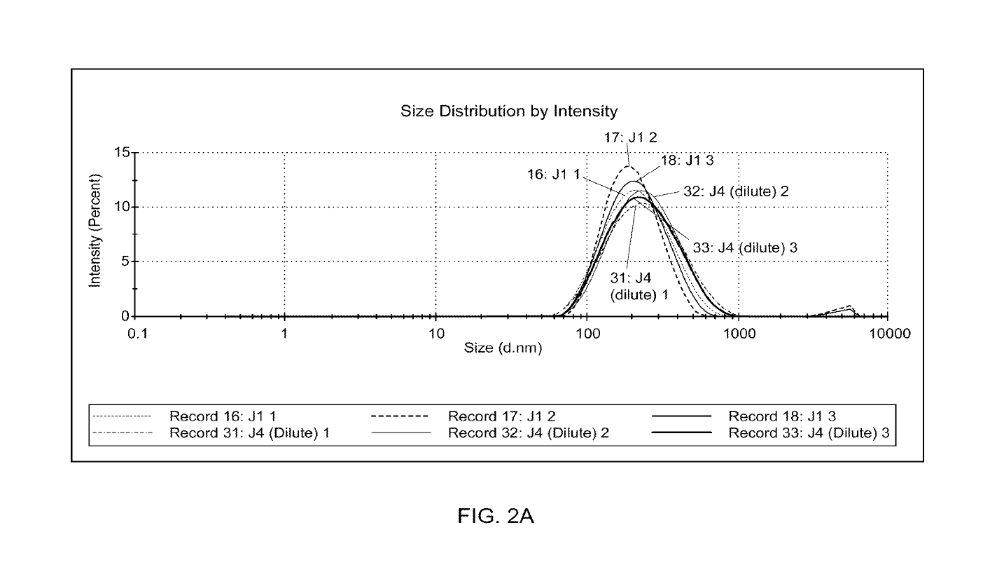

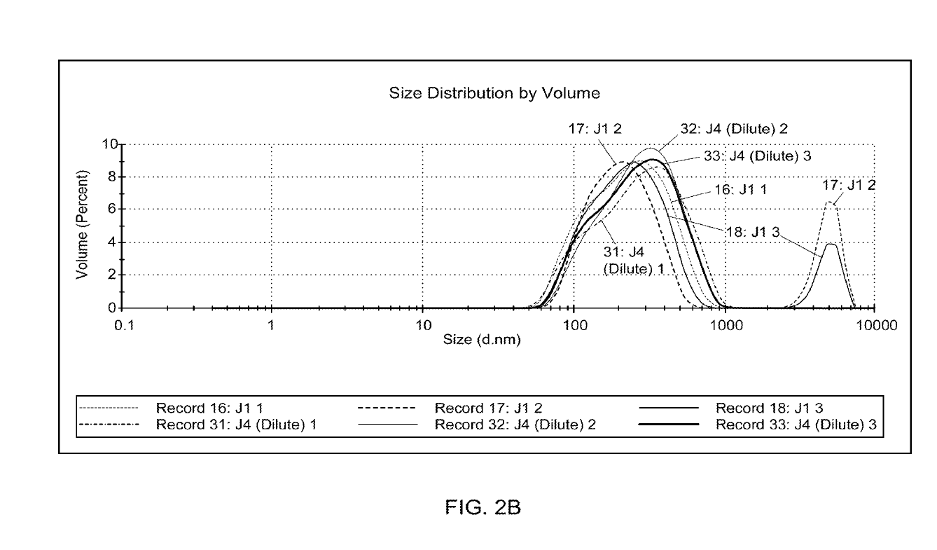

Characterization of the depot formulation physical properties. Standard analytical characterization methods were employed to analyze the physical and chemical properties of a depot formulation of ShK-186 disclosed herein and formed according to the methods of Example 1 using PLG2A polymer. Size and electrophoretic mobility were measured by light scattering using a Malvern nanosizer. Morphology/uniformity were measured by light microscopy. The results are shown in FIGS. 2-4.

FIGS. 2A and 2B show the dispersion size for three separate batches of depot formulations, as measured by dynamic light scattering. FIGS. 2A and 2B show size distributions of depot formulations plotted by intensity (FIG. 2A) and by volume (FIG. 2B) for formulation suspensions as measured by dynamic light scattering.

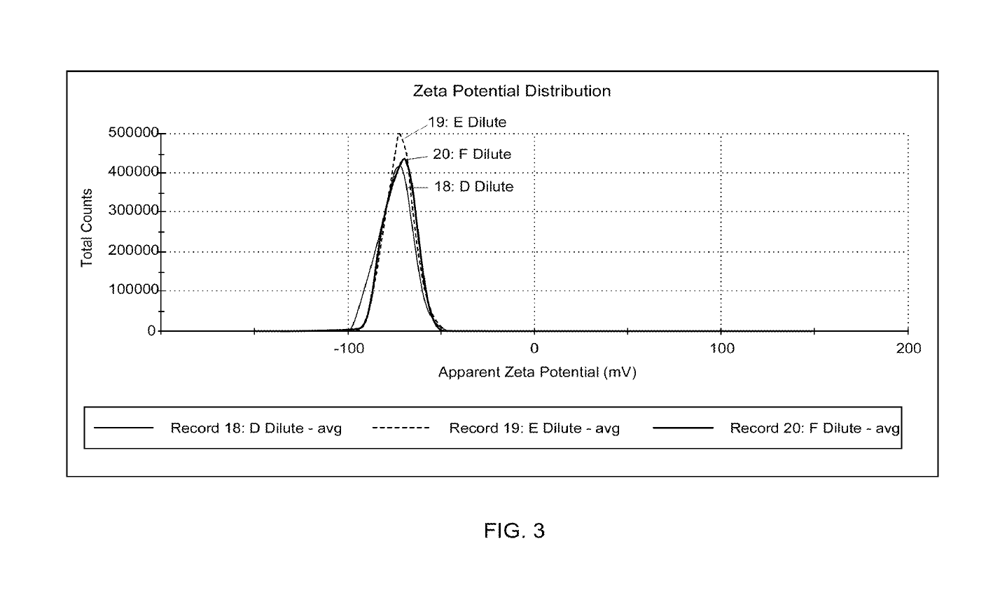

FIG. 3 is a measurement of the zeta potential (particle surface charge, as measured by electrophoretic mobility) for the depot formulations. The anionic surface layers help confer stability of the dispersion in aqueous suspensions. Zeta potential measurements showed similar, tight clustering of anionic particles with charges of −75, −72 and −72 mV, providing coulombic interactions that contribute to the colloidal stability through electrostatic repulsion.

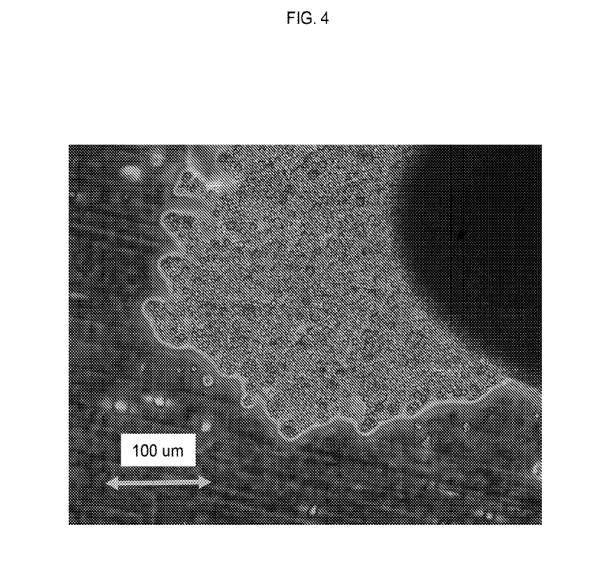

FIG. 4 is an optical microscope image of a depot formulation showing the shape of the PLG formulation and approximately uniform, geometric dimensions. The figure shows an optical microscopic (100X) image of a PLG formulation encapsulating ShK-186, showing round (presumably spherical) particles with a size of one micrometer.

In summary, this Example demonstrates that the depot formulations described in this Example have an average size of 1 micron (FIG. 4), that the surface/interfacial boundary of the spheres have a net electric charge of −75 mV-−72 mV as measured by electrophoretic mobility (FIG. 3) indicating colloidal stability, and that the formulations show uniform spherical geometries (FIG. 4).

### Example 4

Evaluation of depot formulations in vivo. The following process was followed to make the depot formulations described in this Example. 1.0 g of PLG polymer (1A, 2A or 3A) was dissolved into 5.0 mL dichloromethane with low speed stirring until the polymer was completely dissolved in a 10 mL beaker. 500 μL of ShK-186 (in a 20 mM sodium phosphate aqueous buffer (pH 6.0) with 140 mM NaCl, giving a final concentration of 20 mg/mL protein) was added and homogenized for 2 minutes with a 10×195 mm probe at 26,000 rpm. All of the w/o emulsion was poured into a 100 mL Pyrex® (Corning, Inc., Corning, N.Y.) 1395 media bottle charged with 20 mL of 0.5% w/w DSS, 20 mM phosphate (pH 7.0), and 0.05% w/w PVA water solution.

The mixture was homogenized for 5 minutes at the same shear setting (26,000 rpm), with the beaker in thermal contact with melting ice (0° C.). The generator probe was kept fully immersed in the liquid to limit frothing and spillage of material. The emulsion turned a milky white color due to colloidal scattering. The aqueous (external) phase pH was verified and adjusted if necessary to 5<pH<7.5. The formulation was removed from shear, stirred overnight at room temperature in a fume hood to allow solvent evaporation (dichloromethane). The media bottle was lightly covered to avoid excessive evaporation of water overnight.

The next day, the suspension was filtered through a 325 mesh screen. Very little (<5%) solid material was removed by the mesh screen. The suspension was then stored at room temperature with end over end mixing to avoid settling/clumping. Percent encapsulation was measured through centrifugation of the suspension, drawing the supernatant phase and assaying for ShK-186 content by methods such as BCA or HPLC. Encapsulation efficiencies were 88%, 87%, and 82% for PLG1A, 2A, and 3A, respectively.

Immediately before use by injection, the formulations were centrifuged utilizing the appropriate volume for 8 min, 4° C. at 2,000 g. The solid particles settled to the bottom, allowing a decanting of the supernatant that was almost clear or slightly hazy, followed by replacement of aqueous solution with an appropriate volume and composition (buffer, pH, and ionic strength), followed by mechanical mixing to resuspend the particles (vortexing if necessary) to obtain a uniform, free flowing dispersion (milky white in color).

Sprague Dawley rats (males ages 8-16 weeks, n=1 per condition) were given a SC injection with 1 mL of the indicated depot formulations using a 23-gauge needle. Blood was drawn at various time points post injection and assayed for ShK-186 by enzyme-linked immunosorbent assay (ELISA).

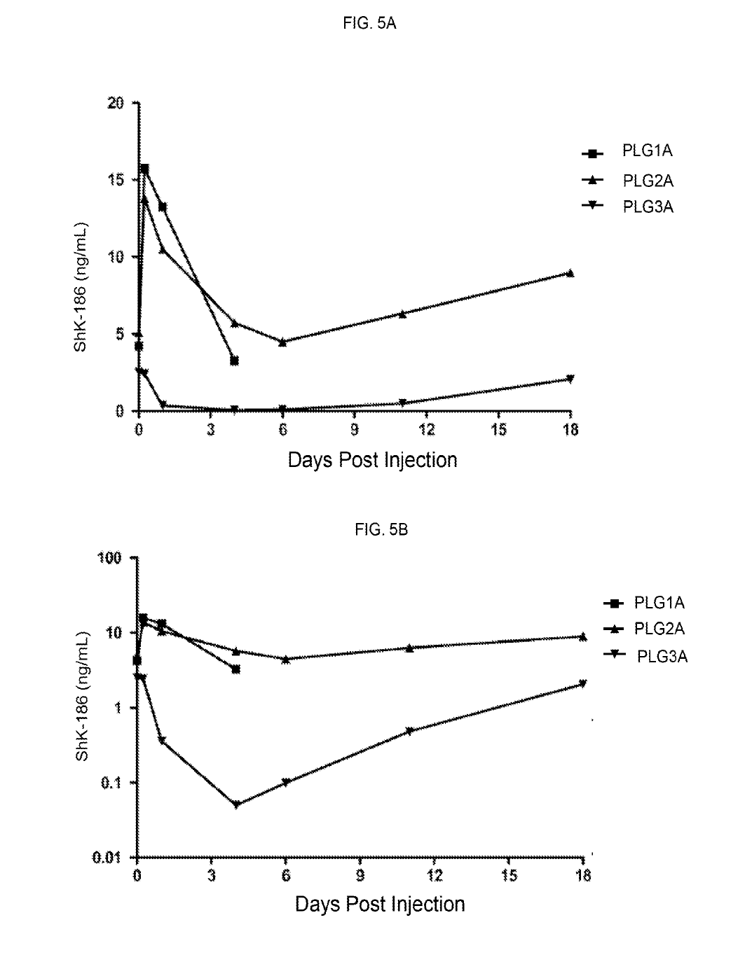

Examples of in vivo release profiles from depot formulations made following this procedure are shown in FIG. 5A (linear concentrations) and FIG. 5B (Log concentrations). In contrast to a saline solution of the therapeutic protein that reached a Cₘₐₓ within 15 minutes then constantly decreased and was eliminated to very low levels within a day, depot formulations formed using ShK-186 and PLG polymers 1A and 2A resulted in detectable protein at 4 days, and that of PLG2A was also detectable until day 18. The PLG3A-based formulation resulted in lower levels of sustained release of drug in vivo.

### Example 5

In vivo dose response of depot formulations using ShK-186 and ShK-192 with PLG2A. Preparation of depot formulations was performed as follows: 1.0 g of PLG2A was dissolved into 5.0 mL dichloromethane with mechanical stirring until the polymer was completely dissolved. A volume of 500 μL including three levels of ShK-186 (40, 20, and 10 mg/mL final concentration; in a 20 mM sodium phosphate aqueous buffer (pH 6.0) with 140 mM NaCl) was added separately into three separate PLG2A preparations and homogenized for 2 minutes with a 10×195 mm probe (26,000 rpm). The w/o emulsion was then poured into a 100 mL Pyrex® 1395 media bottle charged with 20 mL of 0.5% w/w DSS, and 20 mM phosphate (pH 7.0) water solution. The mixture was homogenized for 5 minutes at the same shear setting (26,000 rpm), with the beaker in thermal contact with melting ice (0° C.). The pH of the aqueous (external) phase was verified and adjusted if necessary to 5.0<pH<7.5.

The formulation was then removed from shear, and stirred overnight at room temperature in a fume hood to allow solvent evaporation. The next morning the suspension was filtered through a 325 mesh screen and stored at room temperature with end over end mixing to avoid settling/clumping. Immediately before use by injection, the formulations were centrifuged utilizing the appropriate volume for 8 min, 4° C. at 2,000 g. The solid particles settled to the bottom, allowing for decanting of the almost clear supernatant, followed by replacement of aqueous solution with an appropriate volume and composition (e.g. PBS), followed by mechanical mixing to resuspend the particles.

Sprague Dawley rats (females, ages 8-16 weeks, n=3) were given a SC injection of 1 mL of the indicated depot formulations. Blood was drawn at various time points and assayed for ShK-186 by ELISA methods.

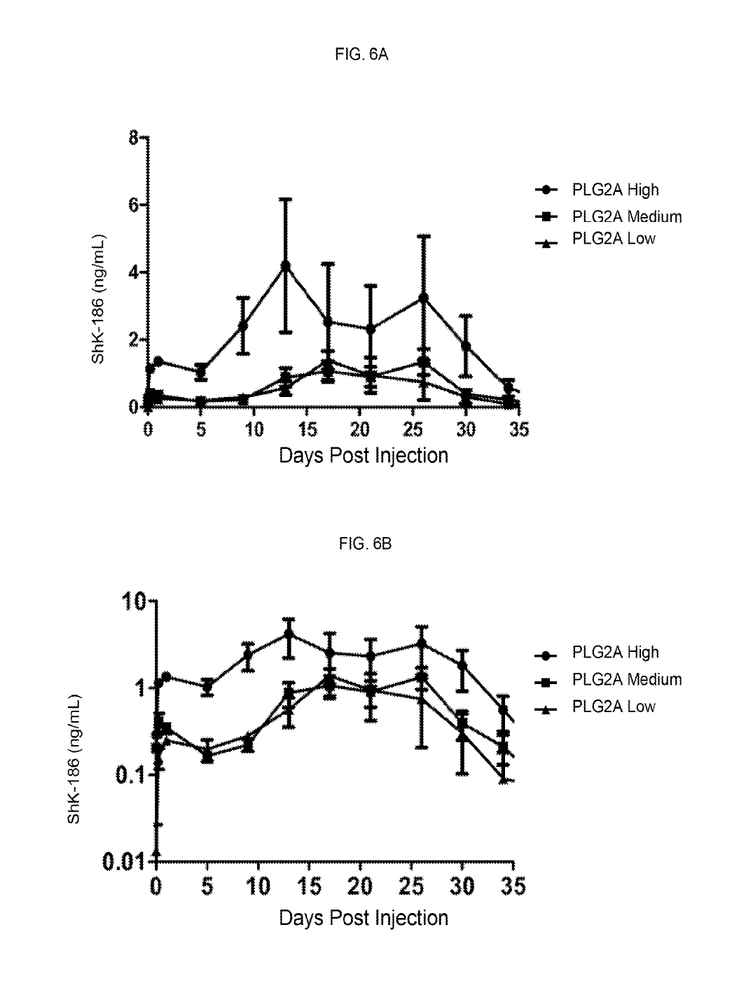

FIGS. 6A and 6B show in vivo release for different doses (high=40,000 μg/kg, medium=20,000 μg/kg, low=10,000 μg/kg) of ShK-186 formulated with PLG2A (FIG. 6A shows linear concentrations and FIG. 6B shows Log concentrations). The blood serum levels of ShK-186 are maintained for more than 30 days over a relatively narrow range of concentrations. As shown in FIGS. 6A and 6B, the relative shapes of the release profiles are geometrically similar, but scaled by the area under the curve to the total dose of ShK-186. These results demonstrate that a long acting depot formulations could be formulated to keep circulating levels of biologically efficacious and intact ShK-186 for extended periods of time.

The next part of this Example was performed to test formulation and in vivo release of ShK-192 over extended time durations. Similar to the methods used for ShK-186, 1.0 g of PLG2A was dissolved into 5.0 mL dichloromethane with mechanical stirring until the polymer was completely dissolved. A volume of 500 μL including a low dose of ShK-192 (10 mg/mL final concentration; in a 20 mM sodium phosphate aqueous buffer (pH 6.0) with 140 mM NaCl) was formulated by homogenizing for 2 minutes with a 10×195 mm probe (26,000 rpm). The w/o emulsion was then transferred into a 100 mL Pyrex® 1395 media bottle charged with 20 mL of 0.5% w/w DSS, 20 mM phosphate (pH 7.0), water solution.

The mixture was held in thermal contact with ice water (0° C.) and homogenized for 5 minutes at 26,000 rpm, with the final pH of the aqueous (external) phase verified and adjusted to 5.0<pH<7.5. The formulation was then removed from homogenization and mechanically stirred overnight at room temperature in a fume hood to permit solvent evaporation. The next day the suspension was first filtered through a 325 mesh screen, then stored at room temperature with end over end mixing to minimize settling/clumping. Immediately before in vivo use, the formulations were centrifuged utilizing the appropriate volume for 8 minutes, at 4° C. at 2,000 g. The solid particles separated to the bottom, allowing for decanting of the almost clear supernatant, followed by replacement of aqueous solution with an appropriate volume and composition of aqueous buffer (e.g. PBS), followed by mechanical mixing to resuspend the particles. The suspension (1 mL) was then drawn up and administered in a SC injection to rats (n=3) using a 23-gauge needle. Blood was drawn at various time points and assayed for ShK-192 using an ELISA assay.

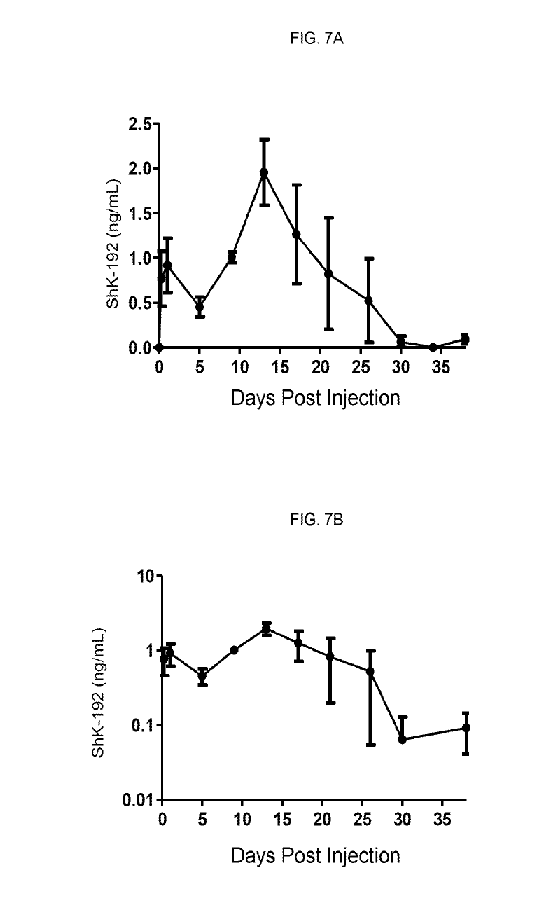

FIGS. 7A and 7B are plots of in vivo release for ShK-192 dosed at 10,000 μg/kg in Sprague-Dawley rats, following a single, SC injection (FIG. 7A shows linear concentrations and FIG. 7B shows Log concentrations). The maximum concentration was reached near t=12 days suggesting a gradual release of the therapeutic protein from the depot formulation, a process that continues relatively smoothly for over 30 days. In summary, FIG. 7 shows the time course of blood serum levels for ShK-192 in Sprague-Dawley rats, with standard deviations plotted as the y-axis error limits. This graph demonstrates that different biomolecularly active Kv1.3 channel inhibitors were formulated to give sustained release and efficacious blood serum concentrations over an extended period of more than one month.

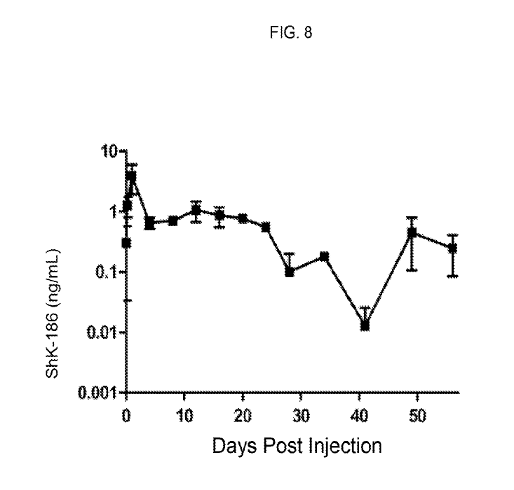

In a repeat experiment, the time during which blood levels of ShK-186 were monitored was extended. As can be seen in FIG. 8, in vivo release of ShK-186 formulated with PLG2A in a single SC dose (1 mL) at 20,000 μg/kg in Sprague-Dawley rats was sustained to at least 56 days.

### Example 6

Therapeutic efficacy of ShK-186 and ShK-192 depot formulations in animal models of autoimmune disease including the delayed type hypersensitivity rat model. Lewis rats are vaccinated by SC administration to the base of the tail with 100 μg ovalbumin mixed 1:1 (v/v) in Complete Freund's Adjuvant (CFA) (OVA/CFA, 200 μL volume) on day 0 using a 20 G×1% needle. Delayed-type hypersensitivity (DTH) is elicited in the ear on Day 7 by injection of 20 μg OVA in 10 μL to the pinna of the ear of isoflurane-anesthetized animals using 29 G×1 needles. DTH is evaluated on Days 8 and 9 by measuring the induration of the site of antigen injection with a micrometer (caliper). Control treatments with ShK-186 or ShK-192 include SC injections of ShK-186 or ShK-192 at 100 μg/kg, 10 μg/kg, 3 μg/kg or 1 μg/kg given on day 0-7 (daily from the initial immunization). The control treatments are compared to a single injection of a depot formulation of ShK-186 or ShK-192 using a PLG polymer-based (in one example, PLG2A) sustained formulations at a dose of 1,000 μg/kg, 5,000 μg/kg, 10,000 μg/kg, 20,000 μg/kg, or 40,000 μg/kg given on day 0 (time of immunization).

The depot formulations of ShK-186 or ShK-192 are expected to show a significant therapeutic effect when given as a single injection in that both will reduce the induration (inflammatory response) to levels comparable to or further than those obtained as a result of daily SC injection of ShK-186 or ShK-192 in buffer P6N (10 mM sodium phosphate, 0.8% (w/v) NaCl, 0.05% (w/v) polysorbate 20, in water for injection, pH=5).

### Example 7

Therapeutic efficacy of ShK-186 and ShK-192 depot formulations in animal models of autoimmune disease including the chronic relapsing/remitting autoimmune encephalomyelitis rat model. Chronic relapsing/remitting autoimmune encephalomyelitis (CR-EAE) is induced by SC injection of spinal cord homogenate (Bioreclamation, Inc.) and CFA to dark agouti rats (Harlan). Animals typically recover by 30 days after onset of CR-EAE. Rats are treated with different amounts of ShK-186 or ShK-192 (1, 3, 5, 10, and 100 μg/kg) in SC injections daily, every two days, or every three days at different time points prior to and after elicitation of CR-EAE by immunization. These regimens are compared to single injection of depot formulations (ShK-186 or ShK-192 with PLG2A) at doses of 1,000 μg/kg, 5,000 μg/kg, 10,000 μg/kg, 20,000 μg/kg, or 40,000 μg/kg given at the beginning of the experiment (time immunization, prevention model) and after onset of disease, i.e., after a rat has a clinical score of 1 or greater (treatment model).

The efficacy of the treatments is measured by clinical scoring of the severity of CR-EAE in daily and depot formulation-treated rats. Disease is monitored and scored twice daily for a set period of time after SC injection of spinal cord homogenate/CFA emulsions using the following scoring system: (0) no disease; (0.5) distal limp tail; (1) limp tail; (2) mild paraparesis, ataxia; (3) moderate paraparesis, the rats trips from time to time; (3.5) one hind limb is paralyzed, the other moves; (4) complete hind limb paralysis; (5) complete hind limb paralysis and incontinence; and (6) moribund, difficulty breathing, does not eat or drink/euthanize immediately. Subsets of rats are sacrificed at specific time points of the experiment to harvest tissues or to collect whole blood samples.

The depot formulations of ShK-186 or ShK-192 are expected to show significant therapeutic effects, e.g., reduction in the clinical scores, when given as a single injection in both the prevention and treatment models. These effects are expected to be comparable to or better than the effects observed by the different drug administration regimens tested when ShK-186 or ShK-192 are given daily in buffer P6N.

### Example 8

Treatment of Psoriasis. The autoimmune disease psoriasis is an autoimmune disease where effector memory T cells have been shown to be implicated in disease and express the target of ShK-186, the potassium channel, Kv1.3. Psoriasis patients are dosed once with a depot formulation disclosed herein and then evaluated at different time points post dosing to monitor the therapeutic effects of the therapeutic protein (e.g., ShK-186, ShK-192). Doses to be evaluated include 0.1, 1, 10, 100, 1000, 10,000, 20,000, or 30,000 mcg/Kg. Time points for evaluation include 1, 2, 3, 4, 6, 8, 12, 16, 24 weeks post dosing. To evaluate therapeutic protein exposure following a single injection of the depot formulation, blood samples from the patients are collected at desired time points post injection and the amount of drug in circulation is detected by established bioanalytical methods. Clinical scores of psoriasis are also monitored. Sustained release of the therapeutic protein(s) and improved clinical scores are expected.

As will be understood by one of ordinary skill in the art, each embodiment disclosed herein can comprise, consist essentially of, or consist of its particular stated element, step, ingredient or component. Thus, the terms “include” or “including” should be interpreted to recite: “comprise, consist of, or consist essentially of.” The transitional term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would prevent the particular embodiment from achieving sustained release of a therapeutic protein as “sustained release” is defined herein.

Unless otherwise indicated, all numbers used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently includes certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to include the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to publications, patents and/or patent applications (collectively “references”) throughout this specification. Each of the cited references is individually incorporated herein by reference for their particular cited teachings.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A depot formulation consisting of:
(i) a particle consisting of:
(a) an internal aqueous phase comprising a therapeutic protein of SEQ ID NO: 217 or SEQ ID NO: 218, a buffer, and a salt, wherein the internal aqueous phase of the particle has a pH of 6.0-8.5; and
(b) a polymer phase comprising poly(lactide-co-glycolide) (PLG)2A; and
(ii) an aqueous phase surrounding the particle comprising a poly(vinyl alcohol) (PVA) surfactant;
wherein the depot formulation provides sustained release of the therapeutic protein within effective levels for at least one month following a single administration.

2. A depot formulation consisting of:
(i) a particle consisting of:
(a) an internal aqueous phase comprising a therapeutic protein of SEQ ID NO: 217 or SEQ ID NO: 218, a buffer, and a salt, wherein the internal aqueous phase of the particle has a pH of 6.0-8.5, and
(b) a polymer phase comprising a carboxy-terminated medium molecular weight PLG; and
(ii) an aqueous phase surrounding the particle comprising a surfactant;
wherein the depot formulation provides sustained release of the therapeutic protein within effective levels for at least one month following a single administration.

3. A depot formulation consisting of:
(i) a particle consisting of:
(a) an internal aqueous phase comprising a therapeutic protein with at least one disulfide bridge, at least two disulfide bridges, or at least three disulfide bridges, a buffer, and a salt, wherein the internal aqueous phase of the particle has a pH of 6.0-8.5, and
(b) a polymer phase comprising PLG2A; and
(ii) an aqueous phase surrounding the particle comprising a PVA surfactant;
wherein the depot formulation provides sustained release of the therapeutic protein within effective levels for at least one month following a single administration.

4. A depot formulation consisting of:
(i) a particle consisting of:
(a) an internal aqueous phase comprising a therapeutic protein with at least one disulfide bridge, at least two disulfide bridges, or at least three disulfide bridges, a buffer, and a salt, wherein the internal aqueous phase of the particle has a pH of 6.0-8.5, and
(b) a polymer phase comprising a carboxy-terminated medium molecular weight PLG; and
(ii) an aqueous phase surrounding the particle comprising a surfactant;
wherein the depot formulation provides sustained release of the therapeutic protein within effective levels for at least one month following a single administration.

5. A depot formulation consisting essentially of:
(i) an internal aqueous phase comprising:
(a) a toxin-based therapeutic protein of any one of SEQ ID NO: 1-260 present at 1.2% w/w of the depot formulation,
(b) a buffer comprising phosphate, citrate, acetate, histidine, or combinations thereof, and
(c) a salt selected from NaCl, KCl, CaCl₂, MgCl₂, (NH₄)₂CO₃, or combinations thereof,
wherein the internal aqueous phase has a pH of 5.0-8.5;
(ii) a carboxy-terminated medium molecular weight PLG polymer-based solid/oil phase; and
(iii) an external aqueous phase comprising a PVA surfactant present at 0.01-0.1% w/w of the depot formulation,
wherein the depot formulation provides sustained release of the toxin-based therapeutic protein within effective levels for at least one month following a single administration.

6. A depot formulation consisting essentially of:
(i) an internal aqueous phase comprising:
(a) a toxin-based therapeutic protein of any one of SEQ ID NO: 1-260 present at 1.2% w/w of the depot formulation,
(b) a buffer comprising phosphate, citrate, acetate, histidine, or combinations thereof, and
(c) a salt selected from NaCl, KCl, CaCl₂, MgCl₂, (NH₄)₂CO₃, or combinations thereof,
wherein the internal aqueous phase has a pH of 5.0-8.5;
(ii) a PLG2A polymer-based solid/oil phase; and
(iii) an external aqueous phase comprising a PVA surfactant present at 0.01-0.1% w/w of the depot formulation,
wherein the depot formulation provides sustained release of the toxin-based therapeutic protein within effective levels for at least one month following a single administration.

7. A depot formulation consisting essentially of: (i) a toxin-based therapeutic protein of any one of SEQ ID NO: 1-260 present at 1.2% w/w of the depot formulation; (ii) a PLG2A polymer; and (iii) a PVA surfactant present at 0.01-0.1% w/w of the depot formulation; wherein the depot formulation provides sustained release of the toxin-based therapeutic protein within effective levels for at least one month following a single administration.

8. A depot formulation consisting essentially of:
(i) an internal aqueous phase comprising:
(a) a toxin-based therapeutic protein present at 1.2% w/w of the depot formulation, and
(b) a buffer comprising phosphate, citrate, acetate, histidine, or combinations thereof,
wherein the internal aqueous phase has a pH of 5.0-8.5;
(ii) a PLG2A polymer-based solid/oil phase; and
(iii) an external aqueous phase comprising a PVA surfactant present at 0.01-0.10% w/w of the depot formulation,
wherein the depot formulation provides sustained release of the toxin-based therapeutic protein within effective levels for at least one month following a single administration.

9. A depot formulation consisting essentially of: (i) a toxin-based therapeutic protein; (ii) a PLG2A polymer; and (iii) a PVA surfactant, wherein the depot formulation provides sustained release of the toxin-based therapeutic protein within effective levels for at least one month following a single administration.

10. A depot formulation consisting essentially of: (i) an internal aqueous phase comprising a therapeutic protein present at 0.025% to 5% w/w of the depot formulation; (ii) a polymer-based solid/oil phase; and (iii) an external aqueous phase comprising a surfactant present at 0.01% to 1% w/w of the depot formulation, wherein the depot formulation provides sustained release of the therapeutic protein within effective levels for at least one month following a single administration.

11. A depot formulation of claim 10, wherein the polymer is selected from poly(lactides), poly(glycolides), PLGs, poly(lactic acid)s, poly(glycolic acid)s, poly(lactic acid-co-glycolic acid)s, PLG-graft polyethylene glycol (PEG)s, or blends or copolymers thereof.

12. A depot formulation of claim 11, wherein the polymer is PLG with a lactide:glycolide ratio of 1:1.

13. A depot formulation of claim 10, wherein the therapeutic protein is present at 0.025% w/w of the depot formulation; at 0.25% w/w of the depot formulation; or at 2.5% w/w of the depot formulation.

14. A depot formulation of claim 10, wherein the surfactant is selected from polysorbates, PEG, ethylene-propylene oxide (PEO-PPO) blends, poloxamers, dioctyl-sulfosuccinate, PVA, Polyvinylpyrrolidone (PVP), or combinations thereof.

15. A depot formulation of claim 10, wherein the therapeutic protein has at least 20 amino acids, at least 21 amino acids, at least 22 amino acids, at least 23 amino acids, at least 24 amino acids, at least 25 amino acids, at least 26 amino acids, at least 27 amino acids, at least 28 amino acids, at least 29 amino acids, at least 30 amino acids, at least 31 amino acids, at least 32 amino acids, at least 33 amino acids, at least 34 amino acids, at least 35 amino acids, at least 36 amino acids, at least 37 amino acids, at least 38 amino acids, at least 39 amino acids, at least 40 amino acids, at least 41 amino acids, at least 42 amino acids, at least 43 amino acids, at least 44 amino acids, at least 45 amino acids, at least 46 amino acids, at least 47 amino acids, at least 48 amino acids, at least 49 amino acids, at least 50 amino acids, at least 51 amino acids, at least 52 amino acids, at least 53 amino acids, at least 54 amino acids, or at least 55 amino acids.

16. A depot formulation of claim 10, wherein the therapeutic protein has at least one disulfide bridge, at least two disulfide bridges, at least three disulfide bridges, at least four disulfide bridges, or at least five disulfide bridges.

17. A depot formulation of claim 10, wherein the therapeutic protein is a toxin-based therapeutic protein.

18. A depot formulation of claim 10, wherein the therapeutic protein is an ShK-based protein that inhibits voltage-gated potassium channels.

19. A depot formulation of claim 18, wherein the inhibited voltage-gated potassium channels are Kv1.1, Kv1.3, Kv1.5, Kv1.3/1.5, Kv1.6, Kv3.2, or KCa3.1 channels.

20. A depot formulation of claim 10, wherein the therapeutic protein has a sequence of any one of SEQ ID NO:1-260.

21. A depot formulation of claim 10, wherein the therapeutic protein is SEQ ID NO:208, SEQ ID NO:217, SEQ ID NO:257, SEQ ID NO:210, SEQ ID NO:219, SEQ ID NO:218, SEQ ID NO:221, or salts thereof.

22. A depot formulation of claim 21, wherein the therapeutic protein is SEQ ID NO:217.

23. A depot formulation of claim 21, wherein the therapeutic protein is SEQ ID NO:218.

24. A depot formulation of claim 21, wherein the therapeutic protein is SEQ ID NO:210.

25. A depot formulation of claim 10, wherein the depot formulation provides sustained release of the therapeutic protein within effective levels for at least 40 days, for at least 41 days, for at least 42 days, for at least 43 days, for at least 44 days, for at least 45 days, for at least 46 days, for at least 47 days, for at least 48 days, for at least 49 days, for at least 50 days, for at least 51 days, for at least 52 days, for at least 53 days, for at least 54 days, for at least 55 days, or for at least 56 days following a single administration.

26. A depot formulation of claim 10, wherein the polymer is PLG1A, PLG2A, PLG3A, PLG5E, or PLG7E.

27. A lyophilized depot formulation consisting essentially of: (i) a polymer-based solid/oil phase comprising a therapeutic protein dispersed therein at 0.025% w/w to 5% w/w of the lyophilized depot formulation; (ii) surfactants at 0.01% to 0.5% w/w of the lyophilized depot formulation; and (iii) sugar at 0.5 to 90% w/w of the lyophilized depot formulation, wherein after reconstitution of the lyophilized depot formulation, the reconstituted depot formulation provides sustained release of the therapeutic protein within effective levels for at least one month following a single administration.

28. A lyophilized depot formulation of claim 27, wherein the sugar is sucrose, mannitol, trehalose, dextrose, or combinations thereof.

29. A lyophilized depot formulation of claim 28, wherein the sugar is sucrose.

30. A lyophilized depot formulation of claim 27, wherein the polymer is selected from poly(lactides), poly(glycolides), PLG, poly(lactic acid)s, poly(glycolic acid)s, poly(lactic acid-co-glycolic acid)s, PLG-graft PEGs, or blends or copolymers thereof.

31. A lyophilized depot formulation of claim 30, wherein the polymer is PLG with a lactide:glycolide ratio of 1:1.

32. A lyophilized depot formulation of claim 27, wherein the therapeutic protein is present at 0.025% w/w of the lyophilized depot formulation; at 0.25% w/w of the lyophilized depot formulation; or at 2.5% w/w of the lyophilized depot formulation.

33. A lyophilized depot formulation of claim 27, wherein the surfactant is selected from polysorbates, PEGs, ethylene-propylene oxide blends, poloxamers, dioctyl-sulfosuccinate, PVA, PVP, or combinations thereof.

34. A lyophilized depot formulation of claim 27, wherein the therapeutic protein has at least 20 amino acids, at least 21 amino acids, at least 22 amino acids, at least 23 amino acids, at least 24 amino acids, at least 25 amino acids, at least 26 amino acids, at least 27 amino acids, at least 28 amino acids, at least 29 amino acids, at least 30 amino acids, at least 31 amino acids, at least 32 amino acids, at least 33 amino acids, at least 34 amino acids, at least 35 amino acids, at least 36 amino acids, at least 37 amino acids, at least 38 amino acids, at least 39 amino acids, at least 40 amino acids, at least 41 amino acids, at least 42 amino acids, at least 43 amino acids, at least 44 amino acids, at least 45 amino acids, at least 46 amino acids, at least 47 amino acids, at least 48 amino acids, at least 49 amino acids, at least 50 amino acids, at least 51 amino acids, at least 52 amino acids, at least 53 amino acids, at least 54 amino acids, or at least 55 amino acids.

35. A lyophilized depot formulation of claim 27, wherein the therapeutic protein has at least one disulfide bridge, at least two disulfide bridges, at least three disulfide bridges, at least four disulfide bridges, or at least five disulfide bridges.

36. A lyophilized depot formulation of claim 27, wherein the therapeutic protein is a toxin-based therapeutic protein.

37. A lyophilized depot formulation of claim 27, wherein the therapeutic protein is an ShK-based protein that inhibits voltage-gated potassium channels.

38. A lyophilized depot formulation of claim 37, wherein the inhibited voltage-gated potassium channels are Kv1.1, Kv1.3, Kv1.5, Kv1.3/1.5, Kv1.6, Kv3.2, or KCa3.1 channels.

39. A lyophilized depot formulation of claim 27, wherein the therapeutic protein has a sequence of any one of SEQ ID NO:1-260.

40. A lyophilized depot formulation of claim 27, wherein the therapeutic protein is SEQ ID NO:208, SEQ ID NO:217, SEQ ID NO:257, SEQ ID NO:210, SEQ ID NO:219, SEQ ID NO:218, SEQ ID NO:221, or salts thereof.

41. A lyophilized depot formulation of claim 27, wherein the reconstituted depot formulation provides sustained release of the therapeutic protein within effective levels for at least 40 days, for at least 41 days, for at least 42 days, for at least 43 days, for at least 44 days, for at least 45 days, for at least 46 days, for at least 47 days, for at least 48 days, for at least 49 days, for at least 50 days, for at least 51 days, for at least 52 days, for at least 53 days, for at least 54 days, for at least 55 days, or for at least 56 days following a single administration.

42. A lyophilized depot formulation of claim 27, wherein the polymer is PLG1A, PLG2A, PLG3A, PLG5E, or PLG7E.

43. A method of obtaining sustained release of a therapeutic protein in a subject comprising administering to the subject a depot formulation of any one of claims 1-42 thereby obtaining sustained release of the therapeutic protein in the subject.

44. A method of claim 43 wherein the sustained release is evidenced by (1) release within effective levels for at least one month following a single administration; (2) release within effective levels wherein the Cₘₐₓ to C_{average} ratio does not exceed five or does not exceed three for at least one month following a single administration; (3) release within effective levels for at least 56 days following a single administration; and/or (4) release within effective levels wherein the Cₘₐₓ to C_{average} ratio does not exceed five or does not exceed three for at least 56 days following a single administration.
